SEQUENCE LISTING (65 sequences)

SEQ ID NO: 1 - protein, 1971 aa, Artificial Sequence
  sCR1
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHD

SEQ ID NO: 2 - protein, 1930 aa, Artificial Sequence
  sCR1(1971)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHD

SEQ ID NO: 3 - protein, 1351 aa, Artificial Sequence
  sCR1(1392)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVR

SEQ ID NO: 4 - protein, 898 aa, Artificial Sequence
  sCR1(939)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGI

SEQ ID NO: 5 - protein, 903 aa, Artificial Sequence
  sCR1(490-1392)
LGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVR

SEQ ID NO: 6 - protein, 1482 aa, Artificial Sequence
  sCR1(490-1971)
LGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHD

SEQ ID NO: 7 - protein, 193 aa, Artificial Sequence
  sCR1(234)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCII

SEQ ID NO: 8 - protein, 448 aa, Artificial Sequence
  sCR1(489)
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGI

SEQ ID NO: 9 - protein, 1482 aa, Artificial Sequence; identical to SEQ ID NO: 6
  sCR1(940-1971)

SEQ ID NO: 10 - protein, 450 aa, Artificial Sequence
  sCR1(490-939)
LGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGI

SEQ ID NO: 11 - protein, 903 aa, Artificial Sequence; identical to SEQ ID NO: 5
  sCR1(940-1392)

SEQ ID NO: 12 - protein, 579 aa, Artificial Sequence
  sCR1(1393-1971)
AGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHD

SEQ ID NO: 13 - protein, 448 aa, Artificial Sequence; identical to SEQ ID NO: 8
  sCR1 LHR-A

SEQ ID NO: 14 - protein, 450 aa, Artificial Sequence; identical to SEQ ID NO: 10
  sCR1 LHR-B

SEQ ID NO: 15 - protein, 453 aa, Artificial Sequence
  sCR1 LHR-C
LGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVR

SEQ ID NO: 16 - protein, 579 aa, Artificial Sequence; identical to SEQ ID NO: 12
  sCR1 LHR-D

SEQ ID NO: 17 - protein, 8 aa, Artificial Sequence
  8xHis Tag
HHHHHHHH

SEQ ID NO: 18 - protein, 41 aa, Artificial Sequence
  endogenous signal peptide
MGASSPRSPEPVGPPAPGLPFCCGGSLLAVVVLLALPVAWG

SEQ ID NO: 19 - protein, 19 aa, Artificial Sequence
  exogenous signal peptide
MKILILGIFLFLCSTPAWA

SEQ ID NO: 20 - protein, 1979 aa, Artificial Sequence
  sCR1(1971)-8His
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDHHHHHHHH

SEQ ID NO: 21 - protein, 1400 aa, Artificial Sequence
  sCR1(1392)-8His
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRHHHHHHHH

SEQ ID NO: 22 - protein, 947 aa, Artificial Sequence
  sCR1(939)-8His
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGIHHHHHHHH

SEQ ID NO: 23 - protein, 930 aa, Artificial Sequence
  sCR1(490-1392)-8His
MKILILGIFLFLCSTPAWALGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRHHHHHHHH

SEQ ID NO: 24 - protein, 1509 aa, Artificial Sequence
  sCR1(490-1971)-8His
MKILILGIFLFLCSTPAWALGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDHHHHHHHH

SEQ ID NO: 25 - protein, 242 aa, Artificial Sequence
  sCR1(234)-8His
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIHHHHHHHH

SEQ ID NO: 26 - protein, 497 aa, Artificial Sequence
  sCR1(489)-8His
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGIHHHHHHHH

SEQ ID NO: 27 - protein, 1059 aa, Artificial Sequence
  sCR1(940-1971)-8His
MKILILGIFLFLCSTPAWALGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDHHHHHHHH

SEQ ID NO: 28 - protein, 477 aa, Artificial Sequence
  sCR1(490-939)-8His
MKILILGIFLFLCSTPAWALGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGIHHHHHHHH

SEQ ID NO: 29 - protein, 480 aa, Artificial Sequence
  sCR1(940-1392)-8His
MKILILGIFLFLCSTPAWALGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRHHHHHHHH

SEQ ID NO: 30 - protein, 606 aa, Artificial Sequence
  sCR1(1393-1971)-8His
MKILILGIFLFLCSTPAWAAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDHHHHHHHH

SEQ ID NO: 31 - protein, 13 aa, Artificial Sequence
  Linker
GSGGSGGSGGSGS

SEQ ID NO: 32 - protein, 15 aa, Artificial Sequence
  GS15 peptide linker
GGGGSGGGGSGGGGS

SEQ ID NO: 33 - protein, 16 aa, Artificial Sequence
  GS16 peptide linker
SGGGGSGGGGSGGGGS

SEQ ID NO: 34 - protein, 20 aa, Artificial Sequence
  GS20 peptide linker
GGGGSGGGGSGGGGSGGGGS

SEQ ID NO: 35 - protein, 30 aa, Artificial Sequence
  GS30 peptide linker
SGGSGGSGGSGGSGGSGGSGGSGGSGGSGS

SEQ ID NO: 36 - protein, 118 aa, Artificial Sequence
  amino acid sequence of C1.2 VH
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSS

SEQ ID NO: 37 - protein, 107 aa, Artificial Sequence
  amino acid sequence of C1.2 VL
DIQMTQSPSALSASVGDRVTITCRASQGISSYLNWYQQKPGKAPKLLIYYASNLQNGIPSRFSGSGSGTDFTLTISSLQPEDFATYHCQQSYSTPLTFGGGTNVEIR

SEQ ID NO: 38 - protein, 118 aa, Artificial Sequence; identical to SEQ ID NO: 36
  amino acid sequence of C1.2G VH

SEQ ID NO: 39 - protein, 107 aa, Artificial Sequence
  amino acid sequence of C1.2G VL
DIQMTQSPSSLSASVGDRVTITCRASQGISSYLNWYQQKPGKAPKLLIYYASNLQNGVPSRFSGSGSGTDFTLTISSLQPEDFATYYCQQSYSTPLTFGGGTKVEIK

SEQ ID NO: 40 - protein, 5 aa, Artificial Sequence
  amino acid sequence of C1.2 VH CDR1
LYWMG

SEQ ID NO: 41 - protein, 17 aa, Artificial Sequence
  amino acid sequence of C1.2 VH CDR2
SISSSGGVTPYADSVKG

SEQ ID NO: 42 - protein, 9 aa, Artificial Sequence
  amino acid sequence of C1.2 VH CDR3
LGELGWFDP

SEQ ID NO: 43 - protein, 11 aa, Artificial Sequence
  amino acid sequence of C1.2 VL CDR1
RASQGISSYLN

SEQ ID NO: 44 - protein, 6 aa, Artificial Sequence
  amino acid sequence of C1.2 VL CDR2
ASNLQN

SEQ ID NO: 45 - protein, 9 aa, Artificial Sequence
  amino acid sequence of C1.2 VL CDR3
QQSYSTPLT

SEQ ID NO: 46 - protein, 120 aa, Artificial Sequence
  amino acid sequence of 5E2VR81 VH
EVQLLESGGGLVQPGGSLRLSCAASGFTFSYYAMWWVRQAPGKGLEWVSLIDVSGGVQLYADSVKGRFTISRDNSKNTLYLQMNSLRAEDTAVYYCAKPLIGTRDAFDIWGQGTMVTVSS

SEQ ID NO: 47 - protein, 108 aa, Artificial Sequence
  amino acid sequence of 5E2VR81 VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIKR

SEQ ID NO: 48 - protein, 319 aa, Artificial Sequence
  amino acid sequence 25-335 of Homo sapiens G-CSFR (hG-CSFR) with a C-terminal polyhistidine tag
ECGHISVSAPIVHLGDPITASCIIKQNCSHLDPEPQILWRLGAELQPGGRQQRLSDGTQESIITLPHLNHTQAFLSCALNWGNSLQILDQVELRAGYPPAIPHNLSCLMNLTTSSLICQWEPGPETHLPTSFTLKSFKSRGNCQTQGDSILDCVPKDGQSHCSIPRKHLLLYQNMGIWVQAENALGTSMSPQLCLDPMDVVKLEPPMLRTMDPSPEAAPPQAGCLQLSWEPWQPGLHINQKCELRHKPQRGEASWALVGPLPLEALQYELCGLLPATAYTLQIRCIRWPLPGHWSDWSPSLELRTTERAPTHHHHHHHH

SEQ ID NO: 49 - protein, 1649 aa, Artificial Sequence
  amino acid sequence of truncated soluble complement receptor 1 conjugated to C1.2scFv (sCR1(1392)-GS16-C1.2scFvLH) with N-terminal endogenous signal peptide
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRSGGGGSGGGGSGGGGSDIQMTQSPSSLSASVGDRVTITCRASQGISSYLNWYQQKPGKAPKLLIYYASNLQNGVPSRFSGSGSGTDFTLTISSLQPEDFATYYCQQSYSTPLTFGGGTKVEIKRGGGGSGGGGSGGGGSEVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSS

SEQ ID NO: 50 - protein, 1649 aa, Artificial Sequence
  amino acid sequence of truncated soluble complement receptor 1 conjugated to C1.2scFv (sCR1(1392)-GS16-C1.2scFvHL) with N-terminal endogenous signal peptide
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRSGGGGSGGGGSGGGGSEVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSSGGGGSGGGGSGGGGSDIQMTQSPSSLSASVGDRVTITCRASQGISSYLNWYQQKPGKAPKLLIYYASNLQNGVPSRFSGSGSGTDFTLTISSLQPEDFATYYCQQSYSTPLTFGGGTKVEIKR

SEQ ID NO: 51 - protein, 1656 aa, Artificial Sequence
  amino acid sequence of truncated soluble complement receptor 1 conjugated to 5E2VR81scFv (sCR1(1392)-GS16-5E2VR81scFvLH) with N-terminal endogenous signal peptide
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIKRGGGGSGGGGSGGGGSGGGGSEVQLLESGGGLVQPGGSLRLSCAASGFTFSYYAMWWVRQAPGKGLEWVSLIDVSGGVQLYADSVKGRFTISRDNSKNTLYLQMNSLRAEDTAVYYCAKPLIGTRDAFDIWGQGTMVTVSS

SEQ ID NO: 52 - protein, 1656 aa, Artificial Sequence
  amino acid sequence of truncated soluble complement receptor 1 conjugated to 5E2VR81scFv (sCR1(1392)- GS16-5E2VR81scFvHL) with N-terminal endogenous signal peptide
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRSGGGGSGGGGSGGGGSEVQLLESGGGLVQPGGSLRLSCAASGFTFSYYAMWWVRQAPGKGLEWVSLIDVSGGVQLYADSVKGRFTISRDNSKNTLYLQMNSLRAEDTAVYYCAKPLIGTRDAFDIWGQGTMVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIKR

SEQ ID NO: 53 - protein, 1648 aa, Artificial Sequence
  amino acid sequence of truncated soluble complement receptor 1 conjugated to C1.2GscFv (sCR1(1392)- GS16-C1.2GscFvLH) with N-terminal endogenous signal peptide
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRSGGGGSGGGGSGGGGSDIQMTQSPSSLSASVGDRVTITCRASQGISSYLNWYQQKPGKAPKLLIYYASNLQNGVPSRFSGSGSGTDFTLTISSLQPEDFATYYCQQSYSTPLTFGGGTKVEIKGGGGSGGGGSGGGGSEVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSS

SEQ ID NO: 54 - protein, 1648 aa, Artificial Sequence
  amino acid sequence of truncated soluble complement receptor 1 conjugated to C1.2GscFv (sCR1(1392)- GS16-C1.2GscFvHL) with N-terminal endogenous signal peptide
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRSGGGGSGGGGSGGGGSEVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSSGGGGSGGGGSGGGGSDIQMTQSPSSLSASVGDRVTITCRASQGISSYLNWYQQKPGKAPKLLIYYASNLQNGVPSRFSGSGSGTDFTLTISSLQPEDFATYYCQQSYSTPLTFGGGTKVEIK

SEQ ID NO: 55 - protein, 16 aa, Artificial Sequence
  GS16 peptide linker
GGGGSGGGGSGGGGGS

SEQ ID NO: 56 - protein, 129 aa, Artificial Sequence
  amino acid sequence of 3F7 VH
EVQLLESGGGLVQPGGSLRLSCAASGFTFSKYIMQWVRQAPGKGLEWVSGIRPSGGTTVYADSVKGRFTISRDNSKNTLYLQMNSLRAEDTAVYYCARALPRSGYLISPHYYYYALDVWGQGTTVTVSS

SEQ ID NO: 57 - protein, 110 aa, Artificial Sequence
  amino acid sequence of 3F7 VL
QSELTQPPSASGTPGQRVTISCSGSSSNIGRNYVYWYQQVPGTAPKLLIYSNNQRPSGVPDRFSGSKSGTSASLVISGLRSEDEADYYCAAWDASLRGVFGGGTKLTVLG

SEQ ID NO: 58 - protein, 129 aa, Artificial Sequence; identical to SEQ ID NO: 56
  amino acid sequence of 3F7G VH

SEQ ID NO: 59 - protein, 110 aa, Artificial Sequence
  amino acid sequence of 3F7G VL
QSVLTQPPSASGTPGQRVTISCSGSSSNIGRNYVYWYQQLPGTAPKLLIYSNNQRPSGVPDRFSGSKSGTSASLAISGLRSEDEADYYCAAWDASLRGVFGGGTKLTVLG

SEQ ID NO: 60 - protein, 150 aa, Artificial Sequence
  amino acid sequence of affinity matured 3F7 VH
EVQLLESGGGLVQPGGSLRLSCAASGFTFSKYIMQWVRQAPGKGLEWVSGIDIPTKGTVYADSVKGRFTISRDNSKNTLYLQMNSLRAEDTAVYYCARALPRSGYLISPHYYYYALDVWGQGTTVTVSSASTKGPSVFPLAPCSRSTSES

SEQ ID NO: 61 - protein, 110 aa, Artificial Sequence; identical to SEQ ID NO: 59
  amino acid sequence of affinity matured 3F7 VL

SEQ ID NO: 62 - protein, 1663 aa, Artificial Sequence
  amino acid sequence of truncated soluble complement receptor 1 conjugated to 3F7scFv (sCR1(1392)-GS16- 3F7scFvHL) with N-terminal endogenous signal peptide
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRSGGGGSGGGGSGGGGSEVQLLESGGGLVQPGGSLRLSCAASGFTFSKYIMQWVRQAPGKGLEWVSGIRPSGGTTVYADSVKGRFTISRDNSKNTLYLQMNSLRAEDTAVYYCARALPRSGYLISPHYYYYALDVWGQGTTVTVSSGGGGSGGGGSGGGGGSQSELTQPPSASGTPGQRVTISCSGSSSNIGRNYVYWYQQVPGTAPKLLIYSNNQRPSGVPDRFSGSKSGTSASLVISGLRSEDEADYYCAAWDASLRGVFGGGTKLTVLG

SEQ ID NO: 63 - protein, 1663 aa, Artificial Sequence
  amino acid sequence of truncated soluble complement receptor 1 conjugated to 3F7GscFv (sCR1(1392)- GS16-3F7GscFvHL) with N-terminal endogenous signal peptide
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRSGGGGSGGGGSGGGGSEVQLLESGGGLVQPGGSLRLSCAASGFTFSKYIMQWVRQAPGKGLEWVSGIRPSGGTTVYADSVKGRFTISRDNSKNTLYLQMNSLRAEDTAVYYCARALPRSGYLISPHYYYYALDVWGQGTTVTVSSGGGGSGGGGSGGGGGSQSVLTQPPSASGTPGQRVTISCSGSSSNIGRNYVYWYQQLPGTAPKLLIYSNNQRPSGVPDRFSGSKSGTSASLAISGLRSEDEADYYCAAWDASLRGVFGGGTKLTVLG

SEQ ID NO: 64 - protein, 1663 aa, Artificial Sequence
  amino acid sequence of truncated soluble complement receptor 1 conjugated to 3F7affscFv (sCR1(1392)- GS16-3F7affscFvHL) with N-terminal endogenous signal peptide
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRSGGGGSGGGGSGGGGSEVQLLESGGGLVQPGGSLRLSCAASGFTFSKYIMQWVRQAPGKGLEWVSGIDIPTKGTVYADSVKGRFTISRDNSKNTLYLQMNSLRAEDTAVYYCARALPRSGYLISPHYYYYALDVWGQGTTVTVSSGGGGSGGGGSGGGGGSQSVLTQPPSASGTPGQRVTISCSGSSSNIGRNYVYWYQQLPGTAPKLLIYSNNQRPSGVPDRFSGSKSGTSASLAISGLRSEDEADYYCAAWDASLRGVFGGGTKLTVLG

SEQ ID NO: 65 - protein, 615 aa, Homo sapiens
MRALLLLGFLLVSLESTLSIPPWEAPKEHKYKAEEHTVVLTVTGEPCHFPFQYHRQLYHKCTHKGRPGPQPWCATTPNFDQDQRWGYCLEPKKVKDHCSKHSPCQKGGTCVNMPSGPHCLCPQHLTGNHCQKEKCFEPQLLRFFHKNEIWYRTEQAAVARCQCKGPDAHCQRLASQACRTNPCLHGGRCLEVEGHRLCHCPVGYTGAFCDVDTKASCYDGRGLSYRGLARTTLSGAPCQPWASEATYRNVTAEQARNWGLGGHAFCRNPDNDIRPWCFVLNRDRLSWEYCDLAQCQTPTQAAPPTPVSPRLHVPLMPAQPAPPKPQPTTRTPPQSQTPGALPAKREQPPSLTRNGPLSCGQRLRKSLSSMTRVVGGLVALRGAHPYIAALYWGHSFCAGSLIAPCWVLTAAHCLQDRPAPEDLTVVLGQERRNHSCEPCQTLAVRSYRLHEAFSPVSYQHDLALLRLQEDADGSCALLSPYVQPVCLPSGAARPSETTLCQVAGWGHQFEGAEEYASFLQEAQVPFLSLERCSAPDVHGSSILPGMLCAGFLEGGTDACQGDSGGPLVCEDQAAERRLTLQGIISWGSGCGDRNKPGVYTDVAYYLAWIREHTVS

(19) United States
(12) Patent Application Publication
(10) Pub. No.: US 2022/0305133 A1
(43) Pub. Date: Sep. 29, 2022

(54) Soluble Complement Receptor Type 1 Variant Conjugates and Uses Thereof

(71) Applicant: CSL Innovation Pty., Ltd., Parkville, Victoria (AU)

(72) Inventor: Matthew Hardy, Doreen, Victoria (AU)

(73) Assignee: CSL Innovation Pty., Ltd., Parkville, Victoria (AU)

(21) Appl. No.: 17/618,076

(22) PCT Filed: Jun. 12, 2020
(86) PCT No.: PCT/AU2020/050600

§ 371 Date: Dec. 10, 2021

(30) Foreign Application Priority Data
Jun. 12, 2019 (AU) 2019902044
Jun. 12, 2019 (AU) 2019902053

(51) Int. Cl. A61K 47/68; A61P 7/02

(52) U.S. Cl. CPC A61K 47/6849; A61K 47/6811; A61K 47/6845; A61P 7/02

(57) ABSTRACT

A soluble complement receptor type 1 (sCR1) conjugate comprising a sCR1 variant and a) a protein comprising an antigen binding domain that binds to a target and inhibits signaling by or via the target; orb) a protein comprising an antigen binding domain that binds to a blood coagulation factor.

### RELATED APPLICATION DATA

The present application claims priority from Australian Patent Application No. 2019902044 entitled “Soluble complement receptor type I variant conjugates and uses thereof” filed on 12 Jun. 2019 and from Australian Patent Application No. 2019902053 entitled “Soluble complement receptor type I variant conjugates and uses thereof” filed on 12 Jun. 2019. The entire contents of both is hereby incorporated by reference.

### SEQUENCE LISTING

The present application is filed with a Sequence Listing in electronic form. The entire contents of the Sequence Listing are hereby incorporated by reference.

### FIELD

The present disclosure relates to soluble complement receptor type 1 variant conjugates and uses thereof.

### BACKGROUND

The innate immune system is one of the body's first non-specific defence systems in response to a foreign antigen and functions to recruit immune cells to sites of infection through the production of chemical factors, activation of the complement cascade and the adaptive immune system, as well as acting as a physical and chemical barrier to infectious agents.

As part of the innate immune system, the complement system is comprised of a number of cell-surface and soluble proteins that play a role in elimination of foreign microorganisms, whilst protecting the host from complement-related damage. Activation of the complement system leads to increased vascular permeability, chemotaxis of phagocytic cells, activation of inflammatory cells, opsonization of foreign particles, direct killing of cells and tissue damage.

Neutrophils are the most abundant type of granulocytes and the most abundant (60% to 70%) type of white blood cells in mammals and are important components of the innate immune system. Neutrophils are one of the first cell types of the innate immune system to travel to the site of an infection and help fight infection by ingesting microorganisms and releasing enzymes that kill the microorganisms.

Granulocyte-colony stimulating factor (G-CSF) promotes expansion and maturation of neutrophil populations. G-CSF is a major regulator of granulocyte production. G-CSF is produced by bone marrow stromal cells, endothelial cells, macrophages, and fibroblasts and production is induced by inflammatory stimuli. G-CSF acts through the G-CSF receptor (G-CSFR), which is expressed on early myeloid progenitors, mature neutrophils, monocytes/macrophages, T and B lymphocytes and endothelial cells. Mice deficient in G-CSF or the G-CSFR exhibit marked neutropenia, demonstrating the importance of G-CSF in steady-state granulopoiesis. G-CSF increases the production and release of neutrophils, mobilizes hematopoietic stem cells and progenitor cells, and modulates the differentiation, lifespan, and effector functions of mature neutrophils. G-CSF may also exert effects on macrophages, including expansion of monocyte/macrophage numbers, enhancement of phagocytic function, and regulation of inflammatory cytokine and chemokine production. G-CSF has also been shown to mobilize endothelial progenitor cells and induce or promote angiogenesis.

While the innate immune system is important in protecting a subject, dysregulation of this system can lead to disease. Working with the innate immune system during this dysregulation is the blood coagulation system. Activation of the coagulation cascade limits pathogen dissemination and supports pathogen killing

Normal blood coagulation is a highly conserved process in mammalian biology 20 involving complex physiological and biochemical processes comprising activation of a coagulation factor (or clotting factor) cascade ultimately leading to fibrin formation and platelet aggregation. The blood coagulation cascade comprises an “extrinsic” pathway, the primary means of coagulation initiation, and an “intrinsic” pathway, which contributes to stabilisation of the fibrin clot.

The majority of coagulation factors involved in the coagulation cascade are precursors of proteolytic enzymes known as zymogens. These enzymes circulate in the blood in a non-activated form and only participate in the coagulation cascade once they become activated (e.g. by proteolytic cleavage).

Factor XII (FXII, Hageman factor) is an essential coagulation protein for initiation of the intrinsic coagulation cascade. Activation of FXII to produce activated FXII (FXIIa) leads to activation of Factor XI to Factor XIa and C1 esterases (C1r, C1s), the first components of the macromolecular complex of C1 and the classic complement cascade. Activation of FXI leads to a series of proteolytic reactions resulting in thrombin generation and the hemostatic pathway, whilst activation of the complement system leads to increased vascular permeability, chemotaxis of phagocytic cells, activation of inflammatory cells, opsonization of foreign particles, direct killing of cells and tissue damage.

Despite its role in activation of the intrinsic coagulation cascade, and activation of the classical complement system, deficiencies in Factor XII are not associated with bleeding abnormalities. However, dysregulation of these pathways can lead to serious conditions, with both FXII- and complement-deficiencies being shown to be associated with pathological thrombus formation and stroke.

The innate immune system is clearly important in the immune system in disease, as is the regulation of Factor XII and the intrinsic coagulation pathway in maintaining coagulation homeostasis. However, both these systems are controlled by numerous proteins, many of which have distinct and non-redundant actions. For example, complement receptor type 1 (CR1) is a principal regulator of the activation of complement. CR1 (also known as C3b/C4b receptor) is a membrane-bound protein present on erythrocytes, macrophages/monocytes, granulocytes, B cells, some T cells, splenic follicular dendritic cells and glomerular podocytes. A minor amount of soluble CR1 (sCR1) is cleaved from the cell surface CR1. A recombinant version of this soluble molecule has previously been generated and is known as TP10. CR1 is a negative regulator of C3 activation and thus sCR1 can inhibit each of the classical, lectin and alternative pathways. Dysregulation of the complement system has been shown to be associated with ischemia-reperfusion injury, asthma, allergy, cancer, and autoimmune disease such as systemic lupus erythematosus, Sjogren's Syndrome (SS), antiphospholipid syndrome (APS), rheumatoid arthritis (RA), vasculitis, multiple sclerosis and dermatomyositis.

Accordingly, there remains a need to develop therapeutics that are capable of targeting multiple pathways in disease, such as complement-mediated disorders, neutrophil-mediated disorders and/or blood coagulation disorders. It will be clear to the skilled person that there is a need in the art for such therapeutics with improved activity, such as increased complement inhibitory activity, and/or increased half-life.

### SUMMARY

In producing the present disclosure, the inventors produced soluble complement receptor type 1 (sCR1) truncation variants, e.g., variants comprising defined amino acid sequences corresponding to one or more long homologous repeat (LHR) regions (i.e., LHR-A, LHR-B, LHR-C and/or LHR-D) conjugated to proteins comprising an antigen 35 binding domain (e.g., a protein that inhibits G-CSF signalling or a protein that antagonises activation and/or activity of Factor XII/Factor XIIa). The inventors found that for the sCR1 conjugates, expression products were soluble, expressed at a high level and at the expected size. The inventors studied the effects of the sCR1 conjugates for complement inhibiting activity. Importantly, the inventors found that conjugation of the sCR1 variants to proteins comprising an antigen binding domain (e.g., a protein that inhibits G-CSF signalling or a protein that antagonises activation and/or activity of Factor XII/Factor XIIa) had no deleterious effect on the potency for complement pathway inhibition.

The findings by the inventors provide the basis for soluble complement receptor type 1 (sCR1) conjugates comprising a sCR1 variant and a protein comprising an antigen binding domain that binds to a target and inhibits signaling by or via the target.

The findings by the inventors also provide the basis for soluble complement receptor type 1 (sCR1) conjugates comprising a sCR1 variant and a protein comprising an antigen binding domain that binds to a blood coagulation factor.

The findings by the inventors also provide the basis for methods of inhibiting complement activity in a subject, comprising administering a sCR1 conjugate to the subject. Furthermore, the findings by the inventors provide the basis for methods for treating or preventing a disorder, e.g., a complement-mediated disorder, a neutrophil-mediated disorder and/or a blood coagulation disorder, in a subject.

The present disclosure provides, a soluble complement receptor type 1 (sCR1) conjugate comprising:
(i) a sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising an antigen binding domain that binds to a target and inhibits or antagonizes the target.

The present disclosure provides, a soluble complement receptor type 1 (sCR1) conjugate comprising:
(i) a sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising an antigen binding domain that binds to a target and inhibits signaling by or via the target.

The present disclosure also provides, a soluble complement receptor type 1 (sCR1) conjugate comprising:
(i) a sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising an antigen binding domain that binds to a blood coagulation factor.

The present disclosure also provides, a soluble complement receptor type 1 (sCR1) conjugate comprising:
(i) a sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising an antigen binding domain that binds to a zymogen of a blood clotting factor and antagonizes activation of the blood coagulation factor.

In one example, the sCR1 variant comprises:
(i) an amino acid sequence corresponding to amino acids 42 to 1392 of SEQ ID NO: 1 (e.g., lacking amino acid residues 1393 to 1971 of SEQ ID NO: 1);
(ii) an amino acid sequence corresponding to amino acids 42 to 939 of SEQ ID NO: 1 (e.g., lacking amino acid residues 940 to 1971 of SEQ ID NO: 1);
(iii) an amino acid sequence corresponding to amino acids 490 to 1392 of SEQ ID NO: 1 (e.g., lacking amino acid residues 1 to 489 and 1393 to 1971 of SEQ ID NO: 1); or
(iv) an amino acid sequence corresponding to amino acids 490 to 1971 of SEQ ID NO: 1 (e.g., lacking amino acid residues 1 to 489 of SEQ ID NO: 1).

In one example, the sCR1 variant comprises an amino acid sequence corresponding to amino acids 42 to 1392 of SEQ ID NO: 1 (e.g., lacking amino acid residues 1393 to 1971 of SEQ ID NO: 1).

In one example, the sCR1 variant comprises an amino acid sequence corresponding to amino acids 42 to 939 of SEQ ID NO: 1 (e.g., lacking amino acid residues 940 to 1971 of SEQ ID NO: 1).

In one example, the sCR1 variant comprises an amino acid sequence corresponding to amino acids 490 to 1392 of SEQ ID NO: 1 (e.g., lacking amino acid residues 1 to 489 and 1393 to 1971 of SEQ ID NO: 1).

In one example, the sCR1 variant comprises an amino acid sequence corresponding to amino acids 490 to 1971 of SEQ ID NO: 1 (e.g., lacking amino acid residues 1 to 489 of SEQ ID NO: 1).

In one example, the sCR1 variant consists of:
(i) an amino acid sequence corresponding to amino acids 42 to 1392 of SEQ ID NO: 1;
(ii) an amino acid sequence corresponding to amino acids 42 to 939 of SEQ ID NO: 1;
(iii) an amino acid sequence corresponding to amino acids 490 to 1392 of SEQ ID NO: 1; or
(iv) an amino acid sequence corresponding to amino acids 490 to 1971 of SEQ ID NO: 1.

In one example, the sCR1 variant consists of an amino acid sequence corresponding to amino acids 42 to 1392 of SEQ ID NO: 1 or comprises an amino acid sequence corresponding to amino acids 42 to 1392 of SEQ ID NO: 1 (e.g., lacking amino acid residues 1393 to 1971 of SEQ ID NO: 1). The inventors have shown that such a sCR1 variant has improved complement inhibitory activity compared to a sCR1 variant comprising amino acids 42 to 1971 of SEQ ID NO: 1. This finding was unexpected since the region of CR1 in amino acids 1393 to 1971 binds to C1q and mannose binding lectin (MBL), and its removal might reasonably have been expected to be deleterious to complement inhibitory activity or to have no effect.

In one example, the sCR1 variant consists of an amino acid sequence corresponding to amino acids 42 to 939 of SEQ ID NO: 1.

In one example, the sCR1 variant consists of an amino acid sequence corresponding to amino acids 490 to 1392 of SEQ ID NO: 1.

In one example, the sCR1 variant consists of an amino acid sequence corresponding to amino acids 490 to 1971 of SEQ ID NO: 1.

In one example, the sCR1 variant does not consist or comprise an amino acid sequence corresponding to amino acids 1 to 1971 of SEQ ID NO: 1.

In one example, the sCR1 variant does not consist or comprise an amino acid sequence corresponding to amino acids 42 to 1971 of SEQ ID NO: 1.

In one example, the sCR1 variant of the present disclosure optionally comprises one or more amino acid substitutions, deletions or insertions of any sequence disclosed herein. Amino acid substitutions suitable for use in the present disclosure will be apparent to the skilled person and include naturally-occurring substitutions and engineered substitutions.

In one example, a sCR1 variant of the present disclosure comprises one or more conservative amino acid substitutions compared to a sequence disclosed herein. In some examples, the sCR1 variant comprises 10 or fewer, e.g., 9 or 8 or 7 or 6 or 5 or 4 or 3 or 2 or 1 conservative amino acid substitutions.

In one example, a sCR1 variant of the present disclosure comprises one or more non-conservative amino acid changes. For example, non-conservative amino acid substitutions increase half-life, reduce immunogenicity, and/or increase inhibitory activity of a sCR1 variant of the present disclosure. In one example, the sCR1 variant comprises fewer than 6 or 5 or 4 or 3 or 2 or 1 non-conservative amino acid substitutions.

In one example, a sCR1 variant of the present disclosure comprises a sequence at least about 85% or about 90% or about 95% or about 97% or about 98% or about 99% identical to a sequence disclosed herein.

In one example, the sCR1 variant comprises an amino acid sequence at least about 85% or about 90% or about 95% or about 97% or about 98% or about 99% identical to an amino acid sequence corresponding to amino acids 42 to 1392 of SEQ ID NO: 1 (e.g., lacking amino acid residues 1393 to 1971 of SEQ ID NO: 1). For example, the sCR1 variant comprises an amino acid sequence about 85% identical to an amino acid sequence corresponding to amino acids 42 to 1392 of SEQ ID NO: 1. In another example, the sCR1 variant comprises an amino acid sequence about 90% identical to an amino acid sequence corresponding to amino acids 42 to 1392 of SEQ ID NO: 1. In another example, the sCR1 variant comprises an amino acid sequence about 95% identical to an amino acid sequence corresponding to amino acids 42 to 1392 of SEQ ID NO: 1. In a further example, the sCR1 variant comprises an amino acid sequence about 97% identical to an amino acid sequence corresponding to amino acids 42 to 1392 of SEQ ID NO: 1. In one example, the sCR1 variant comprises an amino acid sequence about 98% identical to an amino acid sequence corresponding to amino acids 42 to 1392 of SEQ ID NO: 1. In another example, the sCR1 variant comprises an amino acid sequence about 99% identical to an amino acid sequence corresponding to amino acids 42 to 1392 of SEQ ID NO: 1.

In one example, the sCR1 variant consists of an amino acid sequence at least about 85% or about 90% or about 95% or about 97% or about 98% or about 99% identical to an amino acid sequence corresponding to amino acids 42 to 939 of SEQ ID NO: 1. For example, the sCR1 variant comprises an amino acid sequence about 85% identical to an amino acid sequence corresponding to amino acids 42 to 939 of SEQ ID NO: 1. In another example, the sCR1 variant comprises an amino acid sequence about 90% identical to an amino acid sequence corresponding to amino acids 42 to 939 of SEQ ID NO: 1. In another example, the sCR1 variant comprises an amino acid sequence about 95% identical to an amino acid sequence corresponding to amino acids 42 to 939 of SEQ ID NO: 1. In a further example, the sCR1 variant comprises an amino acid sequence about 97% identical to an amino acid sequence corresponding to amino acids 42 to 939 of SEQ ID NO: 1. In one example, the sCR1 variant comprises an amino acid sequence about 98% identical to an amino acid sequence corresponding to amino acids 42 to 939 of SEQ ID NO: 1. In another example, the sCR1 variant comprises an amino acid sequence about 99% identical to an amino acid sequence corresponding to amino acids 42 to 939 of SEQ ID NO: 1.

In one example, the sCR1 variant consists of an amino acid sequence at least about 85% or about 90% or about 95% or about 97% or about 98% or about 99% identical to an amino acid sequence corresponding to amino acids 490 to 1392 of SEQ ID NO: 1. For example, the sCR1 variant comprises an amino acid sequence about 85% identical to an amino acid sequence corresponding to amino acids 490 to 1392 of SEQ ID NO: 1. In another example, the sCR1 variant comprises an amino acid sequence about 90% identical to an amino acid sequence corresponding to amino acids 490 to 1392 of SEQ ID NO: 1. In another example, the sCR1 variant comprises an amino acid sequence about 95% identical to an amino acid sequence corresponding to amino acids 490 to 1392 of SEQ ID NO: 1. In a further example, the sCR1 variant comprises an amino acid sequence about 97% identical to an amino acid sequence corresponding to amino acids 490 to 1392 of SEQ ID NO: 1. In one example, the sCR1 variant comprises an amino acid sequence about 98% identical to an amino acid sequence corresponding to amino acids 490 to 1392 of SEQ ID NO: 1. In another example, the sCR1 variant comprises an amino acid sequence about 99% identical to an amino acid sequence corresponding to amino acids 490 to 1392 of SEQ ID NO: 1.

In one example, the sCR1 variant consists of an amino acid sequence at least about 85% or about 90% or about 95% or about 97% or about 98% or about 99% identical to an amino acid sequence corresponding to amino acids 490 to 1971 of SEQ ID NO: 1. For example, the sCR1 variant comprises an amino acid sequence about 85% identical to an amino acid sequence corresponding to amino acids 490 to 1971 of SEQ ID NO: 1. In another example, the sCR1 variant comprises an amino acid sequence about 90% identical to an amino acid sequence corresponding to amino acids 490 to 1971 of SEQ ID NO: 1. In another example, the sCR1 variant comprises an amino acid sequence about 95% identical to an amino acid sequence corresponding to amino acids 490 to 1971 of SEQ ID NO: 1. In a further example, the sCR1 variant comprises an amino acid sequence about 97% identical to an amino acid sequence corresponding to amino acids 490 to 1971 of SEQ ID NO: 1. In one example, the sCR1 variant comprises an amino acid sequence about 98% identical to an amino acid sequence corresponding to amino acids 490 to 1971 of SEQ ID NO: 1. In another example, the sCR1 variant comprises an amino acid sequence about 99% identical to an amino acid sequence corresponding to amino acids 490 to 1971 of SEQ ID NO: 1.

In one example, the sCR1 variant of the present disclosure has increased complement inhibitory activity compared to a sCR1 comprising a sequence set forth in SEQ ID NO: 2. For example, the complement inhibitory activity of the sCR1 variant of the present disclosure is increased by at least about 1.5 fold, or about 2 fold, or about 3 fold, or about 3.5 fold, or about 4 fold, or about 5 fold, or about 6 fold, or about 8 fold, or about 10 fold compared to a sCR1 comprising a sequence set forth in SEQ ID NO: 2.

Methods for determining the inhibitory activity of the sCR1 variant and/or sCR1 conjugate will be apparent to the skilled person and/or described herein. In one example, complement inhibitory activity is determined using an in vitro assay. For example, complement activity is measured using an enzyme immunoassay (e.g., an immunoassay that measures complement activation, such as a Wieslab® complement assay kit). For example, complement inhibitory activity is determined using labelled antibodies specific for an antigen or an epitope produced during complement activation (e.g., C5b-9 or an epitope present in C5b-C9). In one example, the wells of a microtitre plate are coated with specific activators of the classical, lectin or alternative pathway. In one example, the sCR1 variant and/or conjugate is incubated with normal human serum and appropriate assay diluent (i.e., a diluent comprising appropriate blocking components to ensure specific activation of the classical, lectin or alternative pathway) and added to microtitre plate wells coated with specific activators of the classical, lectin or alternative pathway and the amount of C5b-9 complex formed is detected using a specific alkaline phosphatase labelled antibody to the C5b-9. In one example, the amount of complement activation product (i.e., C5b-9) produced is proportional to the functional activity of the complement pathway. In one example, the half maximal inhibitor concentration (i.e., IC₅₀) is determined. For example, the IC₅₀ of the sCR1 variant is determined and compared to the IC₅₀ of a sCR1 comprising a sequence set forth in SEQ ID NO: 2. In another example, complement inhibitory activity is determined using a hemolysis assay (e.g., classical pathway (i.e., CH50) and alternative pathway (ApH50) inhibition assays).

In one example, the sCR1 variant has increased complement inhibitory activity in the classical pathway, the lectin pathway and/or alternative complement pathway compared to a sCR1 comprising a sequence set forth in SEQ ID NO: 2.

In one example, the sCR1 variant has increased inhibitory activity in the classical complement pathway compared to a sCR1 comprising a sequence set forth in SEQ ID NO: 2. For example, the inhibitory activity of the sCR1 variant of the present disclosure in the classical complement pathway is increased by at least 1.25 fold, or about 1.5 fold, or about 1.75 fold, or about 2 fold, or about 2.5 fold, or about 3 fold, or about 3.5 fold, or about 4 fold, or about 5 fold compared to a sCR1 comprising a sequence set forth in SEQ ID NO: 2.

In one example, the sCR1 variant of the present disclosure has an IC50 in a classical complement assay (e.g., Wieslab complement assay) that is less than a sCR1 comprising a sequence set forth in SEQ ID NO: 2. For example, the sCR1 variant of the present disclosure has an IC₅₀ in a classical complement assay (e.g., Wieslab complement assay) of less than about 1.0 nM, such as about 0.95 nM, or about 0.90 nM, or about 0.85 nM, or about 0.80 nM, or about 0.75 nM, or about 0.70 nM. In one example, the sCR1 variant of the present disclosure has an IC₅₀ in a classical complement assay (e.g., Wieslab complement assay) of between about 0.85 nM and 0.90 nM, such as about 0.88 nM. In one example, the sCR1 variant of the present disclosure has an IC₅₀ in a classical complement assay (e.g., Wieslab complement assay) of less than about 0.65 nM, or about 0.60 nM, or about 0.55 nM, or about 0.50 nM, or about 0.45 nM, or about 0.40 nM, or about 0.35 nM, or about 0.30 nM, or about 0.25 nM, or about 0.20 nM, or about 0.15 nM, or about 0.10 nM. In one example, the sCR1 variant of the present disclosure has an IC₅₀ in a classical complement assay (e.g., Wieslab complement assay) of between about 0.35 nM and 0.45 nM, such as about 0.40 nM.

In one example, the sCR1 conjugate has increased complement inhibitory activity in the classical pathway, the lectin pathway and/or alternative complement pathway compared to an unconjugated sCR1 variant of the present disclosure.

In one example, the sCR1 conjugate of the present disclosure has increased inhibitory activity in the classical complement pathway compared to an unconjugated sCR1 variant of the present disclosure. For example, the inhibitory activity of the sCR1 conjugate of the present disclosure in the classical complement pathway is increased by at least 1.25 fold, or about 1.5 fold, or about 1.75 fold, or about 2 fold, or about 2.5 fold, or about 3 fold, or about 3.5 fold, or about 4 fold, or about 5 fold compared to an unconjugated sCR1 variant of the present disclosure.

In one example, the sCR1 conjugate of the present disclosure has an IC₅₀ in a classical complement assay (e.g., Wieslab complement assay) that is less than an unconjugated sCR1 variant of the present disclosure. In one example, the sCR1 conjugate of the present disclosure has an IC₅₀ in a classical complement assay (e.g., Wieslab complement assay) of less than about 1.0 nM, such as about 0.95 nM, or about 0.90 nM, or about 0.85 nM, or about 0.80 nM, or about 0.75 nM, or about 0.70 nM, or about 0.65 nM, or about 0.60 nM. In one example, the sCR1 conjugate of the present disclosure has an IC₅₀ in a classical complement assay (e.g., Wieslab complement assay) of between about 0.75 nM and 0.80 nM, such as about 0.78 nM. In one example, the sCR1 conjugate of the present disclosure has an IC₅₀ in a classical complement assay (e.g., Wieslab complement assay) of between about 0.60 nM and 0.65 nM, such as about 0.63 nM. In one example, the sCR1 conjugate of the present disclosure has an IC₅₀ in a classical complement assay 15 (e.g., Wieslab complement assay) of less than about 0.65 nM, or about 0.60 nM, or about 0.55 nM, or about 0.50 nM, or about 0.45 nM, or about 0.40 nM, or about 0.35 nM, or about 0.30 nM, or about 0.25 nM, or about 0.20 nM, or about 0.15 nM, or about 0.10 nM. For example, about 0.48 nM.

In one example, the sCR1 variant has increased inhibitory activity in the lectin complement pathway compared to a sCR1 comprising a sequence set forth in SEQ ID NO: 2. For example, the inhibitory activity of the sCR1 variant of the present disclosure in the lectin complement pathway is increased by at least 1.25 fold, or about 1.5 fold, or about 1.75 fold, or about 2 fold, or about 2.5 fold, or about 3 fold, or about 3.5 fold, or about 4 fold, or about 5 fold compared to a sCR1 comprising a sequence set forth in SEQ ID NO: 2.

In one example, the sCR1 variant of the present disclosure has an IC₅₀ in a lectin complement assay (e.g., Wieslab complement assay) that is less than a sCR1 comprising a sequence set forth in SEQ ID NO: 2. For example, the sCR1 variant of the present disclosure has an IC₅₀ in a lectin complement assay (e.g., Wieslab complement assay) of less than about 0.60 nM, or about 0.55 nM, or about 0.50 nM. In one example, the sCR1 variant of the present disclosure has an IC₅₀ in a lectin complement assay (e.g., Wieslab complement assay) of between about 0.50 nM and 0.60 nM, such as about 0.547 nM. In one example, the sCR1 variant of the present disclosure has an IC₅₀ in a lectin complement assay (e.g., Wieslab complement assay) of less than about 0.50 nM, or about 0.45 nM, or about 0.40 nM, or about 0.35 nM, or about 0.30 nM. In one example, the sCR1 variant of the present disclosure has an IC₅₀ in a lectin complement assay (e.g., Wieslab complement assay) of between about 0.40 nM and 0.45 nM, such as about 0.43 nM.

In one example, the sCR1 conjugate of the present disclosure has increased inhibitory activity in the lectin complement pathway compared to an unconjugated sCR1 variant of the present disclosure. For example, the inhibitory activity of the sCR1 conjugate of the present disclosure in the lectin complement pathway is increased by at least 1.25 fold, or about 1.5 fold, or about 1.75 fold, or about 2 fold, or about 2.5 fold, or about 3 fold, or about 3.5 fold, or about 4 fold, or about 5 fold compared to an unconjugated sCR1 variant of the present disclosure.

In one example, the sCR1 conjugate of the present disclosure has an IC₅₀ in a lectin complement assay (e.g., Wieslab complement assay) that is less than an unconjugated sCR1 variant of the present disclosure. In one example, the sCR1 conjugate of the present disclosure has an IC₅₀ in a lectin complement assay (e.g., Wieslab complement assay) of less than about 0.70 nM, or about 0.60 nM, or about 0.55 nM, or about 0.50 nM, or about 0.45 nM. In one example, the sCR1 variant of the present disclosure has an IC₅₀ in a lectin complement assay (e.g., Wieslab complement assay) of between about 0.50 nM and 0.60 nM, such as about 0.53 nM or about 0.55 nM. In one example, the sCR1 variant of the present disclosure has an IC₅₀ in a lectin complement assay (e.g., Wieslab complement assay) of between about 0.45 nM and 0.50 nM, such as about 0.46 nM. In one example, the sCR1 variant of the present disclosure has an IC₅₀ in a lectin complement assay (e.g., Wieslab complement assay) of less than about 0.50 nM, or about 0.45 nM, or about 0.40 nM, or about 0.35 nM, or about 0.30 nM, such as about 0.37 nM.

In one example, the sCR1 variant has increased inhibitory activity in the alternative complement pathway compared to a sCR1 comprising a sequence set forth in SEQ ID NO: 2. For example, the inhibitory activity of the sCR1 variant of the present disclosure in the alternative complement pathway is increased by at least 1.25 fold, or about 1.5 fold, or about 1.75 fold, or about 2 fold, or about 2.5 fold, or about 3 fold, or about 3.5 fold, or about 4 fold, or about 5 fold compared to a sCR1 comprising a sequence set forth in SEQ ID NO: 2.

In one example, the sCR1 variant of the present disclosure has an IC₅₀ in an alternative complement assay (e.g., Wieslab complement assay) that is less than a sCR1 comprising a sequence set forth in SEQ ID NO: 2. For example, the sCR1 variant of the present disclosure has an IC₅₀ in an alternative complement assay (e.g., Wieslab complement assay) of less than about 0.75 nM, or about 0.70 nM, or about 0.65 nM, or about 0.60 nM, or about 0.55 nM, or about 0.50 nM, or about 0.45 nM, or about 0.40 nM, or about 0.35 nM, or about 0.30 nM, or about 0.25 nM. In one example, the sCR1 variant of the present disclosure has an IC₅₀ in an alternative complement assay (e.g., Wieslab complement assay) of between about 0.35 nM and about 0.40 nM, such as about 0.38 nM. In one example, the sCR1 variant of the present disclosure has an IC₅₀ in an alternative complement assay (e.g., Wieslab complement assay) of between about 0.25 nM and about 0.30 nM, such as about 0.27 nM.

In one example, the sCR1 conjugate of the present disclosure has increased inhibitory activity in the alternative complement pathway compared to an unconjugated sCR1 variant of the present disclosure. For example, the inhibitory activity of the sCR1 conjugate of the present disclosure in the alternative complement pathway is increased by at least 1.25 fold, or about 1.5 fold, or about 1.75 fold, or about 2 fold, or about 2.5 fold, or about 3 fold, or about 3.5 fold, or about 4 fold, or about 5 fold compared to an unconjugated sCR1 variant of the present disclosure.

In one example, the sCR1 conjugate of the present disclosure has an IC₅₀ in an alternative complement assay (e.g., Wieslab complement assay) that is less than an unconjugated sCR1 variant of the present disclosure. In one example, the sCR1 conjugate of the present disclosure has an IC₅₀ in an alternative complement assay (e.g., Wieslab complement assay) of less than about 0.75 nM, or about 0.70 nM, or about 0.65 nM, or about 0.60 nM, or about 0.55 nM, or about 0.50 nM, or about 0.45 nM, or about 0.40 nM, or about 0.35 nM, or about 0.30 nM, or about 0.25 nM. In one example, the sCR1 variant of the present disclosure has an IC₅₀ in an alternative complement assay (e.g., Wieslab complement assay) of between about 0.35 nM and about 0.40 nM, such as about 0.368 nM. In one example, the sCR1 variant of the present disclosure has an IC₅₀ in an alternative complement assay (e.g., Wieslab complement assay) of between about 0.45 nM and about 0.50 nM, such as about 0.479 nM. In one example, the sCR1 conjugate of the present disclosure has an IC₅₀ in an alternative complement assay (e.g., Wieslab complement assay) of less than about 75 pM, or about 70 pM, or about 65 pM, or about 60 pM, or about 55 pM, or about 50 pM, or about 45 pM, or about 40 pM, or about 35 pM, or about 30 pM, or about 25 pM. In one example, the sCR1 variant of the present disclosure has an IC₅₀ in an alternative complement assay (e.g., Wieslab complement assay) of between about 50 pM and about 55 pM, such as about 50.22 pM or about 53.55 pM.

In one example, the sCR1 variant of the present disclosure comprises long homologous repeat (LHR) regions selected from the group consisting of:

(i) LHR-A and LHR-B;

(ii) LHR-A, LHR-B and LHR-C;

(iii) LHR-B and LHR-C; and

(iv) LHR-B, LHR-C and LHR-D.

In one example, the sCR1 variant of the present disclosure comprises LHR regions consisting of LHR-A and LHR-B, but lacking LHR-C and LHR-D.

In one example, the sCR1 variant of the present disclosure comprises LHR regions consisting of LHR-A, LHR-B and LHR-C, but lacking LHR-D.

In one example, the sCR1 variant of the present disclosure comprises LHR regions consisting of LHR-B and LHR-C, but lacking LHR-A and LHR-D.

In one example, the sCR1 variant of the present disclosure comprises LHR regions consisting of LHR-B, LHR-C and LHR-D, but lacking LHR-A.

In one example, LHR region LHR-A comprises an amino acid sequence corresponding to amino acids 42 to 489 of SEQ ID NO: 1. For example, the LHR-A region comprises an amino acid sequence set forth in SEQ ID NO: 13. In one example, LHR region LHR-A comprises short consensus repeat (SCR) sequences 1 to 7. For example, SCR sequences 1 to 3 (i.e., Site 1) are capable of binding to C4b.

In one example, LHR region LHR-B comprises an amino acid sequence corresponding to amino acids 490 to 939 of SEQ ID NO: 1. For example, the LHR-B region comprises an amino acid sequence set forth in SEQ ID NO: 14. In one example, LHR region LHR-B comprises SCR sequences 8 to 14. For example, SCR sequences 8 to 10 (i.e., Site 2) are capable of binding to C3b and C4b.

In one example, LHR region LHR-C comprises an amino acid sequence corresponding to amino acids 940 to 1392 of SEQ ID NO: 1. For example, the LHR-C region comprises an amino acid sequence set forth in SEQ ID NO: 15. In one example, LHR region LHR-C comprises SCR sequences 15 to 21. For example, SCR sequences 15 to 17 are capable of binding to C3b and C4b.

In one example, LHR region LHR-D comprises an amino acid sequence corresponding to amino acids 1393 to 1971 of SEQ ID NO: 1. For example, the LHR-D region comprises an amino acid sequence set forth in SEQ ID NO: 16. In one example, LHR region LHR-D comprises SCR sequences 22 to 28. For example, SCR sequences 22 to 28 are capable of binding to C1q and MBL.

In one example, the sCR1 variant of the present disclosure comprises (or consists of) SCR sequences selected from the group consisting of:

(i) SCR-1 to SCR-14 (e.g., lacking SCR-15 to SCR-28);

(ii) SCR-1 to SCR-21 (e.g., lacking SCR-22 to SCR-28);

(iii) SCR-8 to SCR-21 (e.g., lacking SCR-1 to SCR-7 and SCR-22 to SCR-28); and

(iv) SCR-8 to SCR-28 (e.g., lacking SCR-1 to SCR-7).

In one example, the sCR1 variant of the present disclosure comprises SCR sequences SCR-1 to SCR-14 (e.g., lacking SCR-15 to SCR-28).

In one example, the sCR1 variant of the present disclosure comprises SCR sequences SCR-1 to SCR-21 (e.g., lacking SCR-22 to SCR-28).

In one example, the sCR1 variant of the present disclosure comprises SCR sequences SCR-8 to SCR-21 (e.g., lacking SCR-1 to SCR-7 and SCR-22 to SCR-28).

In one example, the sCR1 variant of the present disclosure comprises SCR sequences SCR-8 to SCR-28 (e.g., lacking SCR-1 to SCR-7).

In one example, the sCR1 variant is monomeric (i.e., one copy of the sCR1 variant).

In one example, the sCR1 variant is dimeric, or dimerized (i.e., two copies of a sCR1 variant are linked in a fusion protein).

In one example, the sCR1 variant is multimeric, or multimerized (i.e., multiple copies of a sCR1 variant are linked in a fusion protein).

In one example, two or more of the same sCR1 variant are fused (i.e., expressed as a fusion protein).

In one example, two or more different sCR1 variants are fused (i.e., expressed as a fusion protein).

In one example, the dimerized or multimerized sCR1 variant comprises a linker between the sCR1 variants.

In one example, the disclosure provides a multimeric protein comprising two or more sCR1 variants comprising a multimerization domain, wherein the multimerization domains interact to form the multimeric protein.

In one example, each sCR1 variant in the multimeric protein comprises one sCR1 variant. In another example, one or more sCR1 variants in the multimeric protein comprises two or more sCR1 variants, e.g., the sCR1 variants are linked in a fusion protein.

In one example, the multimerization domain comprises an immunoglobulin hinge domain.

In one example, the multimerization domain is a leucine zipper domain, a cystine knot or an antibody Fc region.

In one example, the multimerized sCR1 variant is linear.

In one example, the multimerized sCR1 variant is circular.

The present disclosure provides a sCR1 conjugate as described herein in any example (e.g., the description of sCR1 variants shall be taken to apply to the following description in relation to sCR1 conjugates per se) conjugated to a protein comprising an antigen binding domain that binds to a target and inhibits signaling by or via the target.

The present disclosure also provides a sCR1 conjugate as described herein in any example (e.g., the description of sCR1 variants shall be taken to apply to the following description in relation to sCR1 conjugates per se) conjugated to a protein comprising an antigen binding domain that binds to a blood coagulation factor.

In one example, the sCR1 variant is chemically conjugated to the protein comprising an antigen binding domain In another example, the sCR1 variant is fused, e.g., expressed as a fusion protein, with the protein comprising an antigen binding domain In one example, the protein comprising an antigen binding domain is conjugated to the C-terminus of the sCR1 variant. In one example, the protein comprising an antigen binding domain is conjugated to the N-terminus of the sCR1 variant.

In one example, the sCR1 variant is conjugated to a protein comprising an antigen binding domain, wherein antigen binding domain binds to or specifically binds to the target and neutralises the signalling.

In one example, the protein neutralizes G-CSF signalling.

In one example, the target is granulocyte colony stimulating factor (G-CSF) or G-CSF receptor (G-CSFR).

In one example, the protein that neutralizes G-CSF signaling binds to or specifically binds to G-CSF or to G-CSF receptor (G-CSFR). In one example, the protein that neutralizes G-CSF signaling binds to or specifically binds to G-CSF. In one example, the protein that neutralizes G-CSF signaling binds to or specifically binds to G-CSF receptor (G-CSFR).

In one example, the protein binds to or specifically binds to G-CSF and neutralizes G-CSF signalling. Reference herein to a protein or antibody that “binds to” G-CSF provides literal support for a protein or antibody that “binds specifically to” G-CSF.

In one example, the protein binds to or specifically binds to G-CSFR and neutralizes G-CSF signalling. Reference herein to a protein or antibody that “binds to” G-CSFR provides literal support for a protein or antibody that “binds specifically to” G-CSFR.

In one example, the sCR1 variant is conjugated to a protein comprising an antigen binding domain, wherein the antigen binding domain binds to or specifically binds to the blood coagulation factor and antagonises activity and/or antagonises activation of the blood coagulation factor.

In one example, the blood coagulation factor is selected from the group consisting of Factor I, Factor II (prothrombin)/thrombin, Factor III, Factor V, Factor VII, Factor VIII, Factor IX, Factor X, Factor XI, Factor XII, Factor XIII and an activated form of any of the foregoing. For example, the blood coagulation factor is Factor XII and/or activated Factor XII (Factor XIIa). In another example, the blood coagulation factor is Factor XI and/or activated Factor XI (Factor XIa).

In one example, the protein that binds to Factor XII/XIIa binds to or specifically binds to Factor XII or to activated Factor XII (Factor XIIa). In one example, the protein that binds to Factor XII/XIIa binds to or specifically binds to Factor XII. In one example, the protein that binds to Factor XII/XIIa binds to or specifically binds to activated Factor XII (Factor XIIa).

In one example, the protein binds to or specifically binds to Factor XII and antagonises activation of Factor XII/XIIa and/or antagonises activity of Factor XII/XIIa. Reference herein to a protein or antibody that “binds to” Factor XII provides literal support for a protein or antibody that “binds specifically to” Factor XII.

In one example, the protein binds to or specifically binds to activated Factor XII (Factor XIIa) and antagonises activation of Factor XII/XIIa and/or antagonises activity of Factor XII/XIIa. Reference herein to a protein or antibody that “binds to” activated Factor XII provides literal support for a protein or antibody that “binds specifically to” activated Factor XII.

In one example, the protein that binds to Factor XI/XIa binds to or specifically binds to Factor XI or to activated Factor XI (Factor XIa). In one example, the protein that binds to Factor XI/XIa binds to or specifically binds to Factor XI. In one example, the protein that binds to Factor XI/XIa binds to or specifically binds to activated Factor XI (Factor XIa).

In one example, the protein binds to or specifically binds to Factor XI and neutralizes Factor XI/XIa activity. Reference herein to a protein or antibody that “binds to” Factor XI provides literal support for a protein or antibody that “binds specifically to” Factor XI.

In one example, the protein binds to or specifically binds to activated Factor XI (Factor XIa) and antagonises activation of Factor XI/XIa and/or antagonises activity of Factor XI/XIa. Reference herein to a protein or antibody that “binds to” activated Factor XI provides literal support for a protein or antibody that “binds specifically to” activated Factor XI.

In one example, the protein comprises an antigen binding domain of an antibody. For example, the protein comprises at least a V_{H} and a V_{L}, wherein the V_{H} and V_{L} bind to form a Fv comprising an antigen binding domain.

In one example, the V_{H} and the V_{L} are in a single polypeptide chain. For example, the protein is:
(i) a single chain Fv fragment (scFv);
(ii) a dimeric scFv (di-scFv); or
(iii) at least one of (i) and/or (ii) linked to a constant region of an antibody, Fc or a heavy chain constant domain (C_{H}) 2 and/or C_{H}3.

In one example, the V_{L} and V_{H} are in separate polypeptide chains. For example, the protein is:
(i) a diabody;
(ii) a triabody;
(iii) a tetrabody;
(iv) a Fab;
(v) a F(ab′)₂;
(vi) a Fv; or
(vii) one of (i) to (vi) linked to a constant region of an antibody, Fc or a heavy chain constant domain (C_{H}) 2 and/or C_{H}3.

The foregoing proteins (described in the previous two lists) can also be referred to as antigen binding domains of antibodies.

In one example, the protein is an antibody or antigen binding fragment thereof (e.g., a scFv comprising the variable regions of the antibody). Exemplary antibodies are full-length antibodies such as described in WO2012171057, which is incorporated herein by reference. Additional exemplary antibodies are described, for example, in WO2013014092, WO2009067660, WO2009154461, WO2010080623, WO2013167669, WO02016207858, WO2017015619, WO2017162791, WO2017127468 and WO2017218371, which are incorporated herein by reference.

In one example, the protein comprises an Fc region. For example, the Fc region is a human IgG₁ Fc region or a human IgG₄ Fc region or a stabilized human IgG₄ Fc region. For example, the Fc region is a human IgG₄ Fc region. In one example, the antibody Fc region is modified to prevent dimerization, (e.g., as discussed herein).

In one example, the sCR1 variant of the present disclosure is conjugated to an antibody. In one example, the antibody is conjugated to the N-terminus of the sCR1 variant. In another example, the antibody is conjugated to the C-terminus of the sCR1 variant. For example, the sCR1 variant consists of an amino acid sequence corresponding to amino acids 42 to 1392 of SEQ ID NO: 1 or comprises an amino acid sequence corresponding to amino acids 42 to 1392 of SEQ ID NO: 1 (e.g., lacking amino acid residues 1393 to 1971 of SEQ ID NO: 1) and an antibody is fused to the N-terminus of the sCR1 variant

In one example, the sCR1 variant is fused to the Fc region of the antibody. In one example, the C-terminus of the sCR1 variant is conjugated to the C-terminus of the Fc region of the antibody. For example, the C-terminus of the sCR1 variant is cross-linked to the C-terminus of the Fc region of the antibody. For example, the C-terminus of the sCR1 variant can comprise a cysteine residue and the C-terminus of the Fc region can comprise a cysteine residue and the cysteine residues are cross-linked.

In one example, the protein comprises a scFv. In one example, the protein comprises a scFv that binds to or specifically binds to G-CSFR and neutralizes G-CSF signalling. For example, the sCR1 variant of the present disclosure is conjugated to a scFv that binds to G-CSFR. In one example, the protein comprises a scFv that binds to or specifically binds to a blood coagulation factor (and e.g., antagonizes activity of the blood coagulation factor or antagonizes activation of the blood coagulation factor). For example, the sCR1 variant of the present disclosure is conjugated to a scFv that binds to a blood coagulation factor, e.g., Factor XII or Factor XIIa or Factor XI or Factor XIa. In one example, the protein comprises a scFv that binds to or specifically binds to Factor XII and/or activated Factor XII (Factor XIIa) and antagonises activation of Factor XII/XIIa and/or antagonises activity of Factor XII/XIIa. For example, the sCR1 variant of the present disclosure is conjugated to a scFv that binds to Factor XII. In another example, the protein comprises a scFv that binds to or specifically binds to Factor XI and/or activated Factor XI (Factor XIa) and antagonises activation of Factor XI/XIa and/or antagonises activity of Factor XI/XIa. For example, the sCR1 variant of the present disclosure is conjugated to a scFv that binds to Factor XI.

In one example, the scFv is conjugated to the N-terminus of the sCR1 variant. For example, the C-terminus of the scFv is conjugated to the N-terminus of the sCR1 variant.

In one example, the scFv is conjugated to the C-terminus of the sCR1 variant. For example, the C-terminus of the scFv is conjugated to the C-terminus of the sCR1 variant.

In another example, the N-terminus of the scFv is conjugated to the C-terminus of the sCR1 variant. For example, the N-terminus of the V_{H} of the scFv is conjugated to the C-terminus of the sCR1 variant. For example, the N-terminus of the V_{L} of the scFv is conjugated to the C-terminus of the sCR1 variant.

In one example, the protein comprising an antigen binding domain that binds to G-CSFR is an antibody, i.e., a full-length antibody. In another example, the protein comprising an antigen binding domain that binds to a blood coagulation factor is an antibody, i.e., a full-length antibody. In one example, the sCR1 variant is conjugated to the C-terminus of the antibody heavy chain. For example, the C-terminus of the sCR1 variant is conjugated to the C-terminus of the antibody heavy chain.

For example, the C-terminus of the sCR1 variant is cross-linked to the C-terminus of the heavy chain of the antibody. For example, the C-terminus of the sCR1 variant can comprise a cysteine residue and the C-terminus of the heavy chain of the antibody can comprise a cysteine residue and the cysteine residues are cross-linked.

In another example, the sCR1 variant is conjugated to the N-terminus of the antibody heavy chain. For example, the C-terminus of the sCR1 variant is conjugated to the C-terminus of the antibody heavy chain. In one example, the sCR1 variant is fused to the C-terminus of the antibody heavy chain.

In another example, the sCR1 variant is conjugated to the N-terminus of the antibody light chain. For example, the C-terminus of the sCR1 variant is conjugated to the C-terminus of the antibody light chain. In one example, the sCR1 variant is fused to the C-terminus of the antibody light chain.

As discussed herein in relation to proteins conjugated to one another, the description shall be understood that additional components, e.g., a linker, can be included between the proteins. For example, description of conjugation of a sCR1 variant to the N-terminus of the antibody heavy chain, will be understood to mean that the sCR1 variant may be separated from the N-terminus of the antibody heavy chain by a linker, e.g., an amino acid linker.

In one example, the protein is chimeric, de-immunized, humanized, human or primatized. In one example, the protein or antibody is human

In one example, the protein comprises an antibody variable region that competitively inhibits the binding of antibody C1.2G comprising a heavy chain variable region (V_{H}) comprising a sequence set forth in SEQ ID NO: 38 and a light chain variable region (V_{L}) comprising a sequence set forth in SEQ ID NO: 39 to G-CSFR.

In one example, the protein binds to an epitope comprising residues within one or two or three or four regions selected from 111-115, 170-176, 218-234 and/or 286-300 of SEQ ID NO: 48.

In one example, the protein comprises a heavy chain variable region (VH) comprising an amino acid sequence set forth in SEQ ID NO: 36 and a light chain variable region (VL) comprising an amino acid sequence set forth in SEQ ID NO: 37.

In one example, the protein is an antibody or antigen binding fragment thereof comprising a V_{H} comprising the complementarity determining regions (CDRs) of a V_{H} comprising an amino acid sequence set forth in SEQ ID NO: 36 and a V_{L} comprising the CDRs of a V_{L} comprising an amino acid sequence set forth in SEQ ID NO: 37. For example, the protein comprises:
(i) a V_{H} comprising:
   (a) a CDR1 comprising a sequence set forth in amino acids 25-34 of SEQ ID NO: 36;
   (b) a CDR2 comprising a sequence set forth in amino acids 49-65 of SEQ ID NO: 36; and
   (c) a CDR3 comprising a sequence set forth in amino acids 98-108 of SEQ ID NO: 36; and/or
(ii) a V_{L} comprising:
   (a) a CDR1 comprising a sequence set forth in amino acids 23-33 of SEQ ID NO: 37;
   (b) a CDR2 comprising a sequence set forth in amino acids 49-55 of SEQ ID NO: 37; and
   (c) a CDR3 comprising a sequence set forth in amino acids 88-96 of SEQ ID NO: 37.

In one example, the protein comprises:
(i) a VH comprising:
   (a) a CDR1 comprising a sequence set forth in SEQ ID NO: 40;
   (b) a CDR2 comprising a sequence set forth in SEQ ID NO: 41; and
   (c) a CDR3 comprising a sequence set forth in SEQ ID NO: 42; and/or
(ii) a VL comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 45.

In one example, the protein comprises a heavy chain variable region (V_{H}) comprising an amino acid sequence set forth in SEQ ID NO: 38 and a light chain variable region (V_{L}) comprising an amino acid sequence set forth in SEQ ID NO: 39.

In one example, the protein is an antibody or antigen binding fragment thereof comprising a V_{H} comprising the CDRs of a V_{H} comprising an amino acid sequence set forth in SEQ ID NO: 38 and a V_{L} comprising the CDRs of a V_{L} comprising an amino acid sequence set forth in SEQ ID NO: 39. For example, the protein comprises:
(i) a V_{H} comprising:
   (a) a CDR1 comprising a sequence set forth in amino acids 25-34 of SEQ ID NO: 38;
   (b) a CDR2 comprising a sequence set forth in amino acids 49-65 of SEQ ID NO: 38; and
   (c) a CDR3 comprising a sequence set forth in amino acids 98-108 of SEQ ID NO: 38; and/or
(ii) a V_{L} comprising:
   (a) a CDR1 comprising a sequence set forth in amino acids 23-33 of SEQ ID NO: 39;
   (b) a CDR2 comprising a sequence set forth in amino acids 49-55 of SEQ ID NO: 39; and
   (c) a CDR3 comprising a sequence set forth in amino acids 88-96 of SEQ ID NO: 39.

In one example, the protein comprises a heavy chain variable region (V_{H}) comprising an amino acid sequence set forth in SEQ ID NO: 46 and a light chain variable region (V_{L}) comprising an amino acid sequence set forth in SEQ ID NO: 47.

In one example, the protein is an antibody or antigen binding fragment thereof comprising a V_{H} comprising the CDRs of a V_{H} comprising an amino acid sequence set forth in SEQ ID NO: 46 and a V_{L} comprising the CDRs of a V_{L} comprising an amino acid sequence set forth in SEQ ID NO: 47. For example, the protein comprises:
(i) a V_{H} comprising:
   (a) a CDR1 comprising a sequence set forth in amino acids 25-34 of SEQ ID NO: 46;
   (b) a CDR2 comprising a sequence set forth in amino acids 49-65 of SEQ ID NO: 46; and
   (c) a CDR3 comprising a sequence set forth in amino acids 98-108 of SEQ ID NO: 46; and/or
(ii) a VL comprising:
   (a) a CDR1 comprising a sequence set forth in amino acids 23-33 of SEQ ID NO: 47;
   (b) a CDR2 comprising a sequence set forth in amino acids 49-55 of SEQ ID NO: 47; and
   (c) a CDR3 comprising a sequence set forth in amino acids 88-96 of SEQ ID NO: 47.

In one example, the protein, antibody or antigen binding fragment thereof is any form of the protein, antibody or functional fragment thereof encoded by a nucleic acid 35 encoding any of the foregoing proteins, antibodies or functional fragments.

In one example, the sCR1 conjugate of the present disclosure comprises:

(i) an amino acid sequence corresponding to amino acids 42 to 1649 of SEQ ID NO: 49;

(ii) an amino acid sequence corresponding to amino acids 42 to 1649 of SEQ ID NO: 50;

(iii) an amino acid sequence corresponding to amino acids 42 to 1656 of SEQ ID NO: 51;

(iv) an amino acid sequence corresponding to amino acids 42 to 1656 of SEQ ID NO: 52;

(v) an amino acid sequence corresponding to amino acids 42 to 1648 of SEQ ID NO: 53; or

(vi) an amino acid sequence corresponding to amino acids 42 to 1648 of SEQ ID NO: 54.

In one example, the sCR1 conjugate consists of an amino acid sequence corresponding to amino acids 42 to 1649 of SEQ ID NO: 49 or comprises an amino acid sequence corresponding to amino acids 42 to 1649 of SEQ ID NO: 49 (e.g., lacking amino acid residues 1 to 41 of SEQ ID NO: 49).

In one example, the conjugated sCR1 variant consists of an amino acid sequence corresponding to amino acids 42 to 1649 of SEQ ID NO: 50 or comprises an amino acid sequence corresponding to amino acids 42 to 1649 of SEQ ID NO: 50 (e.g., lacking amino acid residues 1 to 41 of SEQ ID NO: 50).

In one example, the conjugated sCR1 variant consists of an amino acid sequence corresponding to amino acids 42 to 1656 of SEQ ID NO: 51 or comprises an amino acid sequence corresponding to amino acids 42 to 1656 of SEQ ID NO: 51 (e.g., lacking amino acid residues 1 to 41 of SEQ ID NO: 51).

In one example, the conjugated sCR1 variant consists of an amino acid sequence corresponding to amino acids 42 to 1656 of SEQ ID NO: 52 or comprises an amino acid sequence corresponding to amino acids 42 to 1656 of SEQ ID NO: 52 (e.g., lacking amino acid residues 1 to 41 of SEQ ID NO: 52).

In one example, the conjugated sCR1 variant consists of an amino acid sequence corresponding to amino acids 42 to 1648 of SEQ ID NO: 53 or comprises an amino acid sequence corresponding to amino acids 42 to 1656 of SEQ ID NO: 53 (e.g., lacking amino acid residues 1 to 41 of SEQ ID NO: 53).

In one example, the conjugated sCR1 variant consists of an amino acid sequence corresponding to amino acids 42 to 1656 of SEQ ID NO: 54 or comprises an amino acid sequence corresponding to amino acids 42 to 1656 of SEQ ID NO: 54 (e.g., lacking amino acid residues 1 to 41 of SEQ ID NO: 54).

In one example, the protein comprises an antibody variable region that competitively inhibits the binding of antibody 3F7 comprising a heavy chain variable region (V_{H}) comprising a sequence set forth in SEQ ID NO: 56 and a light chain variable region (V_{L}) comprising a sequence set forth in SEQ ID NO: 57 to Factor XII.

In one example, the protein comprises an antibody variable region that competitively inhibits the binding of germlined antibody 3F7 (3F7G) comprising a heavy chain variable region (V_{H}) comprising a sequence set forth in SEQ ID NO: 58 and a light chain variable region (V_{L}) comprising a sequence set forth in SEQ ID NO: 59 to Factor XII.

In one example, the protein comprises an antibody variable region that competitively inhibits the binding of affinity matured antibody 3F7 (3F7aff) comprising a heavy chain variable region (V_{H}) comprising a sequence set forth in SEQ ID NO: 60 and a light chain variable region (VL) comprising a sequence set forth in SEQ ID NO: 61 to Factor XII.

In one example, the protein comprises a heavy chain variable region (VH) comprising an amino acid sequence set forth in SEQ ID NO: 56 and a light chain variable region (V_{L}) comprising an amino acid sequence set forth in SEQ ID NO: 57.

In one example, the protein is an antibody or antigen binding fragment thereof comprising a V_{H} comprising the complementarity determining regions (CDRs) of a V_{H} comprising an amino acid sequence set forth in SEQ ID NO: 56 and a VL comprising the CDRs of a V_{L} comprising an amino acid sequence set forth in SEQ ID NO: 57. For example, the protein comprises:
(i) a V_{H} comprising:
   (a) a CDR1 comprising a sequence set forth in amino acids 25-34 of SEQ ID NO: 56;
   (b) a CDR2 comprising a sequence set forth in amino acids 49-65 of SEQ ID NO: 56; and
   (c) a CDR3 comprising a sequence set forth in amino acids 98-108 of SEQ ID NO: 56; and/or
(ii) a V_{L} comprising:
   (a) a CDR1 comprising a sequence set forth in amino acids 23-33 of SEQ ID NO: 57;
   (b) a CDR2 comprising a sequence set forth in amino acids 49-55 of SEQ ID NO: 57; and
   (c) a CDR3 comprising a sequence set forth in amino acids 88-96 of SEQ ID NO: 57.

In one example, the protein comprises a heavy chain variable region (V_{H}) comprising an amino acid sequence set forth in SEQ ID NO: 58 and a light chain variable region (V_{L}) comprising an amino acid sequence set forth in SEQ ID NO: 59.

In one example, the protein is an antibody or antigen binding fragment thereof comprising a V_{H} comprising the complementarity determining regions (CDRs) of a V_{H} comprising an amino acid sequence set forth in SEQ ID NO: 58 and a V_{L} comprising the CDRs of a V_{L} comprising an amino acid sequence set forth in SEQ ID NO: 59. For example, the protein comprises:
(i) a VH comprising:
   (a) a CDR1 comprising a sequence set forth in amino acids 25-34 of SEQ ID NO: 58;
   (b) a CDR2 comprising a sequence set forth in amino acids 49-65 of SEQ ID NO: 58; and
   (c) a CDR3 comprising a sequence set forth in amino acids 98-108 of SEQ ID NO: 58; and/or
(ii) a VL comprising:
   (a) a CDR1 comprising a sequence set forth in amino acids 23-33 of SEQ ID NO: 59;
   (b) a CDR2 comprising a sequence set forth in amino acids 49-55 of SEQ ID NO: 59; and
   (c) a CDR3 comprising a sequence set forth in amino acids 88-96 of SEQ ID NO: 59.

In one example, the protein comprises a heavy chain variable region (V_{H}) comprising an amino acid sequence set forth in SEQ ID NO: 60 and a light chain variable region (V_{L}) comprising an amino acid sequence set forth in SEQ ID NO: 61.

In one example, the protein is an antibody or antigen binding fragment thereof comprising a V_{H} comprising the complementarity determining regions (CDRs) of a V_{H} comprising an amino acid sequence set forth in SEQ ID NO: 60 and a V_{L} comprising the CDRs of a V_{L} comprising an amino acid sequence set forth in SEQ ID NO: 61. For example, the protein comprises:
(i) a V_{H} comprising:
   (a) a CDR1 comprising a sequence set forth in amino acids 25-34 of SEQ ID NO: 60;
   (b) a CDR2 comprising a sequence set forth in amino acids 49-65 of SEQ ID NO: 60; and
   (c) a CDR3 comprising a sequence set forth in amino acids 98-108 of SEQ ID NO: 60; and/or
(ii) a V_{L} comprising:
   (a) a CDR1 comprising a sequence set forth in amino acids 23-33 of SEQ ID NO: 61;
   (b) a CDR2 comprising a sequence set forth in amino acids 49-55 of SEQ ID NO: 61; and
   (c) a CDR3 comprising a sequence set forth in amino acids 88-96 of SEQ ID NO: 61.

In one example, the protein, antibody or antigen binding fragment thereof is any form of the protein, antibody or functional fragment thereof encoded by a nucleic acid encoding any of the foregoing proteins, antibodies or functional fragments.

In one example, the sCR1 conjugate of the present disclosure comprises:
(i) an amino acid sequence corresponding to amino acids 42 to 1663 of SEQ ID NO: 62;
(ii) an amino acid sequence corresponding to amino acids 42 to 1663 of SEQ ID NO: 63; or
(iii) an amino acid sequence corresponding to amino acids 42 to 1663 of SEQ ID NO: 64.

In one example, the sCR1 conjugate consists of an amino acid sequence corresponding to amino acids 42 to 1663 of SEQ ID NO: 62 or comprises an amino acid sequence corresponding to amino acids 42 to 1663 of SEQ ID NO: 62 (e.g., lacking amino acid residues 1 to 41 of SEQ ID NO: 62).

In one example, the sCR1 conjugate consists of an amino acid sequence corresponding to amino acids 42 to 1663 of SEQ ID NO: 63 or comprises an amino acid sequence corresponding to amino acids 42 to 1663 of SEQ ID NO: 63 (e.g., lacking amino acid residues 1 to 41 of SEQ ID NO: 63).

In one example, the sCR1 conjugate consists of an amino acid sequence corresponding to amino acids 42 to 1663 of SEQ ID NO: 64 or comprises an amino acid sequence corresponding to amino acids 42 to 1663 of SEQ ID NO: 64 (e.g., lacking amino acid residues 1 to 41 of SEQ ID NO: 64).

In one example, the present disclosure provides a soluble complement receptor type 1 (sCR1) conjugate comprising:
(i) an sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising a scFv that binds to or specifically binds to Factor XII and activated Factor XII (FXIIa).

In another example, the present disclosure provides a soluble complement receptor type 1 (sCR1) conjugate comprising:
(i) an sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising a scFv that binds to or specifically binds to Factor XII or activated Factor XII (FXIIa).

In one example, the present disclosure provides a soluble complement receptor type 1 (sCR1) conjugate comprising:
(i) an sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising a scFv that binds to or specifically binds to Factor XI and activated Factor XI (FXIa).

In another example, the present disclosure provides a soluble complement receptor type 1 (sCR1) conjugate comprising:
(i) an sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising a scFv that binds to or specifically binds to Factor XI or activated Factor XI (FXIa).

In one example, the sCR1 conjugate of the present disclosure has a longer serum half-life compared to a sCR1 conjugate comprising a sCR1 set forth in SEQ ID NO: 2. Examples of increased serum half-life and assays for determining serum half-life are described herein and are to be taken to apply mutatis mutandis to this example of the disclosure.

The present disclosure also provides a composition comprising a sCR1 conjugate of the disclosure and a pharmaceutical carrier and/or excipient.

The present disclosure provides a method of inhibiting complement activity in a subject, the method comprising administering the sCR1 conjugate of the present disclosure, or the composition comprising the sCR1 conjugate.

The present disclosure also provides a method of inhibiting G-CSF activity in a subject, the method comprising administering the sCR1 conjugate of the present disclosure, or the composition comprising the sCR1 conjugate.

In one example, the present disclosure provides a method of inhibiting complement activity and G-CSF activity in a subject.

The present disclosure also provides a method of treating or preventing a disease or condition in a subject, the method comprising administering the sCR1 conjugate of the present disclosure, or the composition comprising the sCR1 conjugate.

In one example, the present disclosure provides a sCR1 conjugate, or a composition comprising the sCR1 conjugate, for use in inhibiting complement activity and G-CSF activity in a subject. For example, the present disclosure provides a sCR1 conjugate, or a composition comprising the sCR1 conjugate, for use in inhibiting complement activity in a subject. In another example, present disclosure provides a sCR1 conjugate, or a composition comprising the sCR1 conjugate, for use in inhibiting G-CSF activity in a subject.

In one example, the present disclosure provides a sCR1 conjugate, or a composition comprising the sCR1 conjugate, for use in inhibiting complement activity and/or antagonising activity of Factor XII/XIIa and/or antagonising activation of Factor XII/XIIa in a subject. In one example, the present disclosure provides a sCR1 conjugate, or a composition comprising the sCR1 conjugate, for use in inhibiting complement activity and/or antagonising activity of Factor XI/XIa and/or antagonising activation of Factor XI/XIa in a subject. For example, the present disclosure provides a sCR1 conjugate, or a composition comprising the sCR1 conjugate, for use in inhibiting complement activity in a subject. In another example, present disclosure provides a sCR1 conjugate, or a composition comprising the sCR1 conjugate, for use in antagonising activation of Factor XII/XIIa in a subject. In another example, present disclosure provides a sCR1 conjugate, or a composition comprising the sCR1 conjugate, for use in antagonising activity of Factor XII/XIIa in a subject. In a further example, the present disclosure provides a sCR1 conjugate, or a composition comprising the sCR1 conjugate, for use in antagonising activation of Factor XI/XIa in a subject. In a further example, the present disclosure provides a sCR1 conjugate, or a composition comprising the sCR1 conjugate, for use in antagonising activity of Factor XI/XIa in a subject.

In one example, the present disclosure provides a sCR1 conjugate, or a composition comprising the sCR1 conjugate, for use in treating or preventing a disease or condition in a subject.

In one example, the present disclosure provides a use of the sCR1 conjugate, or the composition comprising the sCR1 conjugate of the present disclosure, in the manufacture of a medicament for inhibiting complement activity and G-CSF activity in a subject. For example, the present disclosure provides a use of the sCR1 conjugate, or the composition comprising the sCR1 conjugate of the present disclosure, in the manufacture of a medicament for inhibiting complement activity in a subject. In another example, the present disclosure provides a use of the sCR1 conjugate, or the composition comprising the sCR1 conjugate of the present disclosure, in the manufacture of a medicament for inhibiting G-CSF activity in a subject.

In one example, the present disclosure provides a use of the sCR1 conjugate, or the composition comprising the sCR1 conjugate of the present disclosure, in the manufacture of a medicament for inhibiting complement activity and/or antagonising activity of Factor XII/XIIa and/or antagonising activation of Factor XII/XIIa in a subject. In another example, the present disclosure provides a use of the sCR1 conjugate, or the composition comprising the sCR1 conjugate of the present disclosure, in the manufacture of a medicament for inhibiting complement activity and/or antagonising activity of Factor XI/XIa and/or antagonising activation of Factor XI/XIa in a subject. For example, the present disclosure provides a use of the sCR1 conjugate, or the composition comprising the sCR1 conjugate of the present disclosure, in the manufacture of a medicament for inhibiting complement activity in a subject. In another example, the present disclosure provides a use of the sCR1 conjugate, or the composition comprising the sCR1 conjugate of the present disclosure, in the manufacture of a medicament for antagonising activity of Factor XII/XIIa and/or antagonising activation of Factor XII/XIIa in a subject. In a further example, the present disclosure provides a use of the sCR1 conjugate, or the composition comprising the sCR1 conjugate of the present disclosure, in the manufacture of a medicament for antagonising activity of Factor XI/XIa and/or antagonising activation of Factor XI/XIa in a subject.

In one example, the present disclosure provides a use of the sCR1 conjugate, or the composition comprising the sCR1 conjugate of the present disclosure, in the manufacture of a medicament for the treatment or prevention of a disease or condition in a subject.

In one example, the subject is in need of treatment with a sCR1 conjugate of the present disclosure (i.e., in need thereof).

In one example, the disease or condition is a complement-mediated disorder, a neutrophil-mediated disorder, and/or a blood coagulation disorder. For example, the subject is suffering from, or at risk of a complement-mediated disorder, a neutrophil-mediated disorder and/or a blood coagulation disorder.

In one example, the subject suffers from a complement-mediated disorder, a neutrophil-mediated disorder and/or a blood coagulation disorder.

In one example, the subject has been diagnosed as suffering from a complement-mediated disorder, a neutrophil-mediated disorder and/or a blood coagulation disorder. In one example, the subject is receiving treatment for a complement-mediated disorder, a neutrophil-mediated disorder and/or a blood coagulation disorder.

In one example of any method described herein, the sCR1 conjugate or composition comprising the sCR1 conjugate of the present disclosure is administered before or after the development of a complement-mediated disorder, a neutrophil-mediated disorder and/or a blood coagulation disorder. In one example of any method described herein, the sCR1 conjugate or composition comprising the sCR1 conjugate of the present disclosure is administered before the development of the complement-mediated disorder, the neutrophil-mediated disorder and/or the blood coagulation disorder. In one example of any method described herein, the sCR1 conjugate or composition comprising the sCR1 conjugate of the present disclosure is administered after the development of the complement-mediated disorder, the neutrophil-mediated disorder and/or the blood coagulation disorder.

In one example, the subject is at risk of developing a complement-mediated disorder, a neutrophil-mediated disorder and/or a blood coagulation disorder.

In one example, the sCR1 conjugate or composition comprising the sCR1 conjugate is administered before or after the onset of symptoms of a complement-mediated disorder, a neutrophil-mediated disorder and/or a blood coagulation disorder. In one example, the sCR1 conjugate or composition comprising the sCR1 conjugate is administered before the onset of symptoms of a complement-mediated disorder, a neutrophil-mediated disorder and/or a blood coagulation disorder. In one example, the sCR1 conjugate or composition comprising the sCR1 conjugate is administered after the onset of symptoms of a complement-mediated disorder, a neutrophil-mediated disorder and/or a blood coagulation disorder. In one example, the sCR1 conjugate or composition comprising the sCR1 conjugate of the present disclosure is administered at a dose that alleviates or reduces one or more of the symptoms of a complement-mediated disorder, a neutrophil-mediated disorder and/or a blood coagulation disorder.

Symptoms of a complement-mediated disorder, a neutrophil-mediated disorder and/or a blood coagulation disorder will be apparent to the skilled person and will be dependent on the condition. Exemplary symptoms of a complement-mediated disorder, a neutrophil-mediated disorder and/or a blood coagulation disorder include, for example
Recurring infection;
Joint inflammation;
Muscle weakness;
Rash or discolouration of the skin;
Edema, especially in the extremities (e.g., feet, hands, legs or arms) or eyes;
Abdominal pain;
Breathing difficulties (e.g., wheezing, breathlessness, chest tightness, and/or coughing);
Nausea;
Fatigue;
Hematuria;
Ulceration
Partial or complete paralysis;
Poor cognitive ability;
Pain, swelling and tenderness in the affected area;
A dull or heavy ache in the affected area;
Warm skin in the area of a thrombus;
Red skin;
Chest pain; and
Sudden loss of strength in one arm or leg.

In one example, the complement-mediated disorder, the neutrophil-mediated disorder and/or the blood coagulation disorder is caused by primary dysregulation of the complement system, an autoimmune disorder, an acute injury, cancer (including metastasis) and/or an inflammatory condition. For example, the complement-mediated disorder, the neutrophil-mediated disorder and/or the blood coagulation disorder is selected from the group consisting of an inflammatory joint condition, inflammatory arthritis, inflammatory eye condition, inflammatory lung condition, inflammatory neurological condition, autoimmune intestinal disorders, psoriasis, cancer (including angiogenesis thereof) or metastasis thereof, solid organ transplantation (e.g., lung and/or renal transplantation (including antibody mediated rejection)), ischemia reperfusion injury before, during or after transplantation, delayed graft function, asthma and exacerbated forms thereof, neutrophilic dermatosis, a neutrophilic skin lesion, ischemic stroke with reperfusion, neurotraumatic disorder, somatic trauma, ischemia-reperfusion injury (IRI, including myocardial IRI, intestinal IRI, liver IRI and/or pancreatic IRI), venous, arterial or capillary thrombus formation, thrombus formation in the heart, contact-mediated thrombo-inflammation, thrombus formation during and/or after contacting blood of a human or animal subject with artificial surfaces, interstitial lung disease, inflammation, a neurological inflammatory disease, fibrinolysis, angiogenesis, a thrombo-inflammatory disease, a disease related to FXII/FXII-induced kinin formation, atrial fibrillation, acute coronary syndromes (ACS), acute limb ischemia, acute respiratory distress syndrome (ARDS; or acute lung injury) and lupus nephritis (including acute lupus nephritis or chronic lupus nephritis).

In one example, the complement-mediated disorder, the neutrophil-mediated disorder and/or the blood coagulation disorder is selected from the group consisting of paroxysmal nocturnal haemoglobinuria (PNH), atypical haemolytic uraemic syndrome (aHUS), thrombocytopenic purpura (TTP), thrombotic microangiopathy, C3 glomerulopathy, membranoproliferative glomerulonephritis (including anti-Thy 1 glomerulonephritis, anti-conA diffuse proliferative glomerulonephritis and/or passive heymann nephritis), and/or, Guillain-Barré syndrome, myasthenia gravis (including autoimmune gyasthenia gravis, demyelinating allergic encephalomyelitis, IgG immune complex alveolitis, reverse passive arthus reaction), systemic lupus erythematosus (SLE), IgA nephropathy, autoimmune haemolytic anemia, pemphigus (including pemphigus vulgaris), pemphigoid (including bullous pemphigoid), anti-phospholipid syndrome, polytrauma, haemodialysis, post-infection HUS, macular degeneration, ANCA-associated vasculitis, atherosclerosis, mood disorders, chronic inflammatory demyelinating polyneuropathy (CIDP), anaphylaxis, cerebral malaria, dermatomyositis, osteoarthritis, dementia, glaucoma, diabetic angiopathy, myocardial infarction, anti-glomerular basement membrane (GBM) nephritis (or Goodpasture's syndrome), autoimmune epilepsy, dermatitis herpetiformis, eosinophilic granulomatosis with polyangiitis (EGPA; or Churg-Strauss syndrome), Sjögren's syndrome and Sjögren's syndrome vasculitis.

In one example, the complement-mediated disorder, the neutrophil-mediated disorder and/or the blood coagulation disorder is an inflammatory joint condition. For example, the inflammatory joint condition is inflammatory arthritis, rheumatoid arthritis or idiopathic arthritis, e.g., juvenile idiopathic arthritis. In one example, the inflammatory arthritis is psoriatic arthritis.

In one example, the complement-mediated disorder, the neutrophil-mediated disorder and/or the blood coagulation disorder is an inflammatory eye condition. For example, the inflammatory eye condition is uveitis.

In one example, the complement-mediated disorder, the neutrophil-mediated disorder and/or the blood coagulation disorder is an inflammatory lung condition. For example, the inflammatory lung condition is a pulmonary disease associated with neutrophil infiltration, such as chronic obstructive pulmonary disease (COPD) and exacerbated forms thereof (such as acute exacerbated COPD (AECOPD) and complications arising therefrom or manifestations thereof such as chronic bronchitis, oxidative stress, emphysema, mucus hypersecretion, arrhythmias, or pulmonale pneumonia and lung cancer).

In one example, the complement-mediated disorder, the neutrophil-mediated disorder and/or the blood coagulation disorder is an inflammatory neurological condition. For example, the inflammatory neurological condition is Devic's disease (neuromyelitis optica), a viral infection in the brain or multiple sclerosis (including chronic progressive multiple sclerosis or relapsing-remitting multiple sclerosis).

In one example, the complement-mediated disorder, the neutrophil-mediated disorder and/or the blood coagulation disorder is an autoimmune intestinal disorder. For example, the autoimmune intestinal disorder is Crohn's disease or ulcerative colitis.

In one example, the complement-mediated disorder, the neutrophil-mediated disorder and/or the blood coagulation disorder is psoriasis.

In one example, the cancer (including angiogenesis thereof) or metastasis thereof.

In one example, the complement-mediated disorder, the neutrophil-mediated disorder and/or the blood coagulation disorder is solid organ transplantation. For example, the solid organ transplantation is lung transplantation. In a further example, the solid organ transplantation is renal transplantation.

In one example, the complement-mediated disorder, the neutrophil-mediated disorder and/or the blood coagulation disorder is ischemia reperfusion injury before, during or after transplantation.

In one example, the complement-mediated disorder, the neutrophil-mediated disorder and/or the blood coagulation disorder is delayed graft function.

In one example, the complement-mediated disorder, the neutrophil-mediated disorder and/or the blood coagulation disorder is asthma and exacerbated forms thereof. For example, the asthma and exacerbated forms thereof is allergic asthma, neutrophilic asthma, mixed granulocytic asthma, severe asthma, moderate asthma, poorly controlled or uncontrolled asthma, refractory asthma or chronic asthma.

In one example, the complement-mediated disorder, the neutrophil-mediated disorder and/or the blood coagulation disorder is neutrophilic dermatosis or a neutrophilic skin lesion. For example, the neutrophilic dermatosis is selected from the group consisting of pustular psoriasis, amicrobial pustulosis of the folds (APF); CARD14-mediated pustular psoriasis (CAMPS); cryopyrin associated periodic syndromes (CAPS); deficiency of interleukin-1 receptor (DIRA); deficiency of interleukin-36 receptor antagonist (DIRTA); hidradenitis suppurativa (HS); palmoplantar pustulosis; pyogenic arthritis; pyoderma gangrenosum and acne (PAPA); pyoderma gangrenosum, acne, and hidradenitis suppurativa (PASH); pyoderma gangrenosum (PG); skin lesions of Behcet's disease; Still's disease; Sweet syndrome; subcorneal pustulosis (Sneddon-Wilkinson); pustular psoriasis; palmoplantar pustulosis; acute generalized exanthematic pustulosis; infantile acropustulosis; synovitis, acne, pustulosis; hyperostosis and osteitis (SAPHO) syndrome; bowel-associated dermatosis-arthritis syndrome (BADAS); neutrophilic dermatosis of the dorsal hands; neutrophilic eccrine hidradenitis; erythema elevatum diutinum; and Pyoderma gangrenosum.

In one example, the complement-mediated disorder, neutrophil-mediated disorder and/or the blood coagulation disorder is an ischemic stroke with reperfusion. For example, the complement-mediated disorder, neutrophil-mediated disorder and/or the blood coagulation disorder is a secondary aspect of stroke (e.g., the secondary aspects of ischemic or hemorrhagic stroke).

In one example, the complement-mediated disorder, neutrophil-mediated disorder and/or the blood coagulation disorder is a neurotraumatic disorder. For example, the neurotraumatic disorder is a traumatic injury of the central nervous system (CNS), including a spinal cord injury and a traumatic brain injury. In one example, the complement-mediated disorder, neutrophil-mediated disorder and/or the blood coagulation disorder is a spinal cord injury. In another example, the complement-mediated disorder, neutrophil-mediated disorder and/or the blood coagulation disorder is a traumatic brain injury.

In one example, the complement-mediated disorder, neutrophil-mediated disorder and/or the blood coagulation disorder is an ischemia-reperfusion injury (IRI). For example, the IRI is caused by a natural event (e.g., restoration of blood flow following a myocardial infarction), a trauma, or by one or more surgical procedures or other therapeutic interventions that restore blood flow to a tissue or organ that had been subjected to a diminished supply of blood. Such surgical procedures can include, for example, coronary artery bypass graft surgery, coronary angioplasty, organ transplant surgery, elective surgery, reconstructive surgery, vascular surgery, cardiac surgery, trauma surgery, crash or crush surgery, cancer surgery, orthopedic surgery, transplantation, or minimally invasive surgery. In one example, the surgical procedure can include the insertion of a device for delivery of a pharmacologically active substance, such as a thrombolytic agent or vasodilator, or a device to mechanically remove complete or partial obstructions, e.g., obstructions of blood vessels.

In one example, the complement-mediated disorder, neutrophil-mediated disorder and/or the blood coagulation disorder is a venous, arterial or capillary thrombus. For example, the venous or arterial thrombus is associated with a disease or condition selected from the group consisting of stroke, myocardial infarction, deep vein thrombosis (DVT), portal vein thrombosis, thromboembolism, renal vein thrombosis, jugular vein thrombosis, cerebral venous sinus thrombosis, Budd-Chiari syndrome, silent brain ischemia (SBI), and Paget-Schroetter disease.

In one example, the complement-mediated disorder, neutrophil-mediated disorder and/or the blood coagulation disorder is a chronic and/or an acute thromboembolism. For example, the chronic and/or acute thromboembolism is a pulmonary embolism, cerebral thromboembolism following atrial fibrillation-induced thrombus formation (e.g., stroke prevention in atrial fibrillation (SPAF)).

In one example, the stroke is thrombic stroke. In another example, the stroke is stroke prevention in atrial fibrillation (SPAF).

In one example, the thromboembolism is a pulmonary embolism. In another example, the thromboembolism is a systemic embolism. In a further example, the thromboembolism is chronic thromboembolic pulmonary hypertension.

In one example, the complement-mediated disorder, neutrophil-mediated disorder and/or the blood coagulation disorder is contact-mediated thrombo-inflammation.

In one example, the complement-mediated disorder, neutrophil-mediated disorder and/or the blood coagulation disorder is atrial fibrillation.

In one example, the complement-mediated disorder, neutrophil-mediated disorder and/or the blood coagulation disorder is an acute coronary syndrome (ACS).

In one example, the complement-mediated disorder, neutrophil-mediated disorder and/or the blood coagulation disorder is acute limb ischemia.

In one example, the complement-mediated disorder, neutrophil-mediated disorder and/or the blood coagulation disorder is thrombus formation during and/or after contacting blood of a human or animal subject with artificial surfaces. For example, in subjects with valve replacements, stents, percutaneous coronary intervention (PCI), extracorporeal membrane oxygenation (ECMO), or undergoing cardiopulmonary bypass surgery (CPB surgery).

In one example, the complement-mediated disorder, neutrophil-mediated disorder and/or the blood coagulation disorder is interstitial lung disease. For example, the interstitial lung disease is fibroproliferative and/or idiopathic pulmonary fibrosis.

In one example, the complement-mediated disorder, neutrophil-mediated disorder and/or the blood coagulation disorder is inflammation. For example, the inflammation is a neurological inflammatory disease (or neuroinflammatory disease). In one example, the neurological inflammatory disease is spinal cord injury (SCI), stroke, traumatic brain injury (TBI), secondary brain edema, edema of the central nervous system, multiple sclerosis (MS), transverse myelitis, or neuromyelitis optica (Devic's disease).

In one example, the complement-mediated disorder, neutrophil-mediated disorder and/or the blood coagulation disorder is fibrinolysis.

In one example, the complement-mediated disorder, neutrophil-mediated disorder and/or the blood coagulation disorder is angiogenesis.

In one example, the complement-mediated disorder, neutrophil-mediated disorder and/or the blood coagulation disorder is a thrombo-inflammatory disease.

In one example, the complement-mediated disorder, neutrophil-mediated disorder and/or the blood coagulation disorder is a disease related to FXII/FXII-induced kinin formation. For example, the disease is selected from the group consisting of hereditary angioedema, bacterial infections of the lung, trypanosoma infections, hypotensive shock, pancreatitis, chagas disease, articular gout, arthritis, disseminated intravascular coagulation (DIC) and sepsis.

In one example, the complement-mediated disorder, neutrophil-mediated disorder and/or the blood coagulation disorder is lupus nephritis. For example, the lupus nephritis is acute lupus nephritis or chronic lupus nephritis.

In one example, the complement-mediated disorder, neutrophil-mediated disorder and/or the blood coagulation disorder is systemic lupus erythematosus (SLE).

In one example, the complement-mediated disorder, the neutrophil-mediated disorder and/or the blood coagulation disorder is acute respiratory distress syndrome (ARDS; or acute lung injury). For example, the ARDS is mild, moderate or severe 35 ARDS.

In one example, the complement-mediated disorder, the neutrophil-mediated disorder and/or the blood coagulation disorder is paroxysmal nocturnal haemoglobinuria (PNH).

In one example, the complement-mediated disorder, the neutrophil-mediated disorder and/or the blood coagulation disorder is atypical haemolytic uraemic syndrome (aHUS).

In one example, the complement-mediated disorder, the neutrophil-mediated disorder and/or the blood coagulation disorder is thrombocytopenic purpura (TTP).

In one example, the complement-mediated disorder, the neutrophil-mediated disorder and/or the blood coagulation disorder is thrombotic microangiopathy.

In one example, the complement-mediated disorder, the neutrophil-mediated disorder and/or the blood coagulation disorder is C3 glomerulopathy. For example, the C3 glomerulopathy is membranoproliferative glomerulonephritis (including anti-Thy 1 glomerulonephritis, anti-conA diffuse proliferative glomerulonephritis and/or passive heymann nephritis).

In one example, the complement-mediated disorder, the neutrophil-mediated disorder and/or the blood coagulation disorder is Guillain-Barré syndrome.

In one example, the complement-mediated disorder, the neutrophil-mediated disorder and/or the blood coagulation disorder is myasthenia gravis. For example, the myasthenia gravis is autoimmune gyasthenia gravis, demyelinating allergic encephalomyelitis, IgG immune complex alveolitis or reverse passive arthus reaction.

In one example, the complement-mediated disorder, the neutrophil-mediated disorder and/or the blood coagulation disorder is IgA nephropathy.

In one example, the complement-mediated disorder, the neutrophil-mediated disorder and/or the blood coagulation disorder is autoimmune haemolytic anemia.

In one example, the complement-mediated disorder, the neutrophil-mediated disorder and/or the blood coagulation disorder is pemphigus. For example, the pemphigus is pemphigus vulgaris.

In one example, the complement-mediated disorder, the neutrophil-mediated disorder and/or the blood coagulation disorder is pemphigoid. For example, the pemphigoid is bullous pemphigoid.

In one example, the complement-mediated disorder, the neutrophil-mediated disorder and/or the blood coagulation disorder is an anti-phospholipid syndrome.

In one example, the complement-mediated disorder, the neutrophil-mediated disorder and/or the blood coagulation disorder is polytrauma.

In one example, the complement-mediated disorder, the neutrophil-mediated disorder and/or the blood coagulation disorder is haemodialysis.

In one example, the complement-mediated disorder, the neutrophil-mediated disorder and/or the blood coagulation disorder is post-infection haemolytic-uremic syndrome (HUS).

In one example, the complement-mediated disorder, the neutrophil-mediated disorder and/or the blood coagulation disorder is macular degeneration.

In one example, the complement-mediated disorder, the neutrophil-mediated disorder and/or the blood coagulation disorder is ANCA-associated vasculitis.

In one example, the complement-mediated disorder, the neutrophil-mediated disorder and/or the blood coagulation disorder is atherosclerosis.

In one example, the complement-mediated disorder, the neutrophil-mediated disorder and/or the blood coagulation disorder is a mood disorders.

In one example, the complement-mediated disorder, the neutrophil-mediated disorder and/or the blood coagulation disorder is chronic inflammatory demyelinating polyneuropathy (CIDP).

In one example, the complement-mediated disorder, the neutrophil-mediated disorder and/or the blood coagulation disorder is anaphylaxis.

In one example, the complement-mediated disorder, the neutrophil-mediated disorder and/or the blood coagulation disorder is cerebral malaria.

In one example, the complement-mediated disorder, the neutrophil-mediated disorder and/or the blood coagulation disorder is dermatomyositis.

In one example, the complement-mediated disorder, the neutrophil-mediated disorder and/or the blood coagulation disorder is osteoarthritis.

In one example, the complement-mediated disorder, the neutrophil-mediated disorder and/or the blood coagulation disorder is dementia.

In one example, the complement-mediated disorder, the neutrophil-mediated disorder and/or the blood coagulation disorder is glaucoma.

In one example, the complement-mediated disorder, the neutrophil-mediated disorder and/or the blood coagulation disorder is diabetic angiopathy.

In one example, the complement-mediated disorder, the neutrophil-mediated disorder and/or the blood coagulation disorder is myocardial infarction.

In one example, the complement-mediated disorder, the neutrophil-mediated disorder and/or the blood coagulation disorder is anti-glomerular basement membrane (GBM) nephritis (or Goodpasture's syndrome).

In one example, the complement-mediated disorder, the neutrophil-mediated disorder and/or the blood coagulation disorder is autoimmune epilepsy.

In one example, the complement-mediated disorder, the neutrophil-mediated disorder and/or the blood coagulation disorder is dermatitis herpetiformis.

In one example, the complement-mediated disorder, the neutrophil-mediated disorder and/or the blood coagulation disorder is eosinophilic granulomatosis with polyangiitis (EGPA; or Churg-Strauss syndrome).

In one example, the complement-mediated disorder, the neutrophil-mediated disorder and/or the blood coagulation disorder is Sjögren's syndrome. For example, the disorder is Sjögren's syndrome vasculitis.

In one example, the subject has a condition requiring prophylactic treatment.

In one example, the sCR1 conjugate or composition comprising the sCR1 conjugate of the present disclosure is administered to the subject in an amount to reduce the severity of the complement-mediated disorder, the neutrophil-mediated disorder and/or the blood coagulation disorder in the subject.

In one example, the sCR1 conjugate or composition comprising the sCR1 conjugate of the present disclosure is administered to the subject in an amount sufficient to reduce the number of neutrophils in a subject without inducing neutropenia.

In one example of any method described herein, the subject is a mammal, for example a primate such as a human.

Methods of treatment described herein can additionally comprise administering a further compound to reduce, treat or prevent the effect of the complement-mediated disorder, the neutrophil-mediated disorder and/or the blood coagulation disorder.

The present disclosure provides a kit comprising at least one sCR1 conjugate or composition comprising a sCR1 conjugate of the disclosure packaged with instructions for use in inhibiting complement activity and/or G-CSF activity in a subject. Optionally, the kit additionally comprises a further therapeutically active compound or drug.

The present disclosure also provides a kit comprising at least one sCR1 conjugate or composition comprising a sCR1 conjugate of the disclosure packaged with instructions for use in inhibiting complement activity and/or antagonising activity of Factor XII/XIIa and/or antagonising activation of Factor XII/XIIa in a subject. Optionally, the kit additionally comprises a further therapeutically active compound or drug.

The present disclosure also provides a kit comprising at least one sCR1 conjugate or composition comprising a sCR1 conjugate of the disclosure packaged with instructions for use in inhibiting complement activity and/or antagonising activity of Factor XI/XIa and/or antagonising activation of Factor XI/XIa in a subject. Optionally, the kit additionally comprises a further therapeutically active compound or drug.

The present disclosure further provides a kit comprising at least one sCR1 conjugate or composition comprising a sCR1 conjugate of the disclosure packaged with 5 instructions for use in treating or preventing a complement-mediated disorder, a neutrophil-mediated disorder and/or a blood coagulation disorder in a subject. Optionally, the kit additionally comprises a further therapeutically active compound or drug.

The present disclosure also provides a kit comprising at least one sCR1 conjugate or composition comprising a sCR1 conjugate of the disclosure packaged with instructions to administer the conjugate or composition to a subject who is suffering from or at risk of suffering from a complement-mediated disorder, a neutrophil-mediated disorder and/or a blood coagulation disorder, optionally, in combination with a further therapeutically active compound or drug.

Exemplary effects of sCR1 conjugates or compositions of the present disclosure are described herein and are to be taken to apply mutatis mutandis to the examples of the disclosure set out in the previous five paragraphs.

### BRIEF DESCRIPTION OF FIGURES

FIG. 1 is a graphical representation showing the effect of sialylation of sCR1(1392)-8His on plasma half-life.

KEY TO SEQUENCE LISTING

| | |
|---|---|
| SEQ ID NO: 1 | amino acid sequence of soluble complement receptor 1 (sCR1) with |
| | the N-terminal endogenous human CR1 signal peptide |
| SEQ ID NO: 2 | amino acid sequence of mature soluble complement receptor 1 |
| | (sCR1(1971)) lacking the N-terminal endogenous human CR1 |
| | signal peptide |
| SEQ ID NO: 3 | amino acid sequence of truncated mature soluble complement |
| | receptor 1 (sCR1(1392)) lacking the N-terminal endogenous human |
| | CR1 signal peptide |
| SEQ ID NO: 4 | amino acid sequence of truncated mature soluble complement |
| | receptor 1 (sCR1(939)) lacking the N-terminal endogenous human |
| | CR1 signal peptide |
| SEQ ID NO: 5 | amino acid sequence of truncated mature soluble complement |
| | receptor 1 (sCR1(490-1392)) |
| SEQ ID NO: 6 | amino acid sequence of truncated mature soluble complement |
| | receptor 1 (sCR1(490-1971)) |
| SEQ ID NO: 7 | amino acid sequence of truncated mature soluble complement |
| | receptor 1 (sCR1(234)) lacking the N-terminal endogenous human |
| | CR1 signal peptide |
| SEQ ID NO: 8 | amino acid sequence of truncated mature soluble complement |
| | receptor 1 (sCR1(489)) lacking the N-terminal endogenous human |
| | CR1 signal peptide |
| SEQ ID NO: 9 | amino acid sequence of truncated mature soluble complement |
| | receptor 1 (sCR1(940-1971)) |
| SEQ ID NO: 10 | amino acid sequence of truncated mature soluble complement |
| | receptor 1 (sCR1(490-939)) |
| SEQ ID NO: 11 | amino acid sequence of truncated mature soluble complement |
| | receptor 1 (sCR1(940-1392)) |
| SEQ ID NO: 12 | amino acid sequence of truncated mature soluble complement |
| | receptor 1 (sCR1(1393-1971)) |
| SEQ ID NO: 13 | amino acid sequence of sCR1 LHR-A |
| SEQ ID NO: 14 | amino acid sequence of sCR1 LHR-B |
| SEQ ID NO: 15 | amino acid sequence of sCR1 LHR-C |
| SEQ ID NO: 16 | amino acid sequence of sCR1 LHR-D |
| SEQ ID NO: 17 | 8xHis tag |
| SEQ ID NO: 18 | amino acid sequence of endogenous signal peptide |
| SEQ ID NO: 19 | amino acid sequence of exogenous signal peptide |
| SEQ ID NO: 20 | amino acid sequence of His tagged soluble complement receptor 1 |
| | (sCR1(1971)-8His) with N-terminal endogenous signal peptide |
| SEQ ID NO: 21 | amino acid sequence of His tagged truncated soluble complement |
| | receptor 1 (sCR1(1392)-8His) with N-terminal endogenous signal |
| | peptide |
| SEQ ID NO: 22 | amino acid sequence of truncated mature soluble complement |
| | receptor 1 (sCR1(939)-8His) with N-terminal endogenous signal |
| | peptide |
| SEQ ID NO: 23 | amino acid sequence of His tagged truncated soluble complement |
| | receptor 1 (sCR1(490-1392)-8His) with N-terminal exogenous |
| | signal peptide |
| SEQ ID NO: 24 | amino acid sequence of His tagged truncated soluble complement |
| | receptor 1 (sCR1(490-1971)-8His) with N-terminal exogenous |
| | signal peptide |
| SEQ ID NO: 25 | amino acid sequence of His tagged truncated soluble complement |
| | receptor 1 (sCR1(234)-8His) with N-terminal endogenous signal |
| | peptide |
| SEQ ID NO: 26 | amino acid sequence of His tagged truncated soluble complement |
| | receptor 1 (sCR1(489)-8His) with N-terminal endogenous signal |
| | peptide |
| SEQ ID NO: 27 | amino acid sequence of His tagged truncated soluble complement |
| | receptor 1 (sCR1(940-1971)-8His) with N-terminal exogenous |
| | signal peptide |
| SEQ ID NO: 28 | amino acid sequence of His tagged truncated soluble complement |
| | receptor 1 (sCR1(490-939)-8His) with N-terminal exogenous signal |
| | peptide |
| SEQ ID NO: 29 | amino acid sequence of His tagged truncated soluble complement |
| | receptor 1 (sCR1(940-1392)-8His) with N-terminal exogenous |
| | signal peptide |
| SEQ ID NO: 30 | amino acid sequence of His tagged truncated soluble complement |
| | receptor 1 (sCR1(1393-1971)-8His) with N-terminal exogenous |
| | signal peptide |
| SEQ ID NO: 31 | GS13 peptide linker |
| SEQ ID NO: 32 | GS15 peptide linker |
| SEQ ID NO: 33 | GS16 peptide linker |
| SEQ ID NO: 34 | GS20 peptide linker |
| SEQ ID NO: 35 | GS30 peptide linker |
| SEQ ID NO: 36 | amino acid sequence of C1.2 VH |
| SEQ ID NO: 37 | amino acid sequence of C1.2 VL |
| SEQ ID NO: 38 | amino acid sequence of C1.2G VH |
| SEQ ID NO: 39 | amino acid sequence of C1.2G VL |
| SEQ ID NO: 40 | amino acid sequence of C1.2 VH CDR1 |
| SEQ ID NO: 41 | amino acid sequence of C1.2 VH CDR2 |
| SEQ ID NO: 42 | amino acid sequence of C1.2 VH CDR3 |
| SEQ ID NO: 43 | amino acid sequence of C1.2 VL CDR1 |
| SEQ ID NO: 44 | amino acid sequence of C1.2 VL CDR2 |
| SEQ ID NO: 45 | amino acid sequence of C1.2 VL CDR3 |
| SEQ ID NO: 46 | amino acid sequence of 5E2VR81 VH |
| SEQ ID NO: 47 | amino acid sequence of 5E2VR81 VL |
| SEQ ID NO: 48 | amino acid sequence 25-335 of Homo sapiens G-CSFR (hG-CSFR) |
| | with a C-terminal polyhistidine tag |
| SEQ ID NO: 49 | amino acid sequence of truncated soluble complement receptor 1 |
| | conjugated to C1.2scFv (sCR1(1392)-GS16-C1.2scFvLH) with N- |
| | terminal endogenous signal peptide |
| SEQ ID NO: 50 | amino acid sequence of truncated soluble complement receptor 1 |
| | conjugated to C1.2scFv (sCR1(1392)-GS16-C1.2scFvHL) with N- |
| | terminal endogenous signal peptide |
| SEQ ID NO: 51 | amino acid sequence of truncated soluble complement receptor 1 |
| | conjugated to 5E2VR81scFv (sCR1(1392)-GS16- |
| | 5E2VR81scFvLH) with N-terminal endogenous signal peptide |
| SEQ ID NO: 52 | amino acid sequence of truncated soluble complement receptor 1 |
| | conjugated to 5E2VR81scFv (sCR1(1392)-GS16- |
| | 5E2VR81scFvHL) with N-terminal endogenous signal peptide |
| SEQ ID NO: 53 | amino acid sequence of truncated soluble complement receptor 1 |
| | conjugated to C1.2GscFv (sCR1(1392)-GS16-C1.2GscFvLH) with |
| | N-terminal endogenous signal peptide |
| SEQ ID NO: 54 | amino acid sequence of truncated soluble complement receptor 1 |
| | conjugated to C1.2GscFv (sCR1(1392)-GS16-C1.2GscFvHL) with |
| | N-terminal endogenous signal peptide |
| SEQ ID NO: 55 | GS16 peptide linker |
| SEQ ID NO: 56 | amino acid sequence of 3F7 VH |
| SEQ ID NO: 57 | amino acid sequence of 3F7 VL |
| SEQ ID NO: 58 | amino acid sequence of 3F7G VH |
| SEQ ID NO: 59 | amino acid sequence of 3F7G VL |
| SEQ ID NO: 60 | amino acid sequence of affinity matured 3F7 VH |
| SEQ ID NO: 61 | amino acid sequence of affinity matured 3F7 VL |
| SEQ ID NO: 62 | amino acid sequence of truncated soluble complement receptor 1 |
| | conjugated to 3F7scFv (sCR1(1392)-GS16-3F7scFvHL) with N- |
| | terminal endogenous signal peptide |
| SEQ ID NO: 63 | amino acid sequence of truncated soluble complement receptor 1 |
| | conjugated to 3F7GscFv (sCR1(1392)-GS16-3F7GscFvHL) with |
| | N-terminal endogenous signal peptide |
| SEQ ID NO: 64 | amino acid sequence of truncated soluble complement receptor 1 |
| | conjugated to 3F7^{aff}scFv (sCR1(392)-GS16-3F7^{aff}scFvHL) with |
| | N-terminal endogenous signal peptide |
| SEQ ID NO: 65 | amino acid sequence from a human Factor XII |

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J. Mol. Biol. 242, 309-15 320, 1994, Chothia and Lesk J. Mol Biol. 196:901 -917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

Complement receptor type 1 (CR1), also known as C3b/C4b receptor or CD35 is a member of the family of regulators of complement activation. CR1 is present on the membranes of erythrocytes, monocytes/macrophages, granulocytes, B cells, some T cells, splenic follicular dendritic cells, and glomerular podocytes, and mediates cellular binding to particles and immune complexes that have activated complement. The encoded protein has a 41 amino acid signal peptide, an extracellular domain of 1930 residues, a 25 residue transmembrane domain and a 43 amino acid C-terminal cytoplasmic region. For the purposes of nomenclature only and not limitation an exemplary sequence of human CR1 is set out in GenBank Accession no. NP_000564.

Soluble complement receptor type 1 (sCR1) is naturally produced by cleavage of cell surface CR1 and plays a role in the control of complement activation at sites of inflammation. It should be understood that reference to “sCR1” refers to truncated CR1, which lacks the trans-membrane and cytoplasmic domains. For the purposes of nomenclature only and not limitation an exemplary sequence of human sCR1 is set out in SEQ ID NO: 1. Positions of amino acids are referred to herein by reference to sCR1 protein consisting of 1971 amino acids (e.g., as set out in SEQ ID NO: 1). Full length sCR1 comprises four long homologous repeat (LHR) regions, i.e., LHR-A, B, C and D. LHR regions may be defined with reference to human sCR1 (as set forth in SEQ ID NO: 1). For example, LHR-A comprises amino acids 42 to 489 of SEQ ID NO: 1, LHR-B comprises amino acids 490 to 939 of SEQ ID NO: 1, LHR-C comprises amino acids 940 to 1392 of SEQ ID NO: 1 and LHR-D comprises amino acids 1393 to 1971 of SEQ ID NO: 1. Each LHR comprises short consensus repeat (SCR) sequences with a total of 30 SCR sequences, each having 60 to 70 amino acids. For example, LHR-A comprises SCRs 1 to 7 (corresponding to amino acids 42 to 489 of SEQ ID NO: 1), LHR-B comprises SCRs 8 to 14 (corresponding to amino acids 491 to 939 of SEQ ID NO: 1), LHR-C comprises SCRs 15 to 21 (corresponding to amino acids 941 to 1389 of SEQ ID NO: 1), and LHR-D comprises SCRs 22 to 28 (corresponding to amino acids 1394 to 1842 of SEQ ID NO: 1) and SCRs 29 to 30 (corresponding to amino acids 1846 to 1967 of SEQ ID NO: 1). A sequence of mature human sCR1 lacks the N-terminal signal peptide corresponding to amino acids 1 to 41 of SEQ ID NO: 1. For example, a sequence of mature human sCR1 (i.e., lacking the N-terminal signal peptide) is set forth in SEQ ID NO: 2.

The sequence of sCR1 from other species can be determined using sequences provided herein and/or in publicly available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)).

As used herein the phrase “corresponding to” in reference to the position of an amino acid in SEQ ID NO: 1 should be understood as reference to an amino acid residue or position within a sCR1 sequence, and not necessarily a sequence comprising SEQ ID 35 NO: 1. For example, reference to “a position corresponding to amino acids 42 to 939 of SEQ ID NO: 1” in a sCR1 sequence comprising a 41 amino acid N-terminal truncation (i.e., mature sCR1) would necessarily refer to amino acids at position 1 to 898. In one example, the sCR1 comprises a sequence set forth in SEQ ID NO: 1.

As used herein, the term “variant” refers to a sCR1 which has undergone deletion or truncation of one or more amino acids using well known techniques.

As used herein, the term “inhibit(s)” or “inhibiting” in the context of complement activity shall be understood to mean that the sCR1 variant conjugate of the present disclosure reduces or decrease the level of complement activity. It will be apparent from the foregoing that the sCR1 variant conjugate of the present disclosure need not completely inhibit complement activity, rather it need only reduce activity by a statistically significant amount, for example, by at least about 10%, or about 20%, or about 30%, or about 40%, or about 50%, or about 60%, or about 70%, or about 80%, or about 90%, or about 95%. Methods for determining inhibition of complement activity are known in the art and/or described herein.

As used herein, the term “serum half-life” or “plasma half-life” in the context of the present disclosure refers to the period of time required for the concentration or amount of the sCR1 conjugate in the serum to be reduced by 50% (i.e., one half) for example due to degradation and/or clearance or sequestration by natural mechanisms. The skilled person would recognise that the serum half-life of sCR1 in a subject is dependent on various physiological conditions (e.g., health status, body size/weight). In a healthy human subject, the serum half-life of sCR1 is approximately 70 hours (3 days).

Methods for determining the serum half-life of sCR1 are known in the art and include, for example, pharmacokinetic analysis. For the purposes of the present disclosure, an “increase” or “enhanced” serum half-life refers to an elevation or increase in time taken for the serum concentration of the sCR1 variant to be reduced by 50%, compared to a sCR1 set forth in SEQ ID NO: 2.

G-CSF is a major regulator of granulocyte production. G-CSF is produced by bone marrow stromal cells, endothelial cells, macrophages, and fibroblasts, and production is induced by inflammatory stimuli. G-CSF acts through the G-CSF receptor (G-CSFR), which is expressed on early myeloid progenitors, mature neutrophils, monocytes/macrophages, T and B lymphocytes and endothelial cells.

Reference herein to “granulocyte colony-stimulating factor” (G-CSF) includes native forms of G-CSF, mutant forms thereof, e.g., filgrastim and pegylated forms of G-CSF or filgrastim. This term also encompasses mutant forms of G-CSF retaining activity to bind to G-CSFR (e.g., human G-CSFR) and induce signaling.

For the purposes of nomenclature only and not limitation, an exemplary sequence of a human G-CSFR is set out in NCBI Reference Sequence: NP_000751.1 (and set out in SEQ ID NO: 48). The sequence of G-CSFR from other species can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)). Reference to human G-CSFR may be abbreviated to hG-CSFR and, for example, reference to cynomolgus monkey G-CSFR may be abbreviated to cynoG-CSFR. Reference to soluble G-CSFR refers to polypeptides comprising the ligand binding region of G-CSFR. The Ig and CRH domains of the G-CSFR are involved in ligand binding and receptor dimerization (Layton et al., J. Biol Chem., 272: 29735-29741, 1997 and Fukunaga et al, EMBO J. 10: 2855-2865, 1991). Soluble forms of G-CSFR comprising these portions of the receptor have been used in various studies of the receptor and mutation of the free cysteines at positions 78, 163, and 228 of the receptor assists in expression and isolation of the soluble receptor polypeptide (Mine et al., Biochem., 43: 2458-2464 2004) without affecting ligand binding.

As used herein, the term “coagulation factor” refers to a factor that is associated with the formation of a blot clot, i.e., blood coagulation. Coagulation factors are known in the art and include without limitation factor I, factor II, factor III, factor V, factor VII, factor VIII, factor IX, factor X, factor XI, factor XII and factor XIII or an activated form of any of the foregoing. This term also includes recombinant forms of coagulation factors and/or modified forms thereof, e.g., as is known in the art and/or described herein.

Coagulation Factor XII, also known as Hageman factor or FXII, is a plasma protein. It is the zymogen form of Factor XIIa, an enzyme of the serine protease (or serine endopeptidase) class. In humans, Factor XII is encoded by the F12 gene. For the purposes of nomenclature only and not limitation exemplary sequences of human Factor XII is set out in NCBI Reference Sequence: NP_000496.2; in NCPI protein accession number NP_000496 and in SEQ ID NO: 65. Additional sequences of Factor XII can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)).

As used herein, the term “Factor XII inhibitor” or “FXII inhibitor” or “inhibitor of FXII” refers to an inhibitor of either or both of Factor XII (prior to activation, i.e., its zymogen) and activated Factor XII (FXIIa) as well as to the activation of FXII. Thus, “inhibitor(s) of FXII” can include inhibitors of either or both of FXII and FXIIa (also termed αFXIIa) as well as the activation of FXII, including the FXIIa cleavage products FXIIa alpha and FXIIa beta (also termed FXIIf). FXII inhibitors encompass functional variants and fragments of the wild-type inhibitor. A functional variant or fragment is a molecule that retains at least 50% (e.g., about 50%, or about 60%, or about 70%, or about 80%, or about 90%, or about 95%, or about 99%, or about 100%) of the ability of the wild-type molecule to inhibit FXII, FXIIa or the activation of FXII. In one example, the FXII inhibitors are non-endogenous inhibitors; that is, they are not inhibitors that occur naturally in the human or animal body.

The term “amidolytic activity” refers to the ability of the protein of the present disclosure to catalyse the hydrolysis of at least one peptide bond in another polypeptide.

As used herein, the term “conjugated” shall be understood to encompass chemical conjugation, which can be non-covalent or covalent or genetic conjugation (also referred to as “fusion”). In one example, the conjugation is covalent, e.g., a disulphide bond. In one example, a conjugate of the disclosure is a fusion protein comprising two components linked by genetic conjugation.

The term “recombinant” shall be understood to mean the product of artificial genetic recombination. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is cap/able of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

An “anti-FXII antibody” includes antibodies that bind to and/or inhibit either or both of the zymogen of FXII and the activated protein (FXIIa), including the FXIIa alpha and FXIIa beta cleavage fragments. In some examples, the antibody binds specifically to FXIIa or the alpha or beta chain fragments of FXIIa.

As used herein the term “germlined” antibody refers to an antibody where some or all somatic mutations that introduced changes into the framework residues are reversed to the original sequence present in the genome, e.g., a human genome. In this regard, not all changes need to be reversed in a germlined antibody.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plükthun J. Mol. Biol., 309: 657-670, 2001. For example, according to the numbering system of Kabat, V_{H} framework regions (FRs) and CDRs are positioned as follows: residues 1-30 (FR1), 31-(CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (1-R4). The present disclosure is not limited to FRs and CDRs as defined by the Kabat numbering system, but includes all numbering systems, including those discussed above. In one example, reference herein to a CDR (or a FR) is in respect of those regions according to the Kabat numbering system.

“Framework regions” (FRs) are those variable region residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site (or antigen binding domain) can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a compound or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that a compound of the disclosure reacts or associates more frequently, more rapidly, with greater duration and/or with greater affinity with a particular antigen or cell expressing same than it does with alternative antigens or cells. For example, a conjugate comprising an antibody Fc that binds to G-CSFR (e.g., hG-CSFR) with materially greater affinity (e.g., 20 fold or 40 fold or 60 fold or 80 fold to 100 fold or 150 fold or 200 fold) than it does to other cytokine receptor or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

As used herein, the term “epitope” (syn. “antigenic determinant”) shall be understood to mean a region of hG-CSFR to which a protein comprising an antigen binding site of an antibody binds. This term is not necessarily limited to the specific residues or structure to which the protein makes contact. For example, this term includes the region spanning amino acids contacted by the protein and/or 5-10 or 2-5 or 1-3 amino acids outside of this region. In some examples, the epitope comprises a series of discontinuous amino acids that are positioned close to one another when hG-CSFR is folded, i.e., a “conformational epitope”. For example, a conformational epitope comprises amino acids in one or more or two or more or all of the regions corresponding to 111-115, 170-176, 218-234 and/or 286-300 of SEQ ID NO: 48. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

The term “competitively inhibits” shall be understood to mean that a protein of the disclosure (or an antigen binding site thereof) reduces or prevents binding of a recited antibody or protein to G-CSFR, e.g., to hG-CSFR or Factor XII and/or Factor XIIa. This may be due to the protein (or antigen binding site) and antibody binding to the same or an overlapping epitope. It will be apparent from the foregoing that the protein need not completely inhibit binding of the antibody, rather it need only reduce binding by a 10 statistically significant amount, for example, by at least about 10% or 20% or 30% or 40% or 50% or 60% or 70% or 80% or 90% or 95%. Preferably, the protein reduces binding of the antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to G-CSFR or Factor XII either in the presence or absence of the protein. If less antibody binds in the presence of the protein than in the absence of the protein, the protein is considered to competitively inhibit binding of the antibody. In one example, the competitive inhibition is not due to steric hindrance.

“Overlapping” in the context of two epitopes shall be taken to mean that two epitopes share a sufficient number of amino acid residues to permit a protein (or antigen binding site thereof) that binds to one epitope to competitively inhibit the binding of a protein (or antigen binding site) that binds to the other epitope. For example, the “overlapping” epitopes share at least 1 or 2 or 3 or 4 or 5 or 6 or 7 or 8 or 9 or 20 amino acids.

As used herein, the term “neutralize” shall be taken to mean that a compound is capable of blocking, reducing or preventing G-CSF-mediated signaling in a cell through the G-CSFR or Factor XII/XIIa-mediated activity. Methods for determining neutralization are known in the art and/or described herein.

As used here, the term “antagonise” shall be understood to mean that a protein is capable of blocking, reducing or preventing blood coagulation factor activation and/or activity. Methods for determining antagonising are known in the art and/or described herein.

The phrase “conservative amino acid substitution” refers to replacement or substitution of an amino acid residue with an amino acid residue having a similar side chain and/or hydropathicity and/or hydrophilicity. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

As used herein, the term “condition” refers to a disruption of or interference with normal function, and is not to be limited to any specific condition, and will include diseases or disorders.

“Complement-mediated” disorder, “neutrophil-mediated” and/or “blood coagulation” disorder means that the disorder is mediated, at least in part, by complement, neutrophils and/or blood coagulation factors, respectively.

As used herein, a subject “at risk” of developing a disease or condition or relapse thereof or relapsing may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering a conjugate and/or composition described herein to thereby reduce or eliminate at least one symptom of a specified disease or condition or to slow progression of the disease or condition.

As used herein, the term “preventing”, “prevent” or “prevention” includes providing prophylaxis with respect to occurrence or recurrence of a specified disease or condition in an individual. An individual may be predisposed to or at risk of developing the disease or disease relapse but has not yet been diagnosed with the disease or the relapse.

As used herein, the term “subject” shall be taken to mean any animal including humans, for example a mammal Exemplary subjects include but are not limited to humans and non-human primates. For example, the subject is a human.

### Soluble Complement Receptor Type 1 Variant Conjugates

The present disclosure provides a sCR1 conjugate for use in any method described herein.

sCR1 variants

In one example, the present disclosure provides a sCR1 conjugate comprising an sCR1 variant, wherein the sCR1 variant has improved or increased complement inhibitory activity compared to a conjugate comprising a sequence set forth in SEQ ID NO: 2. The inventors have determined that a sCR1 variant comprising residues 42 to 939 and/or residues 490 to 1392 of SEQ ID NO: 1 have improved and/or increased complement inhibitory activity.

The present disclosure provides a soluble complement receptor type 1 (sCR1) variant conjugate comprising:
(i) an sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising an antigen binding domain that binds to a target and inhibits signaling by or via the target.

The present disclosure also provides a soluble complement receptor type 1 (sCR1) variant conjugate comprising:
(i) an sCR1 variant comprising an amino acid sequence selected from the group consisting of:
   a) an amino acid sequence corresponding to amino acids 42 to 939 of SEQ ID NO: 1;
   b) an amino acid sequence corresponding to amino acids 490 to 1392 of SEQ ID NO: 1; and
(ii) a protein comprising an antigen binding domain that binds to a blood coagulation factor.

For example, the inventors have identified amino acid residues in a sequence set forth in SEQ ID NO: 1 that can be deleted without loss of function or that result in improved function. In one example, the sCR1 variant comprises deletion of between 489 and 1073 amino acids compared to a sequence set forth in SEQ ID NO: 1. For example, the sCR1 variant comprises deletion of 489 or 620 or 1068 or 1073 amino acids compared to a sequence set forth in SEQ ID NO: 1.

In one example, the present disclosure provides a truncated sCR1 comprising between 898 and 1482 amino acids compared to a sequence set forth in SEQ ID NO: 1. For example, the truncated sCR1 comprises 898 or 903 or 1351 or 1482 amino acids compared to a sequence set forth in SEQ ID NO: 1.

In one example, the sCR1 variant of the present disclosure comprises a variant of a sequence set forth in SEQ ID NO: 1, wherein the variant sequence comprises an amino acid sequence corresponding to amino acids 42 to 1392 of SEQ ID NO: 1.

In one example, the sCR1 variant of the present disclosure comprises a variant of a sequence set forth in SEQ ID NO: 1, wherein the variant sequence comprises an amino acid sequence corresponding to amino acids 42 to 939 of SEQ ID NO: 1.

In one example, the sCR1 variant of the present disclosure comprises a variant of a sequence set forth in SEQ ID NO: 1, wherein the variant sequence comprises an amino acid sequence corresponding to amino acids 490 to 1392 of SEQ ID NO: 1.

In one example, the sCR1 variant of the present disclosure comprises a variant of a sequence set forth in SEQ ID NO: 1, wherein the variant sequence comprises an amino acid sequence corresponding to amino acids 490 to 1971 of SEQ ID NO: 1.

In one example, the sCR1 variant of the present disclosure does not comprise or consist of a sequence set forth in SEQ ID NO: 1 and/or SEQ ID NO: 2.

In one example, the sCR1 variant of the present disclosure does not comprise an amino acid sequence corresponding to amino acids 1 to 41 of SEQ ID NO: 1.

In one example, the sCR1 variant of the present disclosure does not comprise an amino acid sequence corresponding to amino acids 940 to 1971 of SEQ ID NO: 1.

In one example, the sCR1 variant of the present disclosure does not comprise an amino acid sequence corresponding to amino acids 1393 to 1971 of SEQ ID NO: 1.

In one example, the sCR1 variant of the present disclosure does not comprise an amino acid sequence corresponding to amino acids 1 to 489 of SEQ ID NO: 1.

In one example, the sCR1 variant is monomeric (i.e., one copy of the sCR1 variant).

In one example, the sCR1 variant is dimeric, or dimerized (i.e., two copies of a sCR1 variant are linked in a fusion protein).

In one example, the sCR1 variant is multimeric, or multimerized (i.e., multiple copies of a sCR1 variant are linked in a fusion protein).

Methods for achieving dimerization or multimerization of the sCR1 variant are known in the art and/or described herein and include, for example, direct conjugation between the two or more sCR1 variants or indirect binding (e.g., by virtue of a linker between the two or more sCR1 variants). In one example, the dimerization or multimerization is formed by a chemical conjugation (e.g., by a disulphide bond or cystine knot) or by genetic fusion.

In one example, two or more of the same sCR1 variant are fused (i.e., expressed as a fusion protein).

In one example, two or more different sCR1 variants are fused (i.e., expressed as a fusion protein).

In one example, the dimerized or multimerized sCR1 variant comprises a linker between the sCR1 variants.

In one example, the disclosure provides a multimeric protein comprising two or more sCR1 variants comprising a multimerization domain, wherein the multimerization domains interact to form the multimeric protein.

In one example, each sCR1 variant in the multimeric protein comprises one sCR1 variant. In another example, one or more sCR1 variants in the multimeric protein comprises two or more sCR1 variants, e.g., the variants are linked in a fusion protein.

In one example, the multimerization domain comprises an immunoglobulin hinge domain

In one example, the multimerization domain is a leucine zipper domain, a cystine knot or an antibody Fc region. For example, the multimerization domain is a leucine zipper domain Suitable leucine zipper polypeptides will be known in the art and include c-Jun and c-Fos leucine zipper domains. Leucine zipper fusions are described in Riley et al., Protein Eng. (1996), which is incorporated herein by reference. In another example, the multimerization domain is a cystine knot. For example, the cystine knot comprises up to 60 amino acids in length including a core domain of three or more interwoven disulfide bonds. In a further example, the multimerization domain is an antibody Fc region (e.g., as described herein).

In one example, the multimerized sCR1 variant is linear. In one example, the multimerized sCR1 variant is circular. For example, the multimerized sCR1 variant can comprise a sortase enzyme cleavage site, as described in Popp, M. W. et al. PNAS (2011), incorporated herein by reference.

In one example, the sCR1 variant for use in the present disclosure comprises at least two sialylated glycans (e.g., di-, tri- or tetra-sialylated glycans). For example, a composition for use in any method described herein comprises a sialylated sCR1 variant glycoform. In one example, a sialylated sCR1 variant glycoform for use in any method described herein comprises di-, tri- or tetra-sialylated glycoforms. Methods for producing variant sCR1 glycoforms comprising at least two sialylated glycans (e.g., di-, tri- or tetra-sialylated glycans), will be apparent to the skilled person and/or described herein.

Exemplary methods for determining the biological activity of the sCR1 variant of the disclosure will be apparent to the skilled person and/or described herein. For example, methods for determining inhibitory activity of the classical, lectin and/or alternative pathway are described herein.

### Proteins Comprising Antigen Binding Domains

The present disclosure provides a sCR1 variant conjugated to a protein comprising an antigen binding domain that binds to a target and inhibits signaling by or via the target. For example, the protein comprises at least a V_{H} and a V_{L}, wherein the V_{H} and V_{L} bind to form a Fv comprising an antigen binding domain.

In one example, the antigen binding domain binds to or specifically binds to the target and neutralises the signalling.

In one example, the protein binds to or specifically binds to G-CSF or G-CSFR and neutralizes G-CSF signalling. For example, the protein binds to or specifically binds to G-CSF and neutralizes G-CSF signalling. In another example, the protein binds to or specifically binds to G-CSFR and neutralizes G-CSF signalling.

The present disclosure also provides a sCR1 variant conjugated to a protein comprising an antigen binding domain that binds to a blood coagulation factor. For example, the protein comprises at least a V_{H} and a V_{L}, wherein the V_{H} and V_{L} bind to form a Fv comprising an antigen binding domain.

In one example, the antigen binding domain binds to or specifically binds to the blood coagulation factor and neutralises the activity. For example, the blood coagulation factor is Factor XII and/or activated Factor XII (FXIIa). In another example, the blood coagulation factor is Factor XI and/or activated Factor XI (FXIa).

In one example, the protein binds to or specifically binds to Factor XII and/or activated Factor XII (FXIIa) and antagonises activation of Factor XII and/or Factor XIIa and/or antagonises activity of Factor XII and/or Factor XIIa. For example, the protein binds to or specifically binds to Factor XII and antagonises activity of Factor XII and/or Factor XIIa. For example, the protein binds to or specifically binds to Factor XII and inhibits the activation of Factor XII to Factor XIIa (i.e., antagonises activation).

In another example, the protein binds to or specifically binds to Factor XI and/or activated Factor XI (FXIa) and antagonises activation of Factor XI and/or Factor XIa and/or antagonises activity of Factor XI and/or Factor XIa. For example, the protein binds to or specifically binds to Factor XI and antagonises activity of Factor XI and/or Factor XIa. For example, the protein binds to or specifically binds to Factor XI and inhibits the activation of Factor XI to Factor XIa (i.e., antagonises activation).

### Antibodies and Antibody Fragments

In one example, the protein comprising an antigen binding domain that binds to a target and inhibits signaling by or via the target is an antibody or antigen binding fragment. For example, the protein is an antibody or antigen binding fragment that binds to G-CSF or G-CSFR. For example, the protein is an antibody or antigen binding fragment that binds to G-CSFR. In another example, the protein is an antibody or antigen binding fragment that binds to G-CSF.

In one example, the protein comprising an antigen binding domain that binds to a blood coagulation factor is an antibody or antigen binding fragment. For example, the protein is an antibody or antigen binding fragment that binds to Factor XII or activated Factor XII (FXIIa). For example, the protein is an antibody or antigen binding fragment that binds to Factor XII. In another example, the protein is an antibody or antigen binding fragment that binds to activated Factor XII (FXIIa).

Methods for generating antibodies are known in the art and/or described in Harlow and Lane (editors) Antibodies: A Laboratory Manual, Cold Spring Harbor Laboratory, (1988). Generally, in such methods G-CSFR or G-CSF (e.g., hG-CSFR or hG-CSF) or Factor XII (e.g., hFXII) or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present disclosure may be a synthetic antibody. For example, the antibody is a chimeric antibody, a humanized antibody, a human antibody or a de-immunized antibody.

In one example, an antibody described herein is a chimeric antibody. The term “chimeric antibody” refers to antibodies in which a portion of the heavy and/or light chain is identical with or homologous to corresponding sequences in antibodies derived from a particular species (e.g., murine, such as mouse) or belonging to a particular antibody class or subclass, while the remainder of the chain(s) is identical with or homologous to corresponding sequences in antibodies derived from another species (e.g., primate, such as human) or belonging to another antibody class or subclass. Methods for producing chimeric antibodies are described in, e.g., U.S. Pat. No. 4,816,567; and U.S. Pat. No. 5, 807,715.

The antibodies of the present disclosure may be humanized or human.

The term “humanized antibody” shall be understood to refer to a subclass of chimeric antibodies having an antigen binding site or variable region derived from an antibody from a non-human species and the remaining antibody structure based upon the structure and/or sequence of a human antibody. In a humanized antibody, the antigen-binding site generally comprises the complementarity determining regions (CDRs) from the non-human antibody grafted onto appropriate FRs in the variable regions of a human antibody and the remaining regions from a human antibody. Antigen binding sites may be wild-type (i.e., identical to those of the non-human antibody) or modified by one or more amino acid substitutions. In some instances, FR residues of the human antibody are replaced by corresponding non-human residues.

Methods for humanizing non-human antibodies or parts thereof (e.g., variable regions) are known in the art. Humanization can be performed following the method of U.S. Pat. No. 5,225,539, or U.S. Pat. No. 5,585,089. Other methods for humanizing an antibody are not excluded.

The term “human antibody” as used herein refers to antibodies having variable regions (e.g. V_{H}, V_{L}) and, optionally constant regions derived from or corresponding to sequences found in humans, e.g. in the human germline or somatic cells.

Additional exemplary antibodies or antigen binding fragments thereof for use in the present disclosure are described herein or known in the art and include:
a synhumanized antibody or fragment thereof, e.g., an antibody that includes a variable region comprising FRs from a New World primate antibody variable region and CDRs from a non-New World primate antibody variable region (e.g., produced by methods described in WO2007019620).
a primatized antibody or fragment thereof, e.g., an antibody comprising variable region(s) from an antibody generated following immunization of a non-human primate (e.g., a cynomolgus macaque) (e.g., produced by methods described in U.S. Pat. No. 6,113,898).
a deimmunized antibody or antigen binding fragment thereof, e.g., antibodies and fragments that have one or more epitopes, e.g., B cell epitopes or T cell epitopes removed (i.e., mutated) to thereby reduce the likelihood that a subject will raise an immune response against the antibody or protein (e.g., as described in WO2000034317 and WO2004108158).
a bispecific antibody or fragment thereof, e.g., an antibody comprising two types of antibodies or antibody fragments (e.g., two half antibodies) having specificities for different antigens or epitopes (e.g., as described in U.S. Pat. No. 5,731,168).

Exemplary human antibodies that bind to G-CSF or G-CSFR are described herein and include C1.2 and C1.2G and/or variable regions thereof. These human antibodies provide an advantage of reduced immunogenicity in a human compared to non-human antibodies. Exemplary antibodies are described in WO2012171057, which is incorporated herein by reference. Other antibodies suitable for use in accordance with the methods of the disclosure include those described in WO2018/145206.

Exemplary human Factor XII antibodies are described herein and include 3F7, 3F7G and affinity matured 3F7 and/or variable regions thereof. A further exemplary antibody is the anti-FXII antibody garadacimab. These human antibodies provide an advantage of reduced immunogenicity in a human compared to non-human antibodies. Exemplary antibodies are described in WO2013/014092, WO2009/067660, WO2009/154461, WO2010/080623, WO2013/167669, WO2016/207858, WO2017/015619, WO2017/162791, WO2017/127468 and WO2017/218371, which are incorporated herein by reference. Additional antibodies and proteins comprising variable regions are described in WO2006/066878, and in Rayon et al., Blood 86: 4134-43 (1995).

### Antigen-Binding Domain Containing Proteins

In some examples, the protein of the disclosure is a protein that is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, Mass.; see, e.g., U.S. Pat. No. 6,248,516).

In some examples, a protein of the disclosure is or comprises a diabody, triabody, tetrabody or higher order protein complex such as those described in WO1998/044001 and/or WO1994/007921.

In some examples, the protein of the disclosure is or comprises an scFv. The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

In some examples, the protein of the disclosure is or comprises a heavy chain antibody. Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR). A general description of heavy chain antibodies from camelids and the variable regions thereof and methods for their production and/or isolation and/or use is found inter alia in the following references WO1994/04678, WO1997/49805 and WO 1997/49805. A general description of heavy chain antibodies from cartilaginous fish and the variable regions thereof and methods for their production and/or isolation and/or use is found inter alia in WO2005/118629.

### Other Antibodies and Antibody Fragments

The present disclosure also contemplates other antibodies and antibody fragments, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab3 (e.g., as described in EP19930302894).

### Stabilized Proteins

Proteins of the present disclosure can comprise an IgG4 constant region or a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

### Conjugation

Methods for conjugation of the sCR1 variant and protein comprising an antigen binding domain will be apparent to the skilled person and/or described herein. All forms and methods of conjugation (i.e., binding) are contemplated by the present disclosure, including, for example, direct conjugation between the sCR1 variant and protein comprising an antigen binding domain as described herein or indirect binding (e.g., by virtue of a linker between the sCR1 variant and the protein comprising an antigen binding domain) In one example, the conjugate is formed by a chemical conjugation (e.g., by an amine bond or disulphide bond) or by genetic fusion.

In one example, the sCR1 variant of the present disclosure is conjugated to a protein comprising an antigen binding domain that binds to a target and inhibits signaling by or via the target. In another example, the sCR1 variant of the present disclosure is conjugated to a protein comprising an antigen binding domain that binds to a blood coagulation factor. For example, the protein can be directly or indirectly bound to the sCR1 variant (e.g., can comprise a linker in the case of indirect binding).

In one example, the sCR1 variant is conjugated to the protein comprising an antigen binding domain by an amine bond.

In one example, the present disclosure provides a fusion protein comprising the sCR1 variant and the protein comprising an antigen binding domain. For example, the protein comprising an antigen binding domain is positioned at N-terminus of the sCR1 variant, C-terminus of the sCR1 variant or any combination thereof.

In one example, the sCR1 variant is conjugated to the protein comprising an 20 antigen binding domain via a linker. For example, the linker is a peptide linker.

In one example, the linker is a flexible linker. A “flexible” linker is an amino acid sequence which does not have a fixed structure (secondary or tertiary structure) in solution. Such a flexible linker is therefore free to adopt a variety of conformations. Flexible linkers suitable for use in the present disclosure are known in the art. An example of a flexible linker for use in the present invention is the linker sequence SGGGGS/GGGGS/GGGGS or (Gly4Ser)3. Flexible linkers are also disclosed in WO1999/045132.

The linker may comprise any amino acid sequence that does not substantially hinder interaction of the binding region with its target. Preferred amino acid residues for 30 flexible linker sequences include, but are not limited to, glycine, alanine, serine, threonine proline, lysine, arginine, glutamine and glutamic acid.

The linker sequences between the binding regions preferably comprise five or more amino acid residues. The flexible linker sequences according to the present disclosure consist of 5 or more residues, preferably, 5, 6, 7, 8, 9, 10, 11, 12, 13, 14, 15, 16, 17, 18, 19 or 20 or 25 or 30 or more residues. In a highly preferred embodiment of the invention, the flexible linker sequences consist of 5, 7, 10, 13, 15, 16, 20 or 30 residues.

In one example, the flexible linker has an amino acid sequence according to SEQ ID NO: 31, i.e., GSGGSGGSGGSGS (GS13).

In one example, the flexible linker has an amino acid sequence according to SEQ ID NO: 32, i.e., GGGGSGGGGSGGGGS (GS15).

In one example, the flexible linker has an amino acid sequence according to SEQ ID NO: 33, i.e., SGGGGSGGGGSGGGGS (GS16).

In one example, the flexible linker has an amino acid sequence according to SEQ ID NO: 55, i.e., GGGGSGGGGSGGGGGS (GS16).

In one example, the flexible linker has an amino acid sequence according to SEQ ID NO: 34, i.e., GGGGSGGGGSGGGGSGGGGS (GS20).

In one example, the flexible linker has an amino acid sequence according to SEQ ID NO: 35, i.e., SGGSGGSGGSGGSGGSGGSGGSGGSGGSGS (GS30).

Exemplary proteins comprising an antigen binding domain that can be conjugated to a sCR1 variant of the disclosure and methods for such conjugation are known in the art and described herein.

Inhibiting Complement Activity and/or G-CSF Activity and/or Factor XII/XIIa Activity and/or Factor XI/XIa Activity

The present disclosure provides, for example, a method of inhibiting complement activity in a subject comprising administering to the subject a soluble complement receptor type 1 (sCR1) conjugate of the present disclosure.

The present disclosure also provides, for example, a method of inhibiting G-CSF activity in a subject comprising administering to the subject a soluble complement receptor type 1 (sCR1) conjugate of the present disclosure.

The present disclosure also provides, for example, a method of inhibiting complement activity and G-CSF activity in a subject comprising administering to the subject a soluble complement receptor type 1 (sCR1) conjugate of the present disclosure.

The present disclosure also provides, for example, a method of antagonising activity of Factor XII and/or Factor XIIa and/or antagonising activation of Factor XII and/or Factor XIIa in a subject comprising administering to the subject a soluble complement receptor type 1 (sCR1) conjugate of the present disclosure.

The present disclosure also provides, for example, a method of inhibiting complement activity and/antagonising activity of Factor XII and/or Factor XIIa and/or antagonising activation of Factor XII and/or Factor XIIa in a subject comprising administering to the subject a soluble complement receptor type 1 (sCR1) conjugate of the present disclosure.

The present disclosure also provides, for example, a method of antagonising activity of Factor XI and/or Factor XIa and/or antagonising activation of Factor XI and/or Factor XIa in a subject comprising administering to the subject a soluble complement receptor type 1 (sCR1) conjugate of the present disclosure.

The present disclosure also provides, for example, a method of inhibiting complement activity and/antagonising activity of Factor XI and/or Factor XIa and/or antagonising activation of Factor XI and/or Factor XIa in a subject comprising administering to the subject a soluble complement receptor type 1 (sCR1) conjugate of the present disclosure.

The present disclosure also provides a method of treating or preventing a disease or condition in a subject, the method comprising administering the sCR1 conjugate or composition comprising the sCR1 conjugate of the present disclosure to a subject. In one example, the present disclosure provides a method of treating a disease or condition in a subject in need thereof.

The present disclosure also provides for use of a sCR1 conjugate or composition comprising the sCR1 conjugate of the present disclosure for treating or preventing a disease or condition in a subject.

In one example, the present disclosure provides for use of a sCR1-anti-G-CSFR conjugate of the present disclosure for treating a disease or condition in a subject in need thereof.

In one example, the present disclosure also provides for use of a sCR1-anti-Factor XII conjugate of the present disclosure for treating a disease or condition in a subject in need thereof. In another example, the present disclosure provides for use of a sCR1-anti-Factor XIIa conjugate of the present disclosure for treating a disease or condition in a subject in need thereof. In a further example, the present disclosure provides for use of a sCR1-anti-Factor XII/XIIa conjugate of the present disclosure for treating a disease or condition in a subject in need thereof.

In one example, the present disclosure provides for use of a sCR1-anti-Factor XI conjugate of the present disclosure for treating a disease or condition in a subject in need thereof. In another example, the present disclosure provides for use of a sCR1-anti-Factor XIa conjugate of the present disclosure for treating a disease or condition in a subject in need thereof. In a further example, the present disclosure provides for use of a sCR1-anti-Factor XI/XIa conjugate of the present disclosure for treating a disease or condition in a subject in need thereof.

In one example, the method comprises inhibiting activity in the classical pathway, the lectin pathway and/or the alternative complement pathway. For example, the method comprises administering a sCR1 conjugate of the present disclosure to inhibit activation of the classical complement pathway. In another example, the method comprises administering a sCR1 conjugate of the present disclosure to inhibit activation of the lectin pathway. In a further example, the method comprises administering a sCR1 conjugate of the present disclosure to inhibit activation of the alternative complement pathway.

In one example, the method comprises inhibiting activity in the extrinsic complement pathway. For example, the method comprises administering a sCR1 conjugate of the present disclosure to inhibit activation of the extrinsic complement pathway.

In one example, the disease or condition is a complement-mediated disorder, a neutrophil-mediated disorder and/or a blood coagulation disorder.

In one example, the subject suffers from a complement-mediated disorder, a neutrophil-mediated disorder and/or a blood coagulation disorder. The complement-mediated disorder, neutrophil-mediated disorder and/or blood coagulation disorder can be inherited or acquired.

In one example, the complement-mediated disorder, the neutrophil-mediated disorder and/or the blood coagulation disorder is selected from the group consisting of an inflammatory joint condition, inflammatory arthritis, inflammatory eye condition, inflammatory lung condition, inflammatory neurological condition, autoimmune intestinal disorders, psoriasis, cancer (including angiogenesis thereof) or metastasis thereof, solid organ transplantation (e.g., lung and/or renal transplantation (including antibody mediated rejection)), ischemia reperfusion injury before, during or after 25 transplantation, delayed graft function, asthma and exacerbated forms thereof, neutrophilic dermatosis and a neutrophilic skin lesion, ischemic stroke with reperfusion, neurotraumatic disorder, somatic trauma, ischemia-reperfusion injury (IRI, including myocardial IRI, intestinal IRI, liver IRI and/or pancreatic IRI), venous, arterial or capillary thrombus formation, thrombus formation in the heart, contact-mediated thrombo-inflammation, thrombus formation during and/or after contacting blood of a human or animal subject with artificial surfaces, interstitial lung disease, inflammation, a neurological inflammatory disease, fibrinolysis, angiogenesis, a thrombo-inflammatory disease, a disease related to FXII/FXII-induced kinin formation, atrial fibrillation, acute coronary syndromes (ACS), acute limb ischemia, acute respiratory distress syndrome (ARDS; or acute lung injury) and lupus nephritis (including acute lupus nephritis or chronic lupus nephritis).

In one example, the complement-mediated disorder, the neutrophil-mediated disorder and/or the blood coagulation disorder is selected from the group consisting of paroxysmal nocturnal haemoglobinuria (PNH), atypical haemolytic uraemic syndrome (aHUS), thrombocytopenic purpura (TTP), thrombotic microangiopathy, C3 glomerulopathy, membranoproliferative glomerulonephritis (including anti-Thy 1 glomerulonephritis, anti-conA diffuse proliferative glomerulonephritis and/or passive heymann nephritis), and/or, Guillain-Barré syndrome, myasthenia gravis (including autoimmune gyasthenia gravis, demyelinating allergic encephalomyelitis, IgG immune complex alveolitis, reverse passive arthus reaction), systemic lupus erythematosus (SLE), IgA nephropathy, autoimmune haemolytic anemia, pemphigus (including pemphigus vulgaris), pemphigoid (including bullous pemphigoid), anti-phospholipid syndrome, polytrauma, haemodialysis, post-infection HUS, macular degeneration, ANCA-associated vasculitis, atherosclerosis, mood disorders, chronic inflammatory demyelinating polyneuropathy (CIDP), anaphylaxis, cerebral malaria, dermatomyositis, osteoarthritis, dementia, glaucoma, diabetic angiopathy, myocardial infarction, anti-glomerular basement membrane (GBM) nephritis (or Goodpasture's syndrome), autoimmune epilepsy, dermatitis herpetiformis, eosinophilic granulomatosis with polyangiitis (EGPA; or Churg-Strauss syndrome), Sjögren's syndrome and Sjögren's syndrome vasculitis.

Methods for diagnosis of a complement-mediated disorder, a neutrophil mediated disorder and/or a blood coagulation disorder will be readily apparent to the skilled person and include, for example, haemolytic classical complement pathway (CH-50) test, haemolytic alternative complement pathway (AP-50) test, screening for immune complex diseases, antinuclear serology to test for lupus, urinalysis and complete blood count (CBC).

In one example, the method comprising inhibiting G-CSF activity in the subject. For example, the method comprises administering a sCR1 conjugate of the present disclosure to inhibit activation of G-CSF signalling pathway.

In one example, the sCR1 conjugate or composition comprising the sCR1 conjugate of the present disclosure is administered to the subject in an amount sufficient to reduce the number of neutrophils in a subject without inducing neutropenia.

In one example, the method comprises inhibiting Factor XII and/or Factor XIIa activity in the subject. In one example, the method comprises administering a sCR1 conjugate of the present disclosure to inhibit activation of Factor XII and/or Factor XIIa activity. For example, the sCR1 conjugate of the present disclosure inhibits the activation of Factor XII to Factor XIIa.

In one example, the method comprises antagonising activity of Factor XII and/or Factor XIIa and/or antagonising activation of Factor XII and/or Factor XIIa in the subject.

In one example, the method comprises inhibiting Factor XI and/or Factor XIa activity in the subject. In one example, the method comprises administering a sCR1 conjugate of the present disclosure to inhibit activation of Factor XI and/or Factor XIa. For example, the sCR1 conjugate of the present disclosure inhibits the activation of Factor XI to Factor XIa.

In one example, the sCR1 conjugate or composition comprising the sCR1 conjugate of the present disclosure is administered to the subject in amount sufficient to inhibit the amidolytic activity of human Factor XIIa.

In one example, the subject is at risk of developing a complement-mediated disorder, a neutrophil-mediated disorder and/or a blood coagulation disorder. A subject is at risk if he or she has a higher risk of developing a complement-mediated disorder, a neutrophil-mediated disorder and/or a blood coagulation disorder than a control population. The control population may include one or more subjects selected at random from the general population (e.g., matched by age, gender, race and/or ethnicity) who have not suffered from or have a family history of a complement-mediated disorder, a neutrophil-mediated disorder and/or a blood coagulation disorder. A subject can be considered at risk for a complement-mediated disorder, a neutrophil-mediated disorder and/or a blood coagulation disorder if a “risk factor” associated with a complement-mediated disorder, a neutrophil-mediated disorder and/or a blood coagulation disorder is found to be associated with that subject. A risk factor can include any activity, trait, event or property associated with a given disorder, for example, through statistical or epidemiological studies on a population of subjects. A subject can thus be classified as being at risk for a complement-mediated disorder, a neutrophil-mediated disorder and/or a blood coagulation disorder even if studies identifying the underlying risk factors did not include the subject specifically.

In one example, the subject is at risk of developing a complement-mediated disorder, a neutrophil-mediated disorder and/or a blood coagulation disorder and the sCR1 conjugate is administered before or after the onset of symptoms of a complement-mediated disorder, a neutrophil-mediated disorder and/or a blood coagulation disorder. In one example, the sCR1 conjugate is administered before the onset of symptoms of a complement-mediated disorder, a neutrophil-mediated disorder and/or a blood coagulation disorder. In one example, the sCR1 conjugate is administered after the onset of symptoms of a complement-mediated disorder, a neutrophil-mediated disorder and/or a blood coagulation disorder. In one example, the sCR1 conjugate of the present disclosure is administered at a dose that alleviates or reduces one or more of the symptoms of a complement-mediated disorder, a neutrophil-mediated disorder and/or a blood coagulation disorder in a subject at risk.

The methods of the present disclosure can be readily applied to any form of complement-mediated disorder, neutrophil-mediated disorder and/or blood coagulation disorder in a subject.

In one example, a method of the disclosure reduces any symptom of a complement-mediated disorder, a neutrophil-mediated disorder and/or a blood coagulation disorder known in the art and/or described herein.

As will be apparent to the skilled person a “reduction” in a symptom of a complement-mediated disorder, a neutrophil-mediated disorder and/or a blood coagulation disorder in a subject will be comparative to another subject who also suffers from a complement-mediated disorder, a neutrophil-mediated disorder and/or a blood coagulation disorder but who has not received treatment with a method described herein. This does not necessarily require a side-by-side comparison of two subjects. Rather population data can be relied upon. For example, a population of subjects suffering from a complement-mediated disorder, a neutrophil-mediated disorder and/or a blood coagulation disorder who have not received treatment with a method described herein (optionally, a population of similar subjects to the treated subject, e.g., age, weight, race) are assessed and the mean values are compared to results of a subject or population of subjects treated with a method described herein.

In the case of a complement-mediated disorder, a neutrophil-mediated disorder and/or a blood coagulation disorder that is an ischemia-reperfusion injury due to or associated with organ transplantation, the sCR1 conjugate of the disclosure or composition comprising the sCR1 conjugate can be administered before, during or after transplantation. In some examples, the sCR1 conjugate or composition is administered to an organ transplantation donor. In other examples, the sCR1 conjugate or composition is administered to the subject, wherein the subject is an organ transplantation recipient. In one example, the sCR1 conjugate or composition is administered to a harvested organ ex vivo, prior to organ transplantation. For example, the harvested organ can be perfused or infused with a solution comprising the sCR1 conjugate or composition prior to transplantation.

In one example, the organ transplantation is solid organ transplantation. For example, the solid organ transplantation is lung transplantation. In another example, the solid organ transplantation is renal transplantation.

It will be apparent to the skilled person from the foregoing, that the present disclosure provides a method of organ transplantation or for improving outcome of an organ transplantation or improving function of a transplanted organ or for preventing delayed graft function, the method comprising administering a sCR1 conjugate or composition to an organ transplant donor prior to collection of the organ; collecting the organ and transplanting the organ into an organ transplant recipient.

The present disclosure also provides a method for preparing a transplant organ from an organ donor to improve organ function in an organ transplant recipient, the method comprising administering to the organ donor a sCR1 conjugate or composition prior to collection of the organ.

The present disclosure additionally provides a method for preventing organ transplant rejection, the method comprising administering to an organ donor a sCR1 conjugate or composition prior to collection of the organ, collecting the organ and transplanting the organ into an organ transplant recipient.

In some examples, the method additionally comprises administering the sCR1 conjugate or composition to the organ transplant recipient. For example, the sCR1 conjugate or composition is administered to the organ transplant recipient before the transplant or at the time of transplanting the organ (i.e., during transplantation).

The present disclosure also provides a method of organ transplantation or for improving outcome of an organ transplantation or improving function of a transplanted organ or for preventing delayed graft function, the method comprising administering a sCR1 conjugate or composition to an organ transplant recipient prior to transplanting the organ and then transplanting the organ into the organ transplant recipient.

In one example, the organ transplant donor is brain dead. For example, the organ donor is alive by virtue of life support but is brain dead.

In one example of the disclosure, the sCR1 conjugate or composition is administered before reperfusion, for example, in the case of an organ transplant, the sCR1 conjugate or composition is administered to an organ transplant recipient prior to reperfusion of the transplanted organ (e.g., the sCR1 conjugate or composition is administered prior to the transplantation or during the transplantation but before reperfusion).

In the case of administration to a brain dead donor, the sCR1 conjugate or composition can be administered at any time between brain death and organ collection. In some examples, the sCR1 conjugate or composition is administered to a harvested organ ex vivo, prior to organ transplantation. For example, the harvested organ can be perfused or infused with a solution comprising the sCR1 conjugate or composition prior to transplantation.

Assaying Activity of a sCR1 Conjugate

sCR1 variants and/or conjugates of the present disclosure are readily screened for biological activity, e.g., as described below.

### Measuring Complement Activity

In one example, complement activity is measured using an enzyme immunoassay (e.g., a Wieslab® complement assay kit). For example, complement inhibitory activity is determined using labelled antibodies specific for an antigen or an epitope produced during complement activation (e.g., C5b-9 or an epitope present in C5b-9). In one example, the wells of a microtitre plate are coated with specific activators of the classical, lectin or alternative pathway. In another example, the sCR1 variant and/or conjugate is incubated with normal human serum and appropriate assay diluent (i.e., a diluent comprising appropriate components to ensure specific activation of the classical, lectin or alternative pathway) and added to microtitre plate wells coated with specific activators of the classical, lectin or alternative pathway and the amount of C5b-9 complex formed is detected using a specific alkaline phosphatase labelled antibody to the C5b-9. In one example, the amount of complement activation product (i.e., C5b-9) produced is proportional to the functional activity of the complement pathway. In one example, the half maximal inhibitor concentration (i.e., IC₅₀) is determined. For example, the IC₅₀ of the sCR1 variant is determined and compared to the IC₅₀ of a sCR1 comprising a sequence set forth in SEQ ID NO: 2. In another example, the IC₅₀ of the sCR1 conjugate is determined and compared to the IC₅₀ of a sCR1 conjugate comprising a sCR1 comprising a sequence set forth in SEQ ID NO: 2.

In another example, complement inhibitory activity is determined using a hemolysis assay (e.g., classical pathway (i.e., CH50) and alternative pathway (ApH50) inhibition assays). The CH50 assay is a method for measuring the total classical complement activity in serum. This test is a lytic assay, which uses antibody-sensitized erythrocytes as the activator of the classical complement pathway and human serum as complement source. The percent hemolysis can be determined, for example, using a spectrophotometer. The CH50 assay provides an indirect measure of terminal complement complex (TCC) formation, since the TCC themselves are directly responsible for the hemolysis that is measured. The assay is well known. Briefly, to assess the inhibition of the classical complement pathway, pre-diluted human serum is pre-incubated in microassay wells, together with serially diluted sCR1 variants and/or conjugates. Next, antibody-sensitized erythrocytes (e.g., sheep erythrocytes sensitized with rabbit anti-sheep antibodies) are added. After centrifugation, free haemoglobin is measured in the supernatants, using a spectrophotometer. The decrease in free haemoglobin reflects the inhibition of TCC-mediated erythrocyte lysis. sCR1-mediated inhibition is then calculated relative to erythrocytes which were incubated with human serum only (100% lysis sample).

Complement inhibition can also be evaluated based on any methods known in the art, including for example, in vitro zymosan assays, assays for lysis of erythrocytes, antibody or immune complex activation assays, alternative pathway activation assays, and lectin pathway activation assays.

Binding to G-CSFR and Mutants Thereof Factor XII and/or Factor XIIa and/or Factor XI and/or Factor XIa

It will be apparent to the skilled artisan from the disclosure herein that some proteins of the present disclosure bind to the ligand binding domain of hG-CSFR and to specific mutant forms of the ligand binding domain of hG-CSFR (e.g., SEQ ID NO: 48 without or with certain point mutations) and/or bind to both human and cynomolgus monkey G-CSFR.

It will also be apparent to the skilled artisan from the disclosure herein that some proteins of the present disclosure bind to the ligand binding domain of Factor XII and/or activated Factor XII (i.e., Factor XIIa) and/or Factor XI and/or activated Factor XI (i.e., Factor XIa).

Methods for assessing binding to a protein are known in the art, e.g., as described in Scopes (In: Protein purification: principles and practice, Third Edition, Springer Verlag, 1994). Such a method generally involves labeling the protein and contacting it with immobilized compound. Following washing to remove non-specific bound protein, the amount of label and, as a consequence, bound protein is detected. Of course, the protein can be immobilized and the compound that inhibits G-CSF signaling or binds to Factor XII and/or Factor XIIa and/or Factor XI and/or Factor XIa labeled. Panning-type assays can also be used. Alternatively, or additionally, surface plasmon resonance assays can be used.

The assays described above can also be used to detect the level of binding of a protein of the present disclosure to hG-CSFR or a ligand binding domain thereof (e.g., SEQ ID NO: 48) or mutant form thereof or to Factor XII and/or Factor XIIa and/or Factor XI and/or Factor Xia or a ligand binding domain thereof. Methods of detecting the level of binding will be apparent to the skilled person and/or described herein. For example, the level of binding is determined using a biosensor.

### Epitope Mapping

In another example, the epitope bound by a protein described herein is mapped. Epitope mapping methods will be apparent to the skilled artisan. For example, a series of overlapping peptides spanning the hG-CSFR sequence or a region thereof comprising an epitope of interest, e.g., peptides comprising 10-15 amino acids are produced. The protein is then contacted to each peptide and the peptide(s) to which it binds determined. This permits determination of peptide(s) comprising the epitope to which the protein binds. If multiple non-contiguous peptides are bound by the protein, the protein may bind a conformational epitope.

Alternatively, or in addition, amino acid residues within hG-CSFR are mutated, e.g., by alanine scanning mutagenesis, and mutations that reduce or prevent protein binding are determined. Any mutation that reduces or prevents binding of the protein is likely to be within the epitope bound by the protein.

A further method is exemplified herein, and involves binding hG-CSFR or a region thereof to an immobilized protein of the present disclosure and digesting the resulting complex with proteases. Peptide that remains bound to the immobilized protein are then isolated and analyzed, e.g., using mass spectrometry, to determine their sequence.

A further method involves converting hydrogens in hG-CSFR or a region thereof to deutrons and binding the resulting protein to an immobilized protein of the present disclosure. The deutrons are then converted back to hydrogen, the hG-CSFR or region thereof isolated, digested with enzymes and analyzed, e.g., using mass spectrometry to identify those regions comprising deutrons, which would have been protected from conversion to hydrogen by the binding of a protein described herein.

Optionally, the dissociation constant (Kd) of a protein for hG-CSFR or an epitope thereof is determined. The “Kd” or “Kd value” for a hG-CSFR binding protein is in one example measured by a radiolabeled or fluorescently-labeled hG-CSFR binding assay. This assay equilibrates the protein with a minimal concentration of labeled G-CSFR in the presence of a titration series of unlabeled hG-CSFR. Following washing to remove unbound hG-CSFR, the amount of label is determined, which is indicative of the Kd of the protein.

According to another example the Kd or Kd value is measured by using surface plasmon resonance assays, e.g., using BIAcore surface plasmon resonance (BIAcore, Inc., Piscataway, N.J.) with immobilized hG-CSFR or a region thereof.

In some examples, proteins having a similar Kd or a higher Kd than C1.2 or C1.2G are selected, because they are likely to compete for binding to hG-CSFR.

Measuring Factor XII/XIIa and/or Factor XI/XIa Activity

Methods for assessing the inhibitory activity of a protein are known in the art, and include for example a chromogenic assay. Chromogenic assays for measuring inhibitory activity are known in the art.

In one example, assay buffer is pre-mixed with Factor XIIa and/or Factor XIa. The conjugate of the present disclosure is added followed by chromogenic substrate. Following cessation of the chromogenic reaction, the inhibitory activity of the conjugate is assessed.

### Determining Competitive Binding

Assays for determining a protein that competitively inhibits binding of antibodies C1.2, C1.2G, 3F7 and/or 3F7G (or any other antibody described herein) will be apparent to the skilled artisan. For example, C1.2, C1.2G, 3F7 or 3F7G is conjugated to a detectable label, e.g., a fluorescent label or a radioactive label. The labeled antibody and the test protein are then mixed and contacted with hG-CSFR or a region thereof (e.g., a polypeptide comprising SEQ ID NO: 48) or Factor XII or a region thereof or a cell expressing same. The level of labeled C1.2 or C1.2G (or 3F7 or 3F7G) is then determined and compared to the level determined when the labeled antibody is contacted with the hG-CSFR (or with Factor XII), region or cells in the absence of the protein. If the level of labeled C1.2 or C1.2G (or 3F7 or 3F7G) is reduced in the presence of the test protein compared to the absence of the protein, the protein is considered to competitively inhibit binding of C1.2 or C1.2G to hG-CSFR (or 3F7 or 3F7G to Factor XII).

Optionally, the test protein is conjugated to a different label to C1.2 or C1.2G. This alternate labeling permits detection of the level of binding of the test protein to hG-CSFR or the region thereof or the cell.

Optionally, the test protein is conjugated to different label to 3F7 or 3F7G. This alternate labeling permits detection of the level of binding of the test protein to Factor XII or the region thereof or the cell.

In another example, the protein is permitted to bind to hG-CSFR or a region thereof (e.g., a polypeptide comprising SEQ ID NO: 48) or a cell expressing same prior to contacting the hG-CSFR, region or cell with C1.2 or C1.2G. A reduction in the amount of bound C1.2 or C1.2G in the presence of the protein compared to in the absence of the protein indicates that the protein competitively inhibits C1.2 or C1.2G binding to hG-CSFR. A reciprocal assay can also be performed using labeled protein and first allowing C1.2 or C1.2G to bind to G-CSFR. In this case, a reduced amount of labeled protein bound to hG-CSFR in the presence of C1.2 or C1.2G compared to in the absence of C1.2 or C1.2G indicates that the protein competitively inhibits binding of C1.2 or C1.2G to hG-CSFR.

Any of the foregoing assays can be performed with a mutant form of hG-CSFR and/or SEQ ID NO: 48 and/or a ligand binding region of hG-CSFR to which C1.2 or C1.2G binds, e.g., as described herein.

In another example, the protein is permitted to bind to Factor XII or a region thereof or a cell expressing same prior to contacting the Factor XII, region or cell with 3F7 or 3F7G. A reduction in the amount of bound 3F7 or 3F7G in the presence of the protein compared to in the absence of the protein indicates that the protein competitively inhibits 3F7 or 3F7G binding to Factor XII. A reciprocal assay can also be performed using labeled protein and first allowing 3F7 or 3F7G to bind to Factor XII. In this case, a reduced amount of labeled protein bound to Factor XII in the presence of 3F7 or 3F7G compared to in the absence of 3F7 or 3F7G indicates that the protein competitively inhibits binding of 3F7 or 3F7G to Factor XII.

### Determining Inhibition of G-CSF Signaling

In some examples of the present disclosure, a sCR1 conjugate (e.g., a sCR1 variant conjugated to a protein comprising an antigen binding domain) is capable of neutralizing hG-CSFR signaling.

Various assays are known in the art for assessing the ability to neutralize signaling of a ligand through a receptor.

In one example, the protein that inhibits G-CSF signaling reduces or prevents G-CSF binding to the hG-CSFR. These assays can be performed as a competitive binding assay as described herein using labeled G-CSF and/or labeled protein.

In one example, the sCR1 conjugate comprising a protein that inhibits G-CSF signaling reduces formation of CFU-G when CD34⁺ bone marrow cells are cultured in the presence of G-CSF. In such assays, CD34⁺ bone marrow cells are cultured in a semi-solid cell culture medium in the presence of G-CSF (e.g., about 10 ng/ml cell culture medium) and, optionally stem cell factor (e.g., about 10 ng/ml cell culture medium) in the presence or absence of a test compound (e.g., a sCR1 conjugated to a protein comprising an antigen binding domain that binds G-CSFR). After a sufficient time for granulocyte clones (CFU-G) to form, the number of clones or colonies is determined. A reduction in the number of colonies in the presence of the compound compared to in the absence of the compound indicates that the compound neutralizes G-CSF signaling.

In a further example, the sCR1 conjugate comprising a protein that inhibits G-CSF signaling reduces proliferation of cells (e.g., BaF3 cells) expressing hG-CSFR which are cultured in the presence of G-CSF. Cells are cultured in the presence of G-CSF (e.g., 0.5 ng/ml) and the presence or absence of a test compound (e.g., a sCR1 variant conjugated to a protein that inhibits G-CSF signaling). Methods for assessing cell proliferation are known in the art and include, for example, MTT reduction and thymidine incorporation. A compound that reduces the level of proliferation compared to the level observed in the absence of the compound is considered to neutralize G-CSF signaling.

In a further example, the sCR1 conjugate that inhibits G-CSF signaling reduces mobilization of hematopoietic stem cells and/or endothelial progenitor cells in vivo following G-CSF administration and/or reduces the number of neutrophils in vivo, e.g., following G-CSF administration (however this is not essential). For example, the compound (e.g., a sCR1 variant conjugated to a protein that inhibits G-CSF signaling) is administered, optionally before, at the time of or after administration of G-CSF or a modified form thereof (e.g., PEGylated G-CSF or filgrastim). The number of hematopoietic stem cells (e.g., expressing CD34 and/or Thyl) and/or endothelial progenitor cells (e.g., expressing CD34 and VEGFR2) and/or neutrophils (identified morphologically and/or expressing e.g., CD10, CD14, CD31 and/or CD88) is assessed. A compound that reduces the level of the cell(s) compared to the level observed in the absence of the compound is considered to neutralize G-CSF signaling. In one example, the compound that inhibits G-CSF signaling reduces the number of neutrophils without inducing neutropenia.

### FXIIa Amidolytic Activity

Some conjugates encompassed by the present disclosure inhibit the amidolytic activity of human Factor XIIa. Methods of determining amidolytic activity of the conjugates of the disclosure will be apparent to the skilled person and/or described herein.

In one example, an in vitro assay is used to determine the level of FXIIa amidolytic activity. For example, the amidolytic activity can be measured by assay of the cleavage of FXII in the presence of conjugate of the disclosure and a buffer. For example, FXII is incubated in the presence of absence of a conjugate of the disclosure or control. Following incubation and addition of a detection substrate, the amidolytic activity is spectrophotometrically determined as a change in optical density (i.e., colour change). Proteins that are found to effectively inhibit amidolytic activity are identified as proteins that inhibit FXII activity.

### Determining Half Life

Some conjugates encompassed by the present disclosure have an improved half-life, e.g., are modified to extend their half-life compared to conjugates that are unmodified. Methods for determining a protein with an improved half-life will be apparent to the skilled person.

For example, the ability of a conjugate to bind to a neonatal Fc receptor (FcRn) is assessed. According to this method, the in vivo half-life of a conjugate of the disclosure can be measured in human FcRn transgenic mice (e.g., B6.Cg-Fcgrttm1Dcr Tg(FCGRT)32Dcr/DcrJ). For example, mice are intravenously injected with the conjugate and plasma collected at various time points. Blood is mixed with citrate buffer at a ratio of e.g., 8 parts blood 2 parts citrate buffer. Plasma levels of human sCR1 are measured in an anti-human CD35 ELISA. Mean residence time (MRT) and the area under the curve (AUC) are calculated using standard statistical formulae. In this regard, 20 increased binding affinity for FcRn increased the serum half-life of the molecule (see for example, Kim et al., Eur J Immunol., 24:2429, 1994).

The half-life of a conjugate of the disclosure can also be measured by pharmacokinetic studies, e.g., according to the method described by Kim et al, Eur J of Immunol 24:542, 1994. According to this method, radiolabeled protein is injected intravenously into mice and its plasma concentration is periodically measured as a function of time, for example at 3 minutes to 72 hours after the injection. The clearance curve thus obtained should be biphasic, that is, an alpha phase and beta phase. For the determination of the in vivo half-life of the protein, the clearance rate in beta-phase is calculated and compared with that of the wild type or unmodified protein.

### Pharmaceutical Compositions and Methods of Treatment

Suitably, in compositions or methods for administration, the sCR1 conjugate of the present disclosure (i.e., the sCR1 variant conjugated to a protein comprising an antigen binding domain) is combined with a pharmaceutically acceptable carrier as is understood in the art. Accordingly, one example of the present disclosure provides a composition (e.g., a pharmaceutical composition) comprising the sCR1 conjugate of the disclosure combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

A sCR1 conjugate of the present disclosure is useful for parenteral, topical, oral, or local administration, aerosol administration, intrathecal administration or transdermal administration, for prophylactic or for therapeutic treatment. In one example, the sCR1 conjugate is administered parenterally, such as subcutaneously or intravenously. For example, the sCR1 conjugate is administered intravenously.

Formulation of a sCR1 conjugate to be administered will vary according to the route of administration and formulation (e.g., solution, emulsion, capsule) selected. An appropriate pharmaceutical composition to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and any amino acid, including for example glycine or proline. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The composition can be stored in the liquid stage or can be lyophilized for storage and reconstituted in a suitable carrier prior to use according to art-known lyophilization and reconstitution techniques.

A method of the present disclosure may also include co-administration of the sCR1 conjugate according to the disclosure together with the administration of another therapeutically effective agent for inhibiting complement activity and/or G-CSF activity and/or antagonising activity of Factor XII and/or FactorXlla and/or antagonising activation of Factor XII and/or Factor XIIa or for the prevention or treatment of a complement-mediated disorder, a neutrophil-mediated disorder and/or a blood coagulation disorder.

In one example, the sCR1 conjugate of the disclosure is used in combination with at least one additional known compound or therapeutic protein which is currently being used or is in development for inhibiting complement activity or preventing or treating complement-mediated disorders. Compounds currently used in the treatment of complement-mediated disorders are known in the art, and include antibodies against C5 and activated forms thereof (C5a), e.g., eculizumab, Berinert Human C1 esterase inhibitor, Human C1 esterase inhibitor, Ruconest Recombinant C1 esterase inhibitor, Cinryze Human C1esterase inhibitor, Anti human MASP-2 monoclonal antibody, APL-2 C3-inhibiting peptide, Lampalizumab, TNT009 Anti-C1s Antibody. Additional compounds are described in Reis et al., Clin Immunol. Dec; 161(2): 225-240, 2015. Compounds currently used in the treatment of neutrophil-mediated disorders are known in the art, and include for example, an anti-inflammatory compound, immunomodulator or immunosuppressant, corticosteroid (e.g. a glucocorticoid, prednisone, prednisolone, beclometasone, budesonide, ciclesonide, or fluticasone), methotrexate, cyclophosphamide, a beta2 agonist (e.g., salbutamol, terbutaline sulfate, formoterol, vilanterol, or salmeterol), a leukotriene receptor antagonist (e.g., montelukast), muscarinic antagonists (e.g., ipratropium bromide), a theophylline (e.g., aminophylline), magnesium sulfate, a mast cell stabilizer (e.g., sodium cromoglycate or nedocromil), an anti-IL-5 antibody (e.g., mepolizumab), an anti-IgE antibody (e.g., omalizumab), an anti-1L-17A antibody (e.g., secukinumab), an anti-CD20 antibody (e.g., rituximab or ofatumumab), an anti-CD22 antibody (e.g., epratuzumab), an anti-TNF antibody (e.g., infliximab or adalimumab or golimumab) or soluble TNF receptor (e.g., etanercept), and a CTLA-4 antagonist (e.g., abatacept, CTLA4-Ig).

As will be apparent from the foregoing, the present disclosure provides methods of concomitant therapeutic treatment of a subject, comprising administering to a subject in need thereof an effective amount of a first agent and a second agent, wherein the first 30 agent is a sCRl conjugate of the present disclosure, and the second agent is also for inhibiting complement activity and/or G-CSF activity and/or antagonising activity of Factor XII and/or FactorXlla and/or antagonising activation of Factor XII and/or Factor XIIa or for the prevention or treatment of a complement-mediated disorder, a neutrophil-mediated disorder and/or a blood coagulation disorder.

As used herein, the term “concomitant” as in the phrase “concomitant therapeutic treatment” includes administering a first agent in the presence of a second agent. A concomitant therapeutic treatment method includes methods in which the first, second, third or additional agents are co-administered. A concomitant therapeutic treatment method also includes methods in which the first or additional agents are administered in the presence of a second or additional agent, wherein the second or additional agent, for example, may have been previously administered. A concomitant therapeutic treatment may be executed step-wise by different actors. For example, one actor may administer to a subject a first agent and as a second actor may administer to the subject a second agent and the administering steps may be executed at the same time, or nearly the same time, or at distant times, so long as the first agent (and/or additional agents) are after administration in the presence of the second agent (and/or additional agents). The actor and the subject may be the same entity (e.g. a human)

The optimum concentration of the active ingredient(s) in the chosen medium can be determined empirically, according to procedures known to the skilled artisan, and will depend on the ultimate pharmaceutical formulation desired.

The dosage ranges for the administration of the sCR1 conjugate of the disclosure are those large enough to produce the desired effect. For example, the composition comprises an effective amount of the sCR1 conjugate. In one example, the composition comprises a therapeutically effective amount of the sCR1 conjugate. In another example, the composition comprises a prophylactically effective amount of the sCR1 conjugate.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the sCR1 conjugate is administered at an initial (or loading) dose which is higher than subsequent (maintenance doses). For example, the sCR1 conjugate is administered at an initial dose of between about 10 mg/kg to about 30 mg/kg. The sCR1 conjugate is then administered at a maintenance dose of between about 0.0001 mg/kg to about 30 mg/kg. The maintenance doses may be administered every 2-30 days, such as, every 2 or 3 or 6 or 9 or 12 or 15 or 18 or 21 or 24 or 27 or 30 days.

In some examples, a dose escalation regime is used, in which a sCR1 conjugate is initially administered at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

A subject may be retreated with the sCR1 conjugate, by being given more than one exposure or set of doses, such as at least about two exposures, for example, from about 2 to 60 exposures, and more particularly about 2 to 40 exposures, most particularly, about 2 to 20 exposures.

In one example, any retreatment may be given when signs or symptoms of disease return, e.g., a bacterial infection.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

Administration of a sCR1 conjugate according to the methods of the present disclosure can be continuous or intermittent, depending, for example, on the recipient's physiological condition, whether the purpose of the administration is therapeutic or prophylactic, and other factors known to skilled practitioners. The administration may be essentially continuous over a preselected period of time or may be in a series of spaced doses, e.g., either during or after development of a condition.

### Kits and Other Compositions of Matter

Another example of the disclosure provides kits containing a sCR1 conjugate of the present disclosure useful for inhibiting complement activity and/or G-CSF activity or for the treatment or prevention of a complement-mediated disorder, a neutrophil-mediated disorder and/or a blood coagulation disorder as described above.

Another example of the disclosure provides kits containing a sCR1 conjugate of the present disclosure useful for inhibiting complement activity and/or antagonising activity of Factor XII and/or Factor XIIa and/or Factor XI and/or Factor XIa and/or antagonising activation of Factor XII and/or Factor XIIa and/or Factor XI and/or Factor Xia or for the treatment or prevention of a complement-mediated disorder, a neutrophil-mediated disorder and/or a blood coagulation disorder as described above.

In one example, the kit comprises (a) a container comprising a sCR1 conjugate optionally in a pharmaceutically acceptable carrier or diluent; and (b) a package insert with instructions for inhibiting complement activity and/or G-CSF activity or for treating or preventing a complement-mediated disorder and/or a neutrophil-mediated disorder in a subject.

In one example, the kit comprises (a) at least one sCR1 conjugate optionally in a pharmaceutically acceptable carrier or diluent; (b) instructions for using the kit in inhibiting complement activity and/or G-CSF activity or for treating or preventing a complement-mediated disorder and/or neutrophil-mediated disorder in the subject; and (c) optionally, at least one further therapeutically active compound or drug.

In one example, the kit comprises (a) a container comprising a sCR1 conjugate optionally in a pharmaceutically acceptable carrier or diluent; and (b) a package insert with instructions for inhibiting complement activity and/or antagonising activity of Factor XII and/or Factor XIIa and/or antagonising activation of Factor XII and/or Factor XIIa or for treating or preventing a complement-mediated disorder, a neutrophil-mediated disorder and/or a blood coagulation disorder in a subject.

In one example, the kit comprises (a) a container comprising a sCR1 conjugate optionally in a pharmaceutically acceptable carrier or diluent; and (b) a package insert with instructions for inhibiting complement activity and/or antagonising activity of Factor XI and/or Factor XIa and/or antagonising activation of Factor XI and/or Factor XIa or for treating or preventing a complement-mediated disorder, a neutrophil-mediated disorder and/or a blood coagulation disorder in a subject.

In one example, the kit comprises (a) at least one sCR1 conjugate optionally in a pharmaceutically acceptable carrier or diluent; (b) instructions for using the kit in inhibiting complement activity and/or antagonising activity of Factor XII and/or Factor XIIa and/or antagonising activation of Factor XII and/or Factor XIIa or for treating or preventing a complement-mediated disorder, a neutrophil-mediated disorder and/or a blood coagulation disorder in a subject; and (c) optionally, at least one further therapeutically active compound or drug.

In one example, the kit comprises (a) at least one sCR1 conjugate optionally in a pharmaceutically acceptable carrier or diluent; (b) instructions for using the kit in inhibiting complement activity and/or antagonising activity of Factor XI and/or Factor XIa and/or antagonising activation of Factor XI and/or Factor XIa or for treating or preventing a complement-mediated disorder, a neutrophil-mediated disorder and/or a blood coagulation disorder in a subject; and (c) optionally, at least one further therapeutically active compound or drug.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for inhibiting complement activity and/or G-CSF activity and/or antagonising activity of Factor XII and/or Factor XIIa and/or antagonising activation of Factor XII and/or Factor XIIa and/or antagonising activity of Factor XI and/or Factor XIa and/or antagonising activation of Factor XI and/or Factor XIa or for treating or preventing a complement-mediated disorder, a neutrophil-mediated disorder and/or a blood coagulation disorder and may have a sterile access port (for example, the container may be an intravenous solution bag or a vial having a stopper pierceable by a hypodermic injection needle). At least one active agent in the composition is the sCR1 conjugate. The label or package insert indicates that the composition is used for treating a subject eligible for treatment, e.g., one having or predisposed to developing a complement-mediated disorder and/or a neutrophil-mediated disorder, with specific guidance regarding dosing amounts and intervals of the sCR1 conjugate and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The kit optionally further comprises a container comprising a second medicament, wherein the sCR1 conjugate is a first medicament, and which article further comprises instructions on the package insert for treating the subject with the second medicament, in an effective amount. The second medicament may be a therapeutic protein set forth above.

The present disclosure includes the following non-limiting Examples.

### EXAMPLES

### Example 1

### Generation of sCR1 Variants

Human Complement Receptor Type 1 (CR1) cDNA (GenBank Accession no. NP_000564) was codon-optimized for human expression and synthesized by Geneart® (Invitrogen™, Thermo Fisher Scientific). Full-length and truncated soluble CR1 (sCR1) variants were generated using standard PCR-based mutagenesis techniques. cDNA was generated with a Kozak consensus sequence (GCCACC) immediately upstream of the initiating methionine (+1), following which it was digested with NheI and XhoI and ligated into pcDNA3.1 (Invitrogen™, Thermo Fisher Scientific). sCR1 variant cDNA was cloned in-frame with a C-terminal 8× Histidine-tag. See Table 1 for a list of sCR1-8His variants.

Large-scale preparations of plasmid DNA were carried out using QIAGEN Plasmid Giga Kits according to the manufacturer's instructions. The nucleotide sequences of all plasmid constructs were verified by sequencing both strands using BigDye™Terminator Version 3.1 Ready Reaction Cycle Sequencing (Invitrogen™ Thermo Fisher Scientific) and an Applied Biosystems 3130×1 Genetic Analyzer.

Transient transfections of Expi293F™ cells with sCR1 expression plasmids were performed using the Expi293™ Expression system according to the manufacturer's recommendations (Invitrogen™, Thermo Fisher Scientific). All cell culture media were supplemented with Antibiotic-Antimycotic (GIBCO®, Thermo Fisher Scientific) and cells were maintained at 37° C. in incubators with an atmosphere of 8% CO₂.

sCR1-8His polypeptides were purified. Briefly, for purification of hexahistidine tagged sCR1 proteins, the culture supernatant was loaded directly onto nickel sepharose excel affinity resin (GE Healthcare) pre-equilibrated with 20mM NaH₂PO₄, 500 mM NaCl, 10 mM Imidazole, pH 7.4. After loading, the resin was washed with 20 mM NaH₂PO₄, 500 mM NaCl, 25 mM Imidazole, pH 7.4. Resin-bounded sCR1 was block eluted with 20 mM NaH₂PO₄, 500mM NaCl, 500 mM Imidazole, pH 7.4 collecting eluted protein based on absorbance at 280 nm. Collected protein was loaded onto a HiLoad 26/60 superdex200 prep grade column (GE Healthcare) pre-equilibrated in mt-PBS (137mM NaCl, 27 mM KCl, 8.1 mM Na₂HPO₄, 1.15 mM KH₂PO₄, pH 7.4) to remove any contaminating proteins and buffer exchange into desired buffer. Purified protein was concentrated using amicon ultra centrifugal filters with 50 kDa MWCO to desired concentration, sterile filtered and stored at −80° C. Due to intracellular processing, the mature sCR1-8His variants lack the N-terminal 41 aa human CR1 signal peptide.

TABLE 1
sCR1-8His variants

| | | Length of mature | |
|---|---|---|---|
| | | sCR1 variant | |
| | | protein (aa's) - | |
| | | (less 41 aa human | |
| | | CR1 signal | |
| | LHR | peptide and less | |
| Identifier | regions | 8His tag) | SEQ ID NO: |
| sCR1(1971)-8His | ABCD | 1930 | Signal peptide: SEQ ID NO: 18 |
| SEQ ID NO: 20 | | | sCR1 sequence: SEQ ID NO: 2 |
| | | | 8xHis-tag: SEQ ID NO: 17 |
| sCR1(1392)-8His | ABC | 1351 | Signal peptide: SEQ ID NO: 18 |
| SEQ ID NO: 21 | | | sCR1 sequence: SEQ ID NO: 3 |
| | | | 8xHis-tag: SEQ ID NO: 17 |
| sCR1(939)-8His | AB | 898 | Signal peptide: SEQ ID NO: 18 |
| SEQ ID NO: 22 | | | sCR1 sequence: SEQ ID NO: 4 |
| | | | 8xHis-tag: SEQ ID NO: 17 |
| sCR1(490-1392)-8His | BC | 903 | Signal peptide: SEQ ID NO: 19 |
| SEQ ID NO: 23 | | | sCR1 sequence: SEQ ID NO: 5 |
| | | | 8xHis-tag: SEQ ID NO: 17 |
| sCR1(490-1971)-8His | BCD | 1482 | Signal peptide: SEQ ID NO: 19 |
| SEQ ID NO: 24 | | | sCR1 sequence: SEQ ID NO: 6 |
| | | | 8xHis-tag: SEQ ID NO: 17 |
| sCR1(234)-8His | A′ | 193 | Signal peptide: SEQ ID NO: 18 |
| SEQ ID NO: 25 | | | sCR1 sequence: SEQ ID NO: 7 |
| | | | 8xHis-tag: SEQ ID NO: 17 |
| sCR1(489)-8His | A | 448 | Signal peptide: SEQ ID NO: 18 |
| SEQ ID NO: 26 | | | sCR1 sequence: SEQ ID NO: 8 |
| | | | 8xHis-tag: SEQ ID NO: 17 |
| sCR1(940-1971)-8His | CD | 1032 | Signal peptide: SEQ ID NO: 19 |
| SEQ ID NO: 27 | | | sCR1 sequence: SEQ ID NO: 9 |
| | | | 8xHis-tag: SEQ ID NO: 17 |
| sCR1(490-939)-8His | B | 450 | Signal peptide: SEQ ID NO: 19 |
| SEQ ID NO: 28 | | | sCR1 sequence: SEQ ID NO: 10 |
| | | | 8xHis-tag: SEQ ID NO: 17 |
| sCR1(940-1392)-8His | C | 453 | Signal peptide: SEQ ID NO: 19 |
| SEQ ID NO: 29 | | | sCR1 sequence: SEQ ID NO: 11 |
| | | | 8xHis-tag: SEQ ID NO: 17 |
| sCR1(1393-1971)-8His | D | 579 | Signal peptide: SEQ ID NO: 19 |
| SEQ ID NO: 30 | | | sCR1 sequence: SEQ ID NO: 12 |
| | | | 8xHis-tag: SEQ ID NO: 17 |

### Example 2

### sCR1-8His Variants have Complement Inhibitory Activity In Vitro

To assess complement inhibitory activity, the sCR1-8His variants were tested in the Wieslab® complement assay (Euro Diagnostica) according to manufacturer's instructions. Briefly, sCR1-8His variant proteins were serially diluted in PBS in a 96-well plate. 50 μl of each diluted sCR1-8His variant sample or PBS alone was added to 202.5 μl of pre-diluted human serum (1:101 for classical/lectin) or 220 μl of diluted serum (1:18 for alternative) in the appropriate assay diluent for each complement pathway (as per manufacturer's instructions) and incubated for 30 min at room temperature (RT). Once added to the pre-diluted serum, the final starting concentration of each protein was 40 nM. 100 μl of each sample was transferred to the assay plate in duplicate and incubated for 1 hr at 37° C. (with no CO₂). Wells were emptied and washed three times with 300 μl/well of 1× wash buffer (as per manufacturer's instructions). The terminal complex of C5b-9 was detected using 100 μl/well alkaline-phosphatase conjugated anti-C5b-9 specific monoclonal antibody, which was incubated for 30 min at RT. Unbound antibody was discarded and wells were washed three times with 300 μl/well of 1× wash buffer. Bound antibodies were detected using 100 μl/well alkaline phosphatase substrate solution and incubated for 30 min at RT. Absorbance at 405 nm was read using the Envision plate reader.

Raw values were expressed as a percentage of C5b-9 formation by the serum and PBS only control (i.e. 100% C5b-9 formation). Results were analysed in Graph Pad Prism for IC₅₀ values using a log(inhibitor) vs. response—Variable slope (four parameters) fit. Bottom and top constrained to values 0 and 100, respectively.

All sCR1-8His variants except sCR1(490-939)-8His, sCR1(940-1392)-8His and sCR1(1393-1971)-8His had functional activity in the classical, lectin and alternative 25 pathways. sCR1(490-939)-8His had functional activity in the alternative pathway only, whilst sCR1(940-1392)-8His had functional activity in the lectin and alternative pathways. sCR1(1939-1971)-8His had no detectable activity in any of the classical, lectin or alternative pathways.

As shown in Table 2 below, sCR1(1392)-8His had increased inhibitory activity in all three complement pathways (i.e., classical, lectin and alternative) compared to full-length sCR1(1971)-8His and other sCR1 fragments in the Wieslab assays.

sCR1-8His variants were also tested for functional activity using a hemolysis assay (e.g., classical pathway (i.e., CH50) and alternative pathway (ApH50) inhibition assays).

To assess the inhibition of the classical pathway of the complement system (i.e., CH50) by sCR1 variants, sheep erythrocytes (Siemens) were sensitized with rabbit anti-sheep antibodies (Ambozeptor 6000; Siemens) and diluted to 4×10⁸ cells/mL GVB⁺⁺ (GVB, 0.15 mM CaCl₂, 0.5 mM MgCl₂). sCR1 variants were pre-incubated in 1/40 diluted NHS (30 min at RT) and subsequently added to the erythrocytes at a 1/1 (v/v) ratio and incubated during 1 h at 37° C. in a microtiter-plate shaking device. After adding ice-cold GVBE (GVB, 10 mM EDTA) and centrifugation (5 min at 125033 g, 4° C.), hemolysis was determined in the supernatant by measuring the absorbance of released hemoglobin at 412 nm. Cells incubated with NHS and buffer only served as 100% lysis controls. The inhibition of lysis by the sCR1 variants was calculated relative to control.

To assess the inhibition of the alternative pathway of the complement system (i.e., ApH50) by sCR1 variants, rabbit erythrocytes (Jackson Laboratories) were washed and diluted to 2×10⁸ cells/mL GVB/MgEGTA (GVB, 5 mM MgEGTA). sCR1 variants were pre-incubated in ⅙ diluted NHS (30 min at RT) and subsequently added to the erythrocytes at a 2/1 (v/v) ratio and incubated during 1 h at 37° C. in a microtiter-plate shaking device. After adding ice-cold GVBE and centrifugation (10 min at 1250×g), hemolysis was determined in the supernatant by measuring the absorbance of released hemoglobin at 412 nm. Cells incubated with NHS and buffer only served as 100% lysis controls. The inhibition of lysis by the sCR1 variants was calculated relative to control.

All variants except sCR1(1393-1971)-8His displayed functional activity in both the CH50 and ApH50 assays. As shown in Table 3 sCR1(1392)-8His had increased activity compared to sCR1(1971)-8His in both assays.

TABLE 2
Relative in vitro activity of sCR1 variants in Wieslab assays

| | Wieslab Assay | | | |
|---|---|---|---|---|
| | Exp't | Classical | Lectin | Alternative |
| sCR1 variant | no. | IC₅₀ (nM) | IC₅₀ (nM) | IC₅₀ (nM) |
| sCR1(1971)-8His | 1 | 1.50 | 1.20 | 0.821 |
| [ABCD] | 2 | 1.18 | 0.66 | 0.876 |
| | 3 | 1.40 | 0.78 | — |
| sCR1(1392)-8His | 1 | 0.879 | 0.547 | 0.272 |
| [ABC] | 2 | 0.402 | 0.428 | 0.384 |
| | 3 | 0.583 | 0.458 | — |
| sCR1(939)-8His | 1 | 4.04 | 2.20 | 3.41 |
| [AB] | 2 | 1.60 | 1.01 | 2.72 |
| sCR1(490-1971) | 1 | 7.02 | 3.80 | 0.295 |
| [BCD] | 2 | 2.90 | 4.29 | 1.31 |
| sCR1(490-1392) | 1 | 12.95 | 5.08 | 0.579 |
| [BC] | 2 | 5.70 | 2.33 | 1.33 |
| sCR1(1-234) [A′] | | 23.68 | 15.4 | 1.31 |
| sCR1(1-489) [A] | | 45.26 | 27.8 | 2.73 |
| sCR1(940-1971) [CD] | | 180.4 | 121.6 | 2.58 |
| CR1(490-939) [B] | | No Activity | ND | 3.21 |
| CR1(940-1392) [C] | | No Activity | 898.2 | 1.45 |
| CR1(1393-1971) [D] | | No Activity | No Activity | No Activity |

TABLE 3
Relative in vitro activity of sCR1 variants in hemolysis assays

Hemolysis Assay

| | Classical | Alternative |
|---|---|---|
| sCR1 variant | IC₅₀ (pM) | IC₅₀ (pM) |
| sCR1(1971)-8His [ABCD] | 1.03 | 2.11 |
| sCR1(1392)-8His [ABC] | 0.427 | 0.956 |
| sCR1(939)-8His [AB] | 4.44 | 13.35 |
| sCR1(490-1971) [BCD] | 32.05 | 45.46 |
| sCR1(490-1392) [BC] | 9.69 | 13.88 |
| sCR1(1-234) [A′] | 35.7 | 45.09 |
| sCR1(1-489) [A] | 74.95 | 51.59 |
| sCR1(940-1971) [CD] | 658.5 | 90.49 |
| sCR1(490-939) [B] | 948.5 | 64.38 |
| sCR1(940-1392) [C] | 716.4 | 63.62 |
| sCR1(1393-1971) [D] | No Activity | No Activity |

### Example 3

### sCR1(1392)-8His Variant Shows Increased Stability Compared to sCR1(1971)-8His

To assess the stability of sCR1(1392)-8His in different buffer conditions, a differential scanning fluorimetry (DSF) assay was performed to measure the thermal stability of the sCR1(1392)-8His protein compared to the full length sCR1(1971)-8His protein. The stability of the proteins was assessed under a range of salt (NaCl 0 mM, 50 mM, 150 mM and 500 mM) and pH conditions for the following buffers: citrate, HEPES, sodium acetate, phosphate, glycine, histidine, TRIS and proline.

Briefly, 5μl of 4× buffer concentrate were dispensed in duplicate in a 384-well plate. sCR1(1392)-8His and sCR1(1971)-8His proteins were diluted to 0.13 mg/ml in MT-PBS then spiked with a 1/20 dye stock (Sypro® Orange; Sigma) made up in water to give a 1/400 final dilution in each assay reaction. 15 μ of protein/dye mixture were then dispensed into each well of the 384-well plate containing the buffer concentrate. The plate was sealed with an optical adhesive cover and centrifuged for 1 minute at 3220 g prior to running on the QuantStudio™ Real-Time PCR instrument (Applied Biosystems). A melt curve was generated by cooling and holding the temperature for 1 minute at 20.0° C., before ramping up from 20.0° C. to 99.0° C. at a rate of 0.05° C./s. Protein Thermal Shift software (Applied Biosystems) was used to calculate the transition midpoint (Tₘ) values from each melting curve using the first derivative function. Contour plots were generated using JMP13 to graphically display how the Tₘ values change in relation to NaCl concentration (x axis) and pH (y axis).

sCR1(1392)-8His was stable under several buffer conditions including: phosphate (pH6.0-8.0; NaCl 0-500 mM); phosphate-citrate (pH6.0-8.0; NaCl 0-500 mM); Tris (pH7.0-9.0; NaCl 0-500mM); glycine (pH9.0-10.0; NaCl 0-500 mM); HEPES (pH6.5-8.5; NaCl 0-500 mM) and histidine (pH6.0-7.0; NaC 0-500 mM). The maximum Tₘ value measured was 61.4° C. for sCR1(1392)-8His and 61.7° C. for sCR1(1971)-8His.

Based on the buffer screen, sCR1(1392)-8His was more stable than sCR1(1971)-8His.

### Example 4: Sialylated sCR1(1392)-8His has Improved in Vivo Half-Life

To assess whether the in vivo half-life of sCR1(1392)-8His could be extended, a sialylated version of sCR1(1392)-8His was prepared (sCR1(1392)-8His^{SIA}). Briefly, the sialylated material was generated by co-transfecting Expi293F cells with the cDNA encoding sCR1(1392)-8His together with the cDNA encoding human ST3GAL3 (ST3 beta-galactoside alpha-2,3-sialyltransferase 3, GenBank Accession no. NP_006270) and the cDNA encoding human B4GALT1 (human β1,4-galactosyltransferase, GenBank Accession no. NP_001488.2) at a 94:3:3 ratio.

As shown in Table 4, sCR1(1392)-8His^{SIA} material produced in ST3GAL3/B4GALT1-transfected cells had a much higher proportion of sialylated glycans. In particular, sialylated sCR1(1392)-8His^{SIA} material had a higher proportion of di-, tri- and tetra-sialylated glycans.

TABLE 4
Proportion of glycans in sialylated sCR1(1392)-8His

| | | sCR1(1392)- | sCR1(1392)- |
|---|---|---|---|
| | | 8His | 8His^{SIA} |
| Peak | | % of Total | % of Total |
| No. | Glycan Group | Peak Area | Peak Area |
| 1 | Asialylated | 74.5 | 24.1 |
| 2 | Monosialylated | 21.1 | 22.8 |
| 3 | Disialylated | 3.7 | 41.9 |
| 4 | Trisialylated | 0.6 | 9.1 |
| 5 | Tetrasialylated | 0.1 | 2.1 |

The in vivo half-life of sCR1(1392)-8His and the sialylated version thereof (sCR1(1392)-8His^{SIA}) was tested in human FcRn transgenic mice (B6.Cg-Fcgrt^{tm1Dcr} Tg(FCGRT)32Dcr/DcrJ; The Jackson Laboratory stock number 014565). Mice were intravenously injected with 30 mg/kg of sCR1(1392)-8His or sCR1(1392)-8His^{SIA} and plasma collected at various time points (Group A: 5 min and 4 h, n=3; Group B: 0.5 h and 8 h, n=3; Group C: 1 h and 16 h, n=3; Group D: 2 h and 48 h, n=3). Blood was mixed with citrate buffer at a ratio of 8 parts blood 2 parts citrate buffer. Plasma levels of human sCR1 were measured in an anti-human CD35 ELISA (RayBiotech, cat no. ELH CD35) according to manufacturer's instructions, with the following modifications: standard curves (ranging from 3-250 ng/mL) were generated using each test article, the assay buffer used was 1% BSA heat shock fraction, protease free (Sigma cat no. A3059), and the wash buffer was PBS+0.05% v/v Tween-20. Mean residence time (MRT) and the area under the curve (AUC) were calculated using standard statistical formulae.

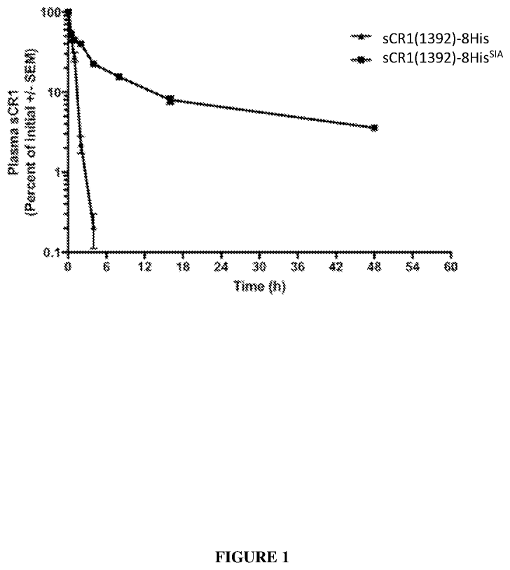

As shown in FIG. 1, the sCR1(1392)-8His^{SIA} had improved in vivo retention compared to sCR1(1392)-8His, with a 25-fold increased MRT (14.7 hours vs 35 mins) and an 8-fold increase in the AUC (AUC=516.5 vs 65.74).

### Example 5

### Generation of sCR1 Variant—Anti-G-CSFR Conjugates

Recombinant sCR1 fusions were generated by fusing the sCR1(1392) variant with scFv's of a mouse anti-G-CSFR antibody VR81 or scFv's of antibody C1.2 at the C-terminus of the sCR1 sequence (Table 5).

VR81 is a mouse monoclonal IgG1κ antibody produced against the extracellular domain of murine G-CSFR and blocks G-CSF binding to G-CSFR as described (Campbell et al. Journal of Immunology, 197(11) (2016) 4392-4402). In this regard, VR81 is a mouse surrogate antibody for C1.2, which is described in WO2012171057.

Recombinant fusions were made using standard cloning techniques. A GS16 linker was used to link the sCR1 sequence and the anti-G-CSFR sequence. A signal peptide was also employed. All fusion proteins were expressed in Expi293F™ cells and sCR1 proteins purified as described above.

TABLE 5
sCR1(1392)-anti-G-CSFR fusions

| Identifier | SEQ ID NO: |
|---|---|
| sCR1(1392)-GS16- | Signal peptide: SEQ ID NO: 18 |
| C1.2scFvLH | sCR1(1392) sequence: SEQ ID NO: 3 |
| SEQ ID NO: 49 | GS16 Linker: SEQ ID NO: 33 |
| | C1.2 VH sequence: SEQ ID NO: 36 |
| | C1.2 VL sequence: SEQ ID NO: 37 |
| | GS15 Linker: SEQ ID NO: 32 |
| sCR1(1392)-GS16- | Signal peptide: SEQ ID NO: 18 |
| C1.2scFvHL | sCR1(1392) sequence: SEQ ID NO: 3 |
| SEQ ID NO: 50 | GS16 Linker: SEQ ID NO: 33 |
| | C1.2 VH sequence: SEQ ID NO: 36 |
| | C1.2 VL sequence: SEQ ID NO: 37 |
| | GS15 Linker: SEQ ID NO: 32 |
| sCR1(1392)-GS16- | Signal peptide: SEQ ID NO: 18 |
| 5E2VR81scFvLH | sCR1(1392) sequence: SEQ ID NO: 3 |
| SEQ ID NO: 51 | GS16 Linker: SEQ ID NO: 33 |
| | 5E2VR81 V_{H} sequence: SEQ ID NO: 46 |
| | 5E2VR81 V_{L} sequence: SEQ ID NO: 47 |
| | GS20 Linker: SEQ ID NO: 34 |
| sCR1(1392)-GS16- | Signal peptide: SEQ ID NO: 18 |
| 5E2VR81scFvHL | sCR1(1392) sequence: SEQ ID NO: 3 |
| SEQ ID NO: 52 | GS16 Linker: SEQ ID NO: 33 |
| | 5E2VR81 V_{H} sequence: SEQ ID NO: 46 |
| | 5E2VR81 VL sequence: SEQ ID NO: 47 |
| | GS20 Linker: SEQ ID NO: 34 |

High level expression of a 174 kDa expression product and a 175 kDa expression product was observed for each sCR1(1392)-C1.2scFv fusion and each sCR1(1392)-5E2VR81scFv fusion, respectively (data not shown). The expression products were soluble, expressed at a high level and at the expected size.

To assess complement inhibitory activity, the sCR1-anti-G-CSFR conjugates were tested in the Wieslab® complement assay (Euro Diagnostica) according to manufacturer's instructions and as described above. As shown in Table 6 below, all sCR1-anti-G-CSFR conjugates (i.e., both orientations) had increased functional activity in the classical, lectin and alternative pathways compared to the unconjugated 15 sCR1(1392) variant.

TABLE 6
Relative in vitro activity of sCR1 variants in Wieslab assays

| | Wieslab Assay | | |
|---|---|---|---|
| | Classical | Lectin | Alternative |
| sCR1 variant | IC₅₀ (nM) | IC₅₀ (nM) | IC₅₀ (pM) |
| sCR1(1392) | 1.34 | 0.85 | 667.4 |
| sCR1(1392)-GS16- | 0.78 | 0.53 | 367.9 |
| 5E2VR81scFvLH | | | |
| sCR1(1392)-GS16- | 0.63 | 0.55 | 479.1 |
| 5E2VR81scFvHL | | | |

### Example 6

### sCR1 variant—Anti-G-CSFR Conjugates have G-CSF Signalling Inhibitory Activity In Vitro

G-CSF signalling inhibitory activity of the sCR1-5E2VR81 anti-G-CSFR conjugates, was assessed in vitro (as previously described in Campbell et al., J. Immunol. 2016) by determining their ability to inhibit murine G-CSF (mG-CSF)-dependent proliferation of a G-CSF dependent cell line (mouse NFS-60).

Firstly, a dose response of the mouse NFS-60 cells to mG-CSF was determined. Briefly, cells were plated into a 96 well plate (1×10⁴ cells per well) and titrating concentrations of mG-CSF (100 ng/ml, ⅓ dilution, 12 points) and mIL-3 were added to cells and left to incubate for 48 hours, 37° C., 5% CO₂. After incubation, cells were analyzed using a Vialight kit, following manufacturer's instructions, in order to measure cell viability. An EC₅₀ of 0.05343ng/ml and 0.1431 ng/ml was determined for mG-CSF and mIL-3 respectively.

To assess relative inhibition of m-G-CSF by the sCR1-anti-G-CSFR conjugates and the mouse VR81 antibody, mouse NFS-60 cells were plated into a 96 well plate (1×10⁴ cells per well) and titrating concentrations of VR81, sCR1(1392)-GS16-5E2VR81scFvLH, and sCR1(1392)-GS16-5E2VR81scFvHL was added to corresponding wells (100 ug/ml, ⅓ dilution, 16 points). Fixed concentrations of either 1 ng/ml or 0.1 ng/ml of mG-CSF was added to corresponding wells and plates were incubated for 48 hours, 37° C., 5% CO₂. After incubation, cells were analyzed using a Vialight kit, following manufacturer's instructions, in order to measure cell viability.

As shown in Table 7 below, the sCR1-anti-G-CSFR conjugates showed a similar effect when cells are stimulated with 0.1 ng/ml G-CSF or 1 ng/ml G-CSF. Both sCR1-5E2VR81scFv conjugates (LH and HL orientations) had similar potency in the G-CSF inhibition assays although the conjugates were approximately 500× less potent compared to the VR81 antibody.

TABLE 7
In vitro inhibitory activity of the sCR1-anti-G-CSFR conjugates

IC₅₀ (nM)

| | Stimulated with | Stimulated with |
|---|---|---|
| | 1.0 ng/ml | 0.1 ng/ml |
| sCR1 variant | mG-CSF | mG-CSF |
| VR81 | 3.23 × 10⁻³ | 1.04 × 10⁻³ |
| sCR1(1392)-GS16- | 1.89 | 5.20 × 10⁻¹ |
| 5E2VR81scFvLH | | |
| sCR1(1392)-GS16- | 1.49 | 5.18 × 10⁻¹ |
| 5E2VR81scFvHL | | |

### Example 7

### Generation of sCR1 Variant—Anti-Factor XII scFv Conjugates

Recombinant sCR1 fusions were generated by fusing sCR1(1392) variant with an scFv of antibody 3F7 or 3F7G at the C-terminus of the sCR1 sequence (Table 8).

TABLE 8
sCR1(1392)-anti-Factor XII scFv fusions

| Identifier | SEQ ID NO: |
|---|---|
| sCR1(1392)-GS16- | Signal peptide: SEQ ID NO: 18 |
| 3F7scFvHL | sCR1(1392) sequence: SEQ ID NO: 3 |
| SEQ ID NO: 43 | GS16 Linker: SEQ ID NO: 33 |
| | 3F7 VH sequence: SEQ ID NO: 56 |
| | 3F7 VL sequence: SEQ ID NO: 57 |
| | GS16 Linker: SEQ ID NO: 55 |
| sCR1(1392)-GS16- | Signal peptide: SEQ ID NO: 18 |
| 3F7GscFvHL | sCR1(1392) sequence: SEQ ID NO: 3 |
| SEQ ID NO: 44 | GS16 Linker: SEQ ID NO: 33 |
| | 3F7G VH sequence: SEQ ID NO: 58 |
| | 3F7G V_{L} sequence: SEQ ID NO: 59 |
| | GS16 Linker: SEQ ID NO: 55 |

High level expression of a 175 kDa expression product was observed (data not shown). The expression products were soluble, expressed at a high level and at the expected size.

### Example 8

### sCR1 Variant—Anti-Factor XII scFv Conjugates have FXIIa Inhibitory Activity In Vitro

To assess inhibitory activity of the sCR1-anti-Factor XII scFv conjugates generated above, a chromogenic inhibitory assay was performed.

Briefly, 20 μL Factor XIIa (1 μg) was mixed with buffer containing either the sCR1(1392)-GS16-3F7scFvHL, sCR1(1392)-GS16-3F7GscFvHL or unconjugated 3F7 (ch3F7-mG1L-aFXII) to a volume of 160 μL in an ELISA plate. After incubation for 5 min at 37° C., 40 μL of the chromogenic substrate were added. The mixture was incubated for 15 min at 37° C. before 40 μL of the stop solution was added. The measurement was performed in a plate reader at 405 nm.

As shown in Table 9 below, all sCR1-anti-Factor XII scFv conjugates had functional FXIIa inhibitory activity similar to that of the 3F7 antibody.

TABLE 9
Relative in vitro activity of sCR1 variants in Chromogenic Assay

| Protein | IC₅₀ (nM) |
|---|---|
| ch3F7-mGlL-aFXII (3F7) | 17.19 |
| sCR1(1392)-GS16-3F7GscFvHL | 27.74 |
| sCR1(1392)-GS16-3F7scFvHL | 23.71 |

### Example 9

### sCR1 variant—Anti-Factor XII scFv Conjugates have Complement Inhibitory Activity in Vitro

To assess complement inhibitory activity, the sCR1-anti-FXII conjugates were tested in the Wieslab® complement assay (Euro Diagnostica) according to manufacturer's instructions and as described above. As shown in Table 10 below, all sCR1-anti-FXII conjugates (i.e., both orientations) had increased functional activity in the classical, lectin and alternative pathways compared to the unconjugated sCR1(1392) variant.

TABLE 10
Relative in vitro activity of sCR1 variants in Wieslab assays

| | Wieslab Assay | | |
|---|---|---|---|
| | Classical | Lectin | Alternative |
| sCR1 variant | IC₅₀ (nM) | IC₅₀ (nM) | IC₅₀ (pM) |
| sCR1(1392) | 1.06 | 0.70 | 116 |
| sCR1(1392)-GS16- | 0.63 | 0.46 | 50.22 |
| 3F7scFvHL | | | |
| sCR1(1392)-GS16- | 0.48 | 0.37 | 53.55 |
| 3F7GscFvHL | | | |

1. A soluble complement receptor type 1 (sCR1) conjugate comprising:
(i) a sCR1 variant comprising an amino acid sequence selected from the group consisting of:
a) an amino acid sequence corresponding to amino acids 42 to 939 of SEQ ID NO: 1;
b) an amino acid sequence corresponding to amino acids 490 to 1392 of SEQ ID NO: 1; and
(ii) a protein comprising an antigen binding domain that binds to a target and inhibits signaling by or via the target.

2. The sCR1 conjugate of claim 1, wherein the sCR1 variant comprises:
(i) an amino acid sequence corresponding to amino acids 42 to 1392 of SEQ ID NO: 1;
(ii) an amino acid sequence corresponding to amino acids 42 to 939 of SEQ ID NO: 1;
(iii) an amino acid sequence corresponding to amino acids 490 to 1392 of SEQ ID NO: 1; or
(iv) an amino acid sequence corresponding to amino acids 490 to 1971 of SEQ ID NO: 1.

3. The sCR1 conjugate of claim 1 or 2, wherein the sCR1 variant comprises an amino acid sequence corresponding to amino acids 42 to 1392 of SEQ ID NO: 1.

4. The sCR1 conjugate according to any one of claims 1 to 3, wherein the sCR1 variant has increased complement inhibitory activity compared to a sCR1 comprising a sequence set forth in SEQ ID NO: 2.

5. The sCR1 conjugate according to any one of claims 1 to 4, wherein the sCR1 variant has increased complement inhibitory activity in the classical pathway, the lectin pathway and/or alternative complement pathway compared to a sCR1 comprising a sequence set forth in SEQ ID NO: 2.

6. The sCR1 conjugate according to any one of claims 1 to 5, wherein the sCR1 variant comprises long homologous repeat (LHR) regions selected from the group consisting of:
(i) LHR-A and LHR-B;
(ii) LHR-A, LHR-B and LHR-C;
(iii) LHR-B and LHR-C; and
(iv) LHR-B, LHR-C and LHR-D.

7. The sCR1 conjugate according to any one of claims 1 to 6, wherein the antigen binding domain binds to or specifically binds to the target and neutralises the signalling.

8. The sCR1 conjugate of claim 7, wherein the target is granulocyte colony stimulating factor (G-CSF) or G-CSF receptor (G-CSFR).

9. The sCR1 conjugate according to any one of claims 1 to 8, wherein the protein binds to or specifically binds to G-CSF or G-CSFR and neutralizes G-CSF signalling.

10. The sCR1 conjugate according to any one of claims 1 to 9, wherein the protein comprises an antigen binding domain of an antibody.

11. The sCR1 conjugate of claim 10, wherein the protein is selected from the group consisting of:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3; or
(x) an antibody.

12. The sCR1 conjugate according to any one of claims 1 to 11, wherein the protein comprises an scFv that binds to or specifically binds to G-CSFR and neutralizes G-CSF signalling.

13. The sCR1 conjugate according to any one of claims 1 to 12, wherein the protein binds to an epitope comprising residues within one or two or three or four regions selected from amino acid residues 111 to 115, 170 to 176, 218 to 234 and/or 286 to 300 of SEQ ID NO: 48.

14. The sCR1 conjugate according to any one of claims 1 to 13, wherein the protein comprises:
(i) a V_{H} comprising an amino acid sequence set forth in SEQ ID NO: 36 and a V_{L} comprising an amino acid sequence set forth in SEQ ID NO: 37;
(ii) a V_{H} comprising an amino acid sequence set forth in SEQ ID NO: 38 and a V_{L} comprising an amino acid sequence set forth in SEQ ID NO: 39; or
(iii) a V_{H} comprising an amino acid sequence set forth in SEQ ID NO: 46 and a V_{L} comprising an amino acid sequence set forth in SEQ ID NO: 47.

15. The sCR1 conjugate of any one of claims 1 to 14, wherein the sCR1 conjugate comprises:
(i) an amino acid sequence corresponding to amino acids 42 to 1649 of SEQ ID NO: 49;
(ii) an amino acid sequence corresponding to amino acids 42 to 1649 of SEQ ID NO: 50;
(iii) an amino acid sequence corresponding to amino acids 42 to 1656 of SEQ ID NO: 51;
(iv) an amino acid sequence corresponding to amino acids 42 to 1656 of SEQ ID NO: 52;
(v) an amino acid sequence corresponding to amino acids 42 to 1648 of SEQ ID NO: 53; or
(vi) an amino acid sequence corresponding to amino acids 42 to 1648 of SEQ ID NO: 54.

16. A soluble complement receptor type 1 (sCR1) conjugate comprising:
(i) an sCR1 variant comprising an amino acid sequence selected from the group consisting of:
a) an amino acid sequence corresponding to amino acids 42 to 939 of SEQ ID NO: 1;
b) an amino acid sequence corresponding to amino acids 490 to 1392 of SEQ ID NO: 1; and
(ii) a protein comprising an antigen binding domain that binds to a blood coagulation factor.

17. The sCR1 conjugate of claim 16, wherein the sCR1 variant comprises:
(i) an amino acid sequence corresponding to amino acids 42 to 1392 of SEQ ID NO: 1;
(ii) an amino acid sequence corresponding to amino acids 42 to 939 of SEQ ID NO: 1;
(iii) an amino acid sequence corresponding to amino acids 490 to 1392 of SEQ ID NO: 1; or
(iv) an amino acid sequence corresponding to amino acids 490 to 1971 of SEQ ID NO: 1.

18. The sCR1 conjugate of claim 16 or 17, wherein the sCR1 variant comprises an amino acid sequence corresponding to amino acids 42 to 1392 of SEQ ID NO: 1.

19. The sCR1 conjugate according to any one of claims 16 to 18, wherein the sCR1 variant has increased complement inhibitory activity compared to a sCR1 comprising a sequence set forth in SEQ ID NO: 2.

20. The sCR1 conjugate according to any one of claims 16 to 19, wherein the sCR1 variant has increased complement inhibitory activity in the classical pathway, the lectin pathway and/or alternative complement pathway compared to a sCR1 comprising a sequence set forth in SEQ ID NO: 2.

21. The sCR1 conjugate according to any one of claims 16 to 20, wherein the sCR1 variant comprises long homologous repeat (LHR) regions selected from the group consisting of:
(i) LHR-A and LHR-B;
(ii) LHR-A, LHR-B and LHR-C;
(iii) LHR-B and LHR-C; and
(iv) LHR-B, LHR-C and LHR-D.

22. The sCR1 conjugate according to any one of claims 16 to 21, wherein the blood coagulation factor is selected from the group consisting of Factor I, Factor II (prothrombin)/thrombin, Factor III, Factor V, Factor VII, Factor VIII, Factor IX, Factor X, Factor XI, Factor XII, Factor XIII and an activated form of any of the foregoing.

23. The sCR1 conjugate according to any one of claims 16 to 22, wherein the antigen binding domain binds to or specifically binds to the blood coagulation factor and antagonises activity and/or antagonises activation of the blood coagulation factor.

24. The sCR1 conjugate according to any one of claims 16 to 23, wherein the blood coagulation factor is Factor XII and/or activated Factor XII (FXIIa).

25. The sCR1 conjugate according to any one of claims 16 to 24, wherein the protein binds to or specifically binds to Factor XII and/or Factor XIIa and antagonises activity of the Factor XII/XIIa and/or antagonises activation of the Factor XII/XIIa.

26. The sCR1 conjugate according to any one of claims 16 to 23, wherein the blood coagulation factor is Factor XI and/or activated Factor XI (FXIa)

27. The sCR1 conjugate according to any one of claim 16 to 23 or 26, wherein the protein binds to or specifically binds to Factor XI and/or Factor XIa and antagonises activity of the Factor XI/XIa and/or antagonises activation of the Factor XII/XIIa.

28. The sCR1 conjugate according to any one of claims 16 to 27, wherein the protein comprises an antigen binding domain of an antibody.

29. The sCR1 conjugate of claim 28, wherein the protein is selected from the group consisting of:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3; or
(x) an antibody.

30. The sCR1 conjugate according to any one of claim 16 to 25, 28 or 29, wherein the protein comprises a scFv that binds to or specifically binds to Factor XII and/or Factor XIIa and antagonises activity of the Factor XII/XIIa and/or antagonises activation of the Factor XII/XIIa.

31. The sCR1 conjugate according to any one of claims 16 to 23 or 26 to 29, wherein the protein comprises a scFv that binds to or specifically binds to Factor XI and/or Factor XIa and antagonises activity of the Factor XI/XIa and/or antagonises activation of the Factor XII/XIIa.

32. The sCR1 conjugate according to any one of claims 16 to 31, wherein the protein comprises:
(i) a V_{H} comprising an amino acid sequence set forth in SEQ ID NO: 56 and a V_{L} comprising an amino acid sequence set forth in SEQ ID NO: 57
(ii) a V_{H} comprising an amino acid sequence set forth in SEQ ID NO: 58 and a V_{L} comprising an amino acid sequence set forth in SEQ ID NO: 59; or
(iii) a V_{H} comprising an amino acid sequence set forth in SEQ ID NO: 60 and a V_{L} comprising an amino acid sequence set forth in SEQ ID NO: 61.

33. The sCR1 conjugate of any one of claims 16 to 32, wherein the sCR1 conjugate comprises:
(i) an amino acid sequence corresponding to amino acids 42 to 1663 of SEQ ID NO: 62;
(ii) an amino acid sequence corresponding to amino acids 42 to 1663 of SEQ ID NO: 63; or
(iii) an amino acid sequence corresponding to amino acids 42 to 1663 of SEQ ID NO: 64.

34. A soluble complement receptor type 1 (sCR1) conjugate comprising:
(i) an sCR1 variant comprising an amino acid sequence selected from the group consisting of:
a) an amino acid sequence corresponding to amino acids 42 to 939 of SEQ ID NO: 1;
b) an amino acid sequence corresponding to amino acids 490 to 1392 of SEQ ID NO: 1; and
(ii) a protein comprising a scFv that binds to or specifically binds to Factor XII and activated Factor XII (FXIIa).

35. A soluble complement receptor type 1 (sCR1) conjugate comprising:
(i) an sCR1 variant comprising an amino acid sequence selected from the group consisting of:
a) an amino acid sequence corresponding to amino acids 42 to 939 of SEQ ID NO: 1;
b) an amino acid sequence corresponding to amino acids 490 to 1392 of SEQ ID NO: 1; and
(ii) a protein comprising a scFv that binds to or specifically binds to Factor XII or activated Factor XII (FXIIa).

36. A soluble complement receptor type 1 (sCR1) conjugate comprising:
(i) an sCR1 variant comprising an amino acid sequence selected from the group consisting of:
a) an amino acid sequence corresponding to amino acids 42 to 939 of SEQ ID NO: 1;
b) an amino acid sequence corresponding to amino acids 490 to 1392 of SEQ ID NO: 1; and
(ii) a protein comprising a scFv that binds to or specifically binds to Factor XI and activated Factor XI (FXIa).

37. A soluble complement receptor type 1 (sCR1) conjugate comprising:
(i) an sCR1 variant comprising an amino acid sequence selected from the group consisting of:
a) an amino acid sequence corresponding to amino acids 42 to 939 of SEQ ID NO: 1;
b) an amino acid sequence corresponding to amino acids 490 to 1392 of SEQ ID NO: 1; and
(ii) a protein comprising a scFv that binds to or specifically binds to Factor XI or activated Factor XI (FXIa).

38. A composition comprising the sCR1 conjugate according to any one of claims 1 to 37, and a pharmaceutical carrier and/or excipient.

39. A sCR1 conjugate according to any one of claims 1 to 15, or a composition 5 according to claim 38, for use in inhibiting complement activity and/or G-CSF activity in a subject.

40. A method of inhibiting complement activity and/or G-CSF activity in a subject, the method comprising administering the sCR1 conjugate according to any one of claims 1 to 15, or the composition according to claim 38

41. Use of the sCR1 conjugate according to any one of claims 1 to 15, or the composition according to claim 38, in the manufacture of a medicament for inhibiting complement activity and/or G-CSF activity in a subject.

42. A sCR1 conjugate according to any one of claims 16 to 35, or a composition according to claim 38, for use in inhibiting complement activity and/or antagonising activity of Factor XII/XIIa and/or antagonising activation of Factor XII/XIIa in a subject.

43. A sCR1 conjugate according to any one of claims 16 to 31 or 36 to 37, or a composition according to claim 38, for use in inhibiting complement activity and/or antagonising activity of Factor XI/XIa and/or antagonising activation of Factor XII/XIIa in a subject.

44. A method of inhibiting complement activity and/or antagonising activity of Factor XII/XIIa and/or antagonising activation of Factor XII/XIIa in a subject, the method comprising administering the sCR1 conjugate according to any one of claims 16 to 35, or the composition according to claim 38.

45. A method of inhibiting complement activity and/or antagonising activity of Factor XII/XIIa and/or antagonising activation of Factor XII/XIIa in a subject, the method comprising administering the sCR1 conjugate according to any one of claims 16 to 31 or 36 to 37, or the composition according to claim 38.

46. Use of the sCR1 conjugate according to any one of claims 1 to 35, or the composition according to claim 38, in the manufacture of a medicament for inhibiting complement activity and/or antagonising activity of Factor XII/XIIa and/or antagonising activation of Factor XII/XIIa in a subject.

47. Use of the sCR1 conjugate according to any one of claims 16 to 31 or 36 to 37, or the composition according to claim 38, in the manufacture of a medicament for inhibiting complement activity and/or antagonising activity of Factor XII/XIIa and/or antagonising activation of Factor XII/XIIa in a subject.

48. A sCR1 conjugate according to any one of claims 1 to 37, or a composition according to claim 38, for use in treating or preventing a disease or condition in a subject.

49. A method of treating or preventing a disease or condition in a subject, the method comprising administering the sCR1 conjugate according to any one of claims 1 to 37, or the composition according to claim 38.

50. Use of the sCR1 conjugate according to any one of claims 1 to 37, or the composition according to claim 38, in the manufacture of a medicament for treating or preventing a disease or condition in a subject.

51. The sCR1 conjugate or composition for use of claim 48, the method of claim of claim 49, or the use of claim 50, wherein the disease or condition is a complement-mediated disorder, a neutrophil-mediated disorder and/or a blood coagulation disorder.

52. The sCR1 conjugate or composition for use according to any one of claim 39, 42, 43, 48 or 51, the method according to any one of claim 40, 44, 45, 49 or 51, or the use according to any one of claim 41, 46, 47, 50 or 51, wherein the subject is suffering from, or at risk of, a complement-mediated disorder, a neutrophil-mediated disorder and/or a blood coagulation disorder.

53. The sCR1 conjugate or composition for use according to any one of claim 39, 42, 43, 48, 51 or 52, the method according to any one of claim 40, 44, 45, 49, 51 or 52, or the use according to any one of claims 41, 46, 47, 50 to 52, wherein the complement-mediated disorder, the neutrophil-mediated disorder and/or the blood coagulation disorder is selected from the group consisting of an inflammatory joint condition, inflammatory arthritis, inflammatory eye condition, inflammatory lung condition, inflammatory neurological condition, autoimmune intestinal disorders, psoriasis, cancer (including angiogenesis thereof) or metastasis thereof, solid organ transplantation (e.g., lung and/or renal transplantation), ischemia reperfusion injury before, during or after transplantation, delayed graft function, asthma and exacerbated forms thereof, neutrophilic dermatosis, a neutrophilic skin lesion, ischemic stroke with reperfusion, neurotraumatic disorder, somatic trauma, ischemia-reperfusion injury (IRI, including myocardial IRI, intestinal IRI, liver IRI and/or pancreatic IRI), venous, arterial or capillary thrombus formation, thrombus formation in the heart, contact-mediated thrombo-inflammation, thrombus formation during and/or after contacting blood of a human or animal subject with artificial surfaces, interstitial lung disease, inflammation, a neurological inflammatory disease, fibrinolysis, angiogenesis, a thrombo-inflammatory disease, a disease related to FXII/FXII-induced kinin formation, atrial fibrillation, acute coronary syndromes (ACS), acute limb ischemia, acute respiratory distress syndrome (ARDS; or acute lung injury) and lupus nephritis (including acute lupus nephritis or chronic lupus nephritis).

54. The sCR1 conjugate or composition for use according to any one of claims 39, 48, 51 to 53, the method according to any one of claims 40, 49, 51 to 53, or the use according to any one of claims 41, 50, 51 to 53, wherein the sCR1 conjugate is administered in an amount sufficient to reduce the number of neutrophils in a subject without inducing neutropenia.

55. A kit for use in inhibiting complement activity and/or G-CSF activity in a subject, the kit comprising:
(a) at least one sCR1 conjugate according to any one of claims 1 to 15, or composition according to claim 38;
(b) instructions for using the kit in inhibiting complement activity and/or G-CSF activity in the subject; and
(c) optionally, at least one further therapeutically active compound or drug.

56. A kit for use in treating or preventing a complement-mediated disorder, a neutrophil-mediated disorder and/or a blood coagulation disorder in a subject, the kit comprising:
(a) at least one sCR1 conjugate according to any one of claims 1 to 15, or composition according to claim 38;
(b) instructions for using the kit in inhibiting complement activity and/or G-CSF activity in the subject; and
(c) optionally, at least one further therapeutically active compound or drug.

57. A kit for use in inhibiting complement activity and/or antagonising activity of Factor XII/XIIa and/or antagonising activation of Factor XII/XIIa in a subject, the kit comprising:
(a) at least one sCR1 conjugate according to any one of claims 16 to 35, or composition according to claim 38;
(b) instructions for using the kit in inhibiting complement activity and/or antagonising activity of Factor XII/XIIa and/or antagonising activation of Factor XII/XIIa in the subject; and
(c) optionally, at least one further therapeutically active compound or drug.

58. A kit for use in inhibiting complement activity and/or antagonising activity of Factor XII/XIIa and/or antagonising activation of Factor XII/XIIa in a subject, the kit comprising:
(a) at least one sCR1 conjugate according to any one of claims 16 to 31 or 36 to 37, or composition according to claim 38;
(b) instructions for using the kit in inhibiting complement activity and/or antagonising activity of Factor XII/XIIa and/or antagonising activation of Factor XII/XIIa in the subject; and
(c) optionally, at least one further therapeutically active compound or drug.

59. A kit for use in treating or preventing a complement mediated disorder, a neutrophil-mediated disorder and/or a blood coagulation disorder in a subject, the kit comprising:
(a) at least one sCR1 conjugate according to any one of claims 16 to 35, or composition according to claim 38;
(b) instructions for using the kit in inhibiting complement activity and/or antagonising activity of Factor XII/XIIa and/or antagonising activation of Factor XII/XIIa in the subject; and
(c) optionally, at least one further therapeutically active compound or drug.

60. A kit for use in treating or preventing a complement mediated disorder, a neutrophil-mediated disorder and/or blood coagulation disorder in a subject, the kit comprising:
(a) at least one sCR1 conjugate according to any one of claims 1 to 31 or 36 to 37, or composition according to claim 38;
(b) instructions for using the kit in inhibiting complement activity and/or antagonising activity of Factor XII/XIIa and/or antagonising activation of Factor XII/XIIa in the subject; and
(c) optionally, at least one further therapeutically active compound or drug.
